(19) United States
(12) Patent Application Publication
(10) Pub. No.: US 2026/0357241 A1
(43) Pub. Date: Nov. 12, 2026

(54) TAGLESS ENZYMATIC THIOETHER MODIFICATIONS

(71) Applicant: University of Utah Research Foundation, Salt Lake City, UT (US)

(72) Inventors: Vahe Bandarian, Salt Lake City, UT (US); Karsten A.S. Eastman, Salt Lake City, UT (US)

(21) Appl. No.: 19/651,342

(22) Filed: Apr. 17, 2026

Related U.S. Application Data
(60) Provisional application No. 63/790,912, filed on Apr. 18, 2025.

(51) Int. Cl. C12N 15/10; C07B 59/00; C07D 283/00; C07D 291/02; C07D 513/08; C07K 5/12; C07K 7/06; C07K 7/64; C07K 14/005

(52) U.S. Cl. CPC C12N 15/1037; C07B 59/008; C07D 283/00; C07D 291/02; C07D 513/08; C07K 5/126; C07K 7/06; C07K 7/64; C07K 14/005; C12N 15/1041; C07B 2200/05; C12N 2795/00022

(57) ABSTRACT

The present disclosure relates to vectors and molecular displays, such as, for example, phage, ribosome, and cell-surface display libraries, and methods of using the disclosed vectors and disclosed molecular displays to provide a peptide sequence with one or more thioether or selenoether linkages. This abstract is intended as a scanning tool for purposes of searching in the particular art and is not intended to be limiting of the present invention.

### CROSS-REFERENCE TO RELATED APPLICATIONS

This Application claims the benefit of U.S. Application No. 63/790,912, filed on Apr. 18, 2025, the contents of which are incorporated herein by reference in their entirety.

### STATEMENT REGARDING FEDERALLY SPONSORED RESEARCH

This invention was made with government support under GM122740 and GM126956 awarded by the National Institutes of Health. The government has certain rights in the invention.

### BACKGROUND

Peptide-based therapeutics are growing due to their unique structure and ability to be produced via solid phase peptide synthesis (SPPS) or by recombinant DNA. Many peptide therapeutics contain a disulfide bond in their active form. Disulfide bonds are susceptible to breakage via biological reductants such as glutathione. Additionally, many peptide therapeutics contain bulky or basic amino acid side chains, which render them vulnerable to degradation by proteases. These factors contribute to their short serum half-lives. Strategies such as L-to-D amino acid swaps, derivatization of the N- and C-termini, N-to-C-terminal cyclization, the introduction of non-proteinogenic amino acids, and metal chelation have both increased peptide half-lives and diversified therapeutic targets. The extent of these modifications is limited to the chemical space afforded by organic synthesis and SPPS.

Nature can access vast chemical space through enzymatic reactions. Natural products are incredibly diverse in their structures, which allow for their wide range of biological and chemical activities. Recent advances in bioinformatic filtering algorithms have uncovered previously unannotated small open reading frames (sORFs). sORFs often colocalize with maturases which further process the peptide after translation. These ribosomally synthesized and post-translationally modified peptides (RiPPs) vary significantly in peptide length, structure, and biological function. RiPP maturases include members of the radical S-adenosylmethionine (rSAM) superfamily. This superfamily has been implicated in a variety of RiPP modifications, including C—C, C—N, C—O and C—S bond formation at unactivated carbons via radical mechanisms. These molecular mechanisms are of substantial interest because they afford access to unique semi-synthetic chemical spaces for production of bioinspired peptide therapeutics. RiPP maturases have potential to offer biotechnological applications in peptide alterations such as thioether installation or peptide stapling. rSAM enzymes use a radical intermediate to complete chemical transformations involved in natural product biosynthesis as well as primary metabolism. These enzymes contain one or more iron-sulfur [Fe-S] clusters that are essential for function. The [4Fe-4S] rSAM (RS) cluster is coordinated by a canonical CxxxCxxC motif in the enzyme. In the [4Fe-4S] RS cluster, one iron coordinates the α-amino and α-carboxylate moieties of SAM. When the RS cluster is catalytically active, it transfers an electron to bound SAM. Either chemical or biological reducing systems are useful for product turnover because the RS cluster is catalytically inactive in the +2 state. Homolytic cleavage of SAM forms the reactive 5′-deoxyadenosyl radical. 5‘-dAdo’ acts as a radical initiator by abstracting a hydrogen atom from a specific site on the substrate, thereby forming 5′-deoxyadenosine and a theoretical RiPP radical intermediate. The formed substrate radical is useful for substrate maturation. While only one [4Fe-4S] cluster is needed for reductive SAM cleavage, many rSAM enzymes also employ one or more auxiliary iron-sulfur clusters (ACs) for substrate turnover (FIG. 4c). These ACs are coordinated to the enzyme by cysteine-rich C-terminal extensions from the RS canonical motif Recent studies have characterized rSAM maturases with multiple [Fe-S] clusters that form intrapeptide bonds between Ca, CB, or Cy on a specific residue and a cysteine thiol in the peptide substrate. Many of these thioether assembling maturases only form a single thioether in the mature peptide and are relatively slow in substrate turnover. The RS cluster in addition to at least one AC cluster is necessary for thioether formation. rSAM RiPP maturases also use a critical RiPP Recognition Element (RRE), that is responsible for binding to the leader sequence of the immature peptide.

PapB is a RiPP maturase that catalyzes the insertion of six thioether crosslinks in the PapA polypeptide. PapB catalyzes the insertion of links between the Cys thiol and the b-carbon of the Asp, where the residues being linked are in a CX₃D motif Prior studies have shown that the enzyme can also accept Glu at the modification site, and that PapB introduces the crosslink to the chemically analogous γ-carbon. In addition, PapB has also been shown to accept a shorter minimal substrate (msPapA), which only has a single pair of crosslinking amino acids in the CX₃D motif PapB can catalyze both CR and Cy thioether linkages, and forms six thioether linkages in the wild type PapA. PapB contains a RS cluster and two ACs. Replacing Asp residue(s) to Glu residue(s) in WT-PapA still results in successful crosslinking. Both CB and Cy thioether linkages were confirmed by 2D NMR.

Despite the emergence of various techniques in peptide-based therapeutics, there remains a need in the art for enzymatic systems for rapid and highly specific modification of a broad range of peptide substances to obtain natural products that are unattainable by traditional synthetic chemistry methods. These needs and others are addressed herein.

### SUMMARY

In accordance with the purpose(s) of the invention, as embodied and broadly described herein, the invention, in one aspect, relates to vectors and molecular displays, such as, for example, phage, ribosome, and cell-surface display libraries, and methods of using the disclosed vectors and disclosed molecular displays to provide a peptide sequence with one or more thioether or selenoether linkages.

Thus, disclosed are vectors comprising a nucleic acid sequence capable of encoding a compound having a structure represented by a formula:

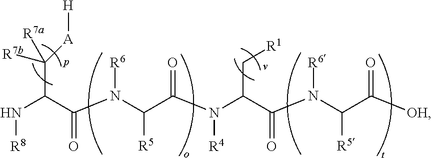

wherein o is 0, 1, 2, 3, 4, 5, 6, 7, 8, or 9; wherein p is 1 or 2; wherein t is an integer from 0 to 500; wherein v is 1, 2, 3, 4, or 5; wherein A is S or Se; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

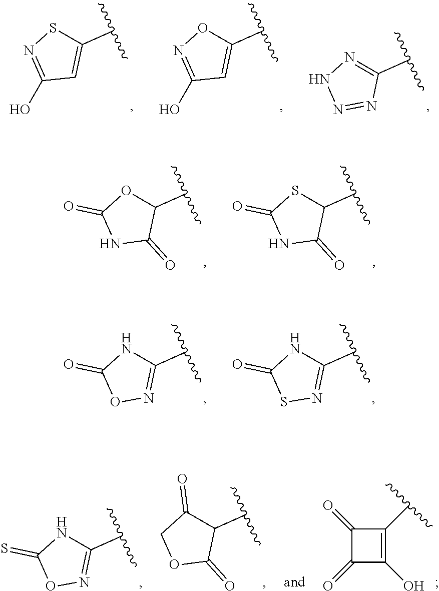

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, provided that the compound is not PapA.

Also disclosed are vectors comprising a nucleic acid sequence capable of encoding a compound having a structure represented by a formula:

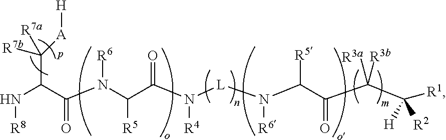

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein A is S or Se; wherein L, when present, is selected from C2-C4 alkyl, —(C1-C4 alkyl)(OCH₂CH₂)q, and a structure selected from:

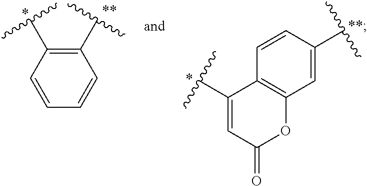

wherein q is 1, 2, 3, or 4; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

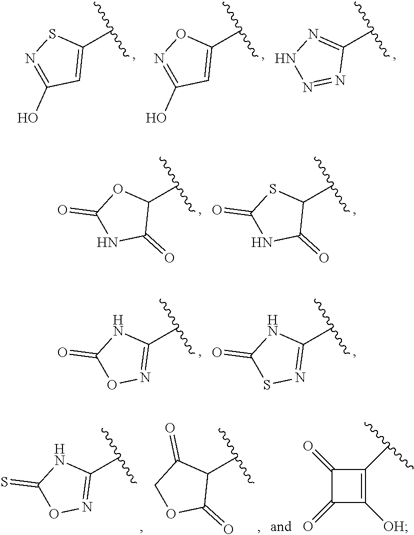

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, provided that the compound is not PapA.

Also disclosed are vectors comprising a nucleic acid sequence capable of encoding a leader sequence and one or more of the compounds disclosed herein.

Also disclosed are phages comprising a compound having a structure represented by a

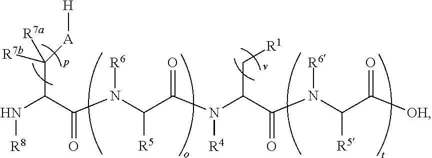

wherein o is 0, 1, 2, 3, 4, 5, 6, 7, 8, or 9; wherein p is 1 or 2; wherein t is an integer from 0 to 500; wherein v is 1, 2, 3, 4, or 5; wherein A is S or Se; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

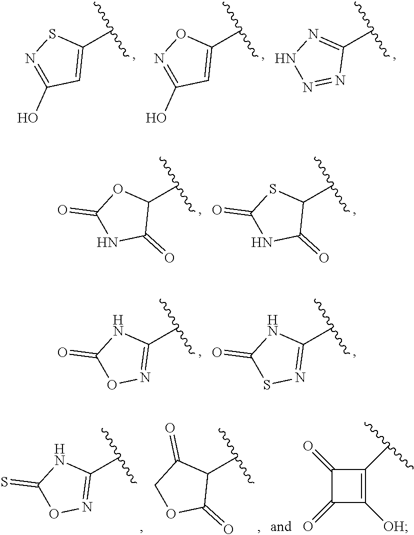

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, provided that the compound is not PapA.

Also disclosed are phages comprising a nucleic acid sequence capable of encoding a compound having a structure represented by a formula:

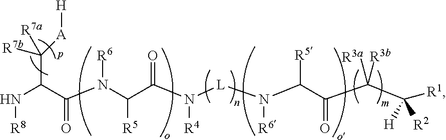

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein A is S or Se; wherein L, when present, is selected from C2-C4 alkyl, —(C1-C4 alkyl)(OCH₂CH₂)_{q}, and a structure selected from:

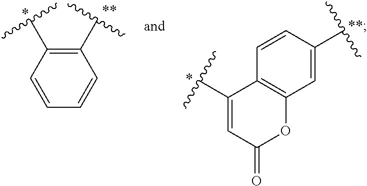

wherein q is 1, 2, 3, or 4; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

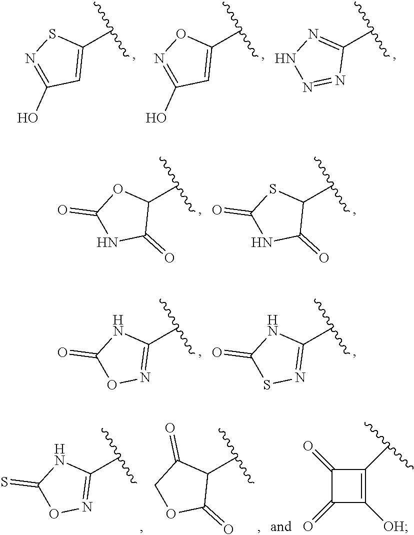

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, provided that the compound is not PapA.

Also disclosed are phages comprising a thioether or selenoether compound, wherein the thioether or selenoether compound has a structure represented by a formula:

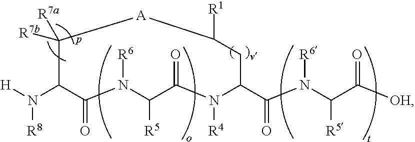

wherein o is 0, 1, 2, 3, 4, 5, 6, 7, 8, or 9; wherein p is 1 or 2; wherein t is an integer from 0 to 500; wherein v′ is 1, 2, 3, or 4; wherein A is S or Se; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

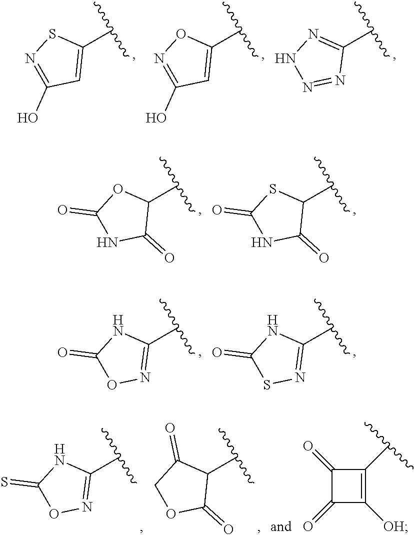

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, provided that the compound is not PapA.

Also disclosed are phages comprising a thioether or seleno ether compound, wherein the thioether or selenoether compound has a structure represented by a formula:

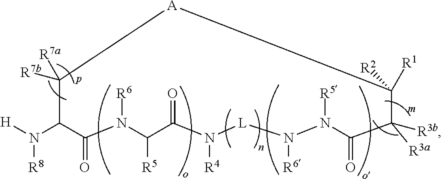

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein A is S or Se; wherein L, when present, is selected from C2-C4 alkyl, —(C1-C4 alkyl)(OCH₂CH₂)_{q}, and a structure selected from:

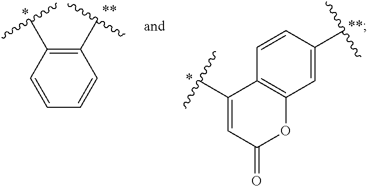

wherein q is 1, 2, 3, or 4; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

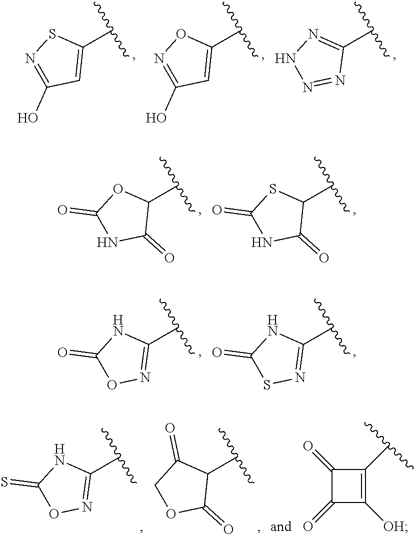

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, provided that the compound is not PapA.

Also disclosed are phage display libraries comprising a compound, wherein each compound has a structure represented by a formula:

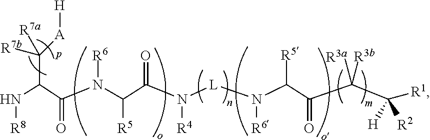

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein A is S or Se; wherein L, when present, is selected from C2-C4 alkyl, —(C1-C4 alkyl)(OCH₂CH₂)_{q}, and a structure selected from:

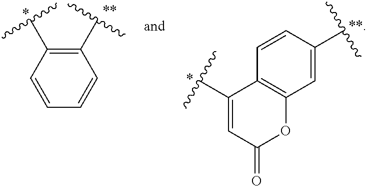

wherein q is 1, 2, 3, or 4; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

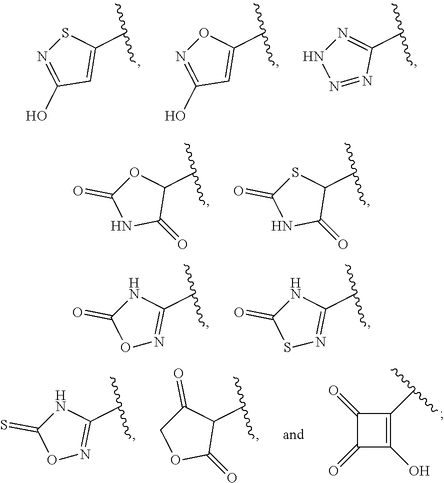

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, provided that the compound is not PapA.

Also disclosed are phage display libraries comprising two or more virus particles, each of the two or more virus particles comprising a thioether or selenoether compound, wherein each thioether or selenoether compound has a structure represented by a formula:

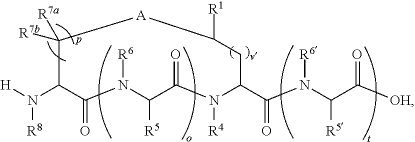

wherein o is 0, 1, 2, 3, 4, 5, 6, 7, 8, or 9; wherein p is 1 or 2; wherein t is an integer from 0 to 500; wherein v′ is 1, 2, 3, or 4; wherein A is S or Se; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

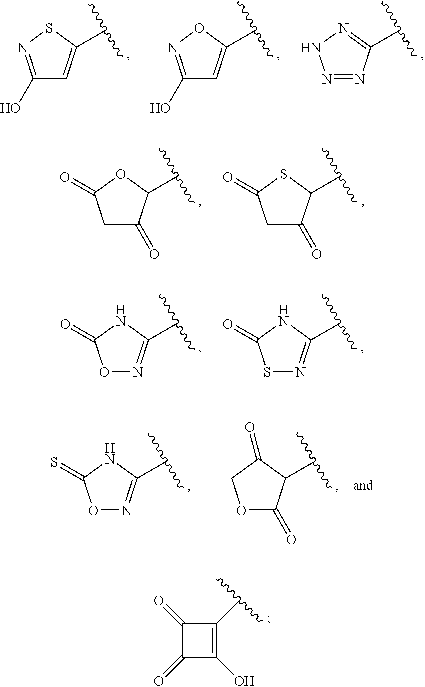

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, provided that the compound is not PapA.

Also disclosed are phage display libraries comprising two or more virus particles, each of the two or more virus particles comprising a compound, wherein each compound has a structure represented by a formula:

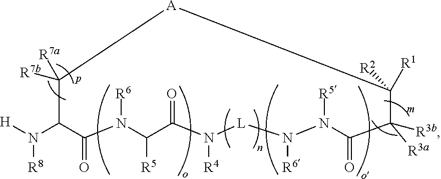

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein A is S or Se; wherein L, when present, is selected from C2-C4 alkyl, —(C1-C4 alkyl)(OCH₂CH₂)_{q}, and a structure selected from:

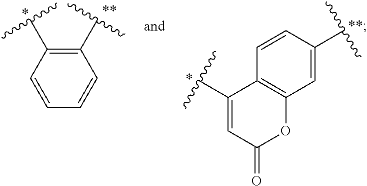

wherein q is 1, 2, 3, or 4; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

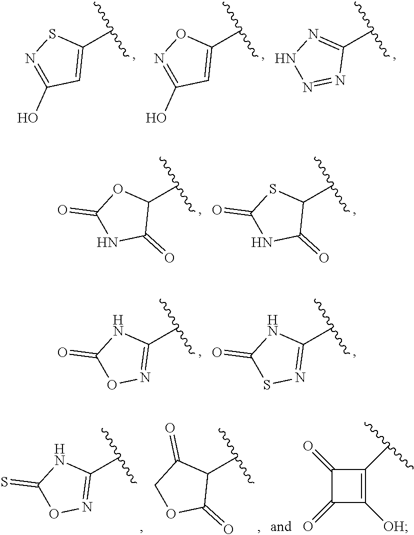

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, provided that the compound is not PapA.

Also disclosed are methods of making a phage display library comprising: (a) introducing one or more of the vectors disclosed herein and a vector comprising a phage genome into a host cell; (b) and culturing the host cells under conditions suitable to form phage particles.

Also disclosed are methods of screening a phage display library, the method comprising: (a) introducing a disclosed vector and a vector comprising a phage genome into a host cell; (b) culturing the host cells under conditions suitable to form phage particles; (c) contacting phage particles with PapB under conditions to allow the compound to form a crosslink, thereby forming a phage particle comprising a crosslinked compound, (d) contacting the phage particles comprising the crosslinked compound with a target molecule to allow binding of the phage particle to the target molecule; (e) separating phage particles that bind to the target molecule from those that do not bind.

Also disclosed are ribosome display libraries comprising a plurality of complexes, wherein each complex comprises: (a) a mRNA molecule, wherein the mRNA molecule comprises a nucleotide sequence capable of encoding one or more of the compounds disclosed herein and lacking an in-frame stop codon; and (b) one or more of the compounds or thioether or selenoether compounds disclosed herein.

Also disclosed are methods of screening using a ribosome display library comprising: (a) obtaining a plurality of mRNA molecules, each mRNA molecule comprising a nucleotide sequence capable of encoding one or more of the compounds disclosed herein and lacking an in-frame stop codon; (b) incubating the plurality of mRNA molecules under conditions for ribosome translation of the mRNA molecules to produce encoded compounds, whereby complexes each comprising the mRNA molecule that encodes a compound, the encoded compound, and a ribosome are formed; (c) contacting the complexes with PapB under conditions to allow the compound to form a crosslink, thereby forming a complex comprising a crosslinked compound (e.g. thioether compound), (d) contacting the complexes with a ligand that binds the compound, (e) selecting one or more complexes each displaying a compound able to bind the ligand; (f) isolating the mRNA molecule from the complex bound to the ligand; and (g) reverse transcribing the mRNA molecule to cDNA.

Also disclosed are cell-surface display libraries comprising a host cell, wherein the host cell comprises one or more of the compounds or thioether or selenoether compounds disclosed herein on the cell surface.

Also disclosed are methods of making a yeast cell surface display, comprising transforming yeast cells with a vector comprising a nucleic acid sequence capable of encoding one or more of the compounds disclosed herein fused to a yeast cell wall protein.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, “IC₅₀” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half-maximal (50%) inhibitory concentration (IC) of a substance.

As used herein, “EC₅₀” is intended to refer to the concentration of a substance (e.g., a compound or a drug) that is required for 50% agonism of a biological process, or component of a process, including a protein, subunit, organelle, ribonucleoprotein, etc. In one aspect, an EC₅₀ can refer to the concentration of a substance that is required for 50% agonism in vivo, as further defined elsewhere herein. In a further aspect, EC₅₀ refers to the concentration of agonist that provokes a response halfway between the baseline and maximum response.

As used herein, the term “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease, disorder, or condition. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the condition being treated and the severity of the condition; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene 9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; anti-cancer and anti-neoplastic agents such as kinase inhibitors, poly ADP ribose polymerase (PARP) inhibitors and other DNA damage response modifiers, epigenetic agents such as bromodomain and extra-terminal (BET) inhibitors, histone deacetylase (HDAc) inhibitors, iron chelotors and other ribonucleotides reductase inhibitors, proteasome inhibitors and Nedd8-activating enzyme (NAE) inhibitors, mammalian target of rapamycin (mTOR) inhibitors, traditional cytotoxic agents such as paclitaxel, dox, irinotecan, and platinum compounds, immune checkpoint blockade agents such as cytotoxic T lymphocyte antigen-4 (CTLA-4) monoclonal antibody (mAB), programmed cell death protein 1 (PD-1)/programmed cell death-ligand 1 (PD-L1) mAB, cluster of differentiation 47 (CD47) mAB, toll-like receptor (TLR) agonists and other immune modifiers, cell therapeutics such as chimeric antigen receptor T-cell (CAR-T)/chimeric antigen receptor natural killer (CAR-NK) cells, and proteins such as interferons (IFNs), interleukins (ILs), and mAbs; anti-ALS agents such as entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “sactipeptide” refers to a sulfur-to-alpha carbon thioether cross-linked peptide belonging to the ribosomally synthesized post-translationally modified peptide (RiPP) superfamily. As illustrated by the structure below, a sactipeptide contains an intramolecular thioether bond that crosslinks the sulfur atom of a cysteine residue to the a-carbon of an acceptor amino acid.

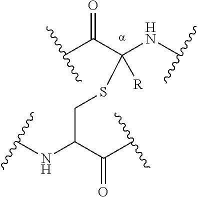

As used herein, the term “ranthipeptide” refers to a radical non-a thioether-containing peptide, which, similar to sactipeptides above, is also a member of the RiPP superfamily. For example, as illustrated below, a ranthipeptide can contain an intramolecular thioether bond that crosslinks the sulfur atom of a cysteine residue to any carbon other than the α-carbon of an acceptor amino acid.

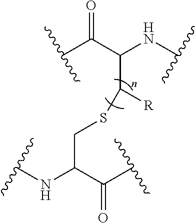

Exemplary ranthipeptide residues containing an β- or γ-carbon are shown below.

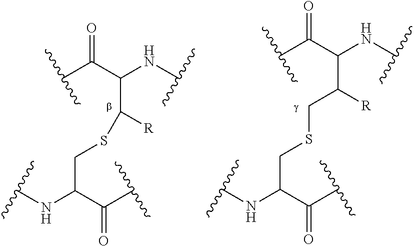

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹ OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond.

Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl)₂ where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide” as used herein can be used interchangeably and refer to F, Cl, Br, or I.

The terms “pseudohalide,” “pseudohalogen,” or “pseudohalo” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein, refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein, refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl” as used herein can be used interchangeably and refer to single and multi-cyclic aromatic or non-aromatic ring systems in which at least one of the ring members is other than carbon. Thus, the term is inclusive of, but not limited to, “heterocycloalkyl”, “heteroaryl”, “bicyclic heterocycle” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxyl” as used herein is represented by the formula OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position.

Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)o-i-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR^{•}₂, =NNHC(O)R^{•}, ═NNHC(O)OR^{•}, ═NNHS(O)₂R*, =NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon-containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

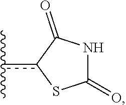

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

The compounds according to this disclosure may form prodrugs at hydroxyl or amino functionalities using alkoxy, amino acids, etc., groups as the prodrug forming moieties. For instance, the hydroxymethyl position may form mono-, di-, or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radio-actively labeled forms, isomers, solvates and combinations thereof. The “combinations” mentioned in this context refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an a-hydrogen can exist in an equilibrium of the keto form and the enol form.

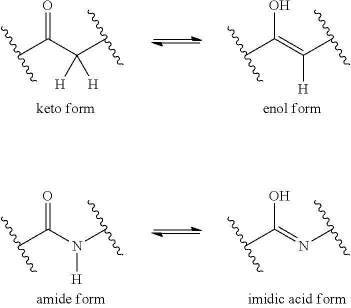 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N¹-unsubstituted, 5-A³ as shown below.

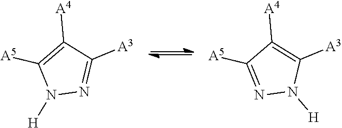

Unless stated to the contrary, the invention includes all such possible tautomers.

It is known that chemical substances form solids, which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

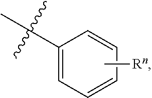

which is understood to be equivalent to a formula:

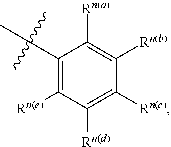

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

The term “coat protein” means a protein, at least a portion of which is present on the surface of the virus particle (e.g. phage). From a functional perspective, a coat protein is any protein which associates with a virus particle during the viral assembly process in a host cell, and remains associated with the assembled virus until it infects another host cell. The coat protein may be the major coat protein or may be a minor coat protein. A “major” coat protein is a coat protein which is present in the viral coat at 10 copies of the protein or more. A major coat protein may be present in tens, hundreds or even thousands of copies per virion.

A “fusion protein” is a polypeptide having two portions covalently linked together, where each of the portions is a polypeptide having a different property. The property may be a biological property, such as activity in vitro or in vivo. The property may also be a simple chemical or physical property, such as binding to a target molecule, catalysis of a reaction, etc. The two portions may be linked directly by a single peptide bond or through a peptide linker containing one or more amino acid residues. Generally, the two portions and the linker will be in reading frame with each other.

“Phage display” is a technique by which polypeptides are displayed on a phage, e.g. filamentous phage, particles. For example, polypeptides can be displayed as fusion proteins to a coat protein on the surface of a phage. A utility of phage display lies in the fact that large libraries of randomized protein variants can be rapidly and efficiently sorted for those sequences that bind to a target molecule with high affinity. Display of peptides and proteins libraries on phage has been used for screening millions of polypeptides for ones with specific binding properties. Polyvalent phage display methods have been used for displaying small random peptides and small proteins through fusions to either gene III or gene VIII of filamentous phage. Wells and Lowman, Curr. Opin. Struct. Biol., 1992, 3:355-362 and references cited therein. In monovalent phage display, a protein or peptide library is fused to a gene III or a portion thereof and expressed at low levels in the presence of wild type gene III protein so that phage particles display one copy or none of the fusion proteins. Avidity effects are reduced relative to polyvalent phage so that sorting is on the basis of intrinsic ligand affinity, and phagemid vectors are used, which simplify DNA manipulations. Lowman and Wells, Methods: A companion to Methods in Enzymology, 1991, 3:205-216.

A “phagemid” is a plasmid vector having a bacterial origin of replication, e.g., ColE1, and a copy of an intergenic region of a bacteriophage. The phagemid may be based on any known bacteriophage, including filamentous bacteriophage and lambdoid bacteriophage. The plasmid will also generally contain a selectable marker for antibiotic resistance. Segments of DNA cloned into these vectors can be propagated as plasmids. When cells harboring these vectors are provided with all genes necessary for the production of phage particles, the mode of replication of the plasmid changes to rolling circle replication to generate copies of one strand of the plasmid DNA and package phage particles. The phagemid may form infectious or non-infectious phage particles. This term includes phagemids which contain a phage coat protein gene or fragment thereof linked to a heterologous polypeptide gene as a gene fusion such that the heterologous polypeptide is displayed on the surface of the phage particle. Sambrook et al., above, 4.17.

The term “phage vector” means a double stranded replicative form of a bacteriophage containing a heterologous gene and capable of replication. The phage vector has a phage origin of replication allowing phage replication and phage particle formation. The phage is preferably a filamentous bacteriophage, such as an M13, f1, fd, Pf3 phage or a derivative thereof, or a lambdoid phage, such as lambda, 21, phi80, phi81, 82, 424, 434, etc., or a derivative thereof.

A “transcription regulatory element” will contain one or more of the following components: an enhancer element, a promoter, an operator sequence, a repressor gene, and a transcription termination sequence. These components are well known in the art. U.S. Pat. No. 5,667,780.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, MA), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compounds and compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. COMPOUNDS

In one aspect, disclosed are compounds having a structure represented by a formula:

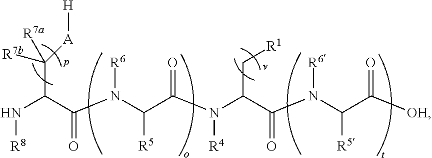

wherein o is 0, 1, 2, 3, 4, 5, 6, 7, 8, or 9; wherein p is 1 or 2; wherein t is an integer from 0 to 500; wherein v is 1, 2, 3, 4, or 5; wherein A is S or Se; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

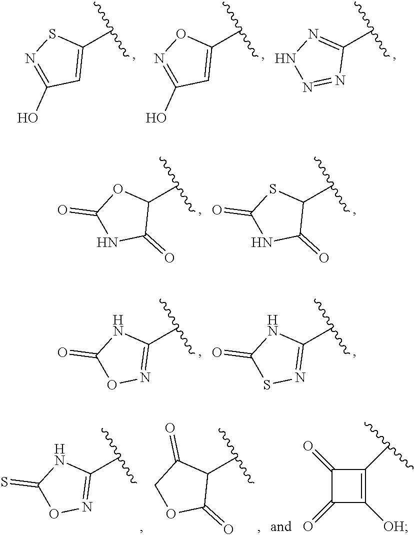

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C₁-C₄ alkyl; and wherein R⁸ is selected from hydrogen and methyl, provided that the compound is not PapA.

In one aspect, disclosed are compounds having a structure represented by a formula:

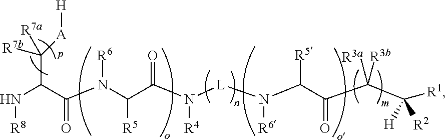

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein A is S or Se; wherein L, when present, is selected from C₂-C₄ alkyl, —(C₁-C₄ alkyl)(OCH₂CH₂)_{q}, and a structure selected from:

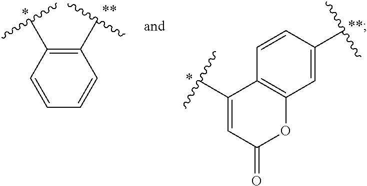

wherein q is 1, 2, 3, or 4; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

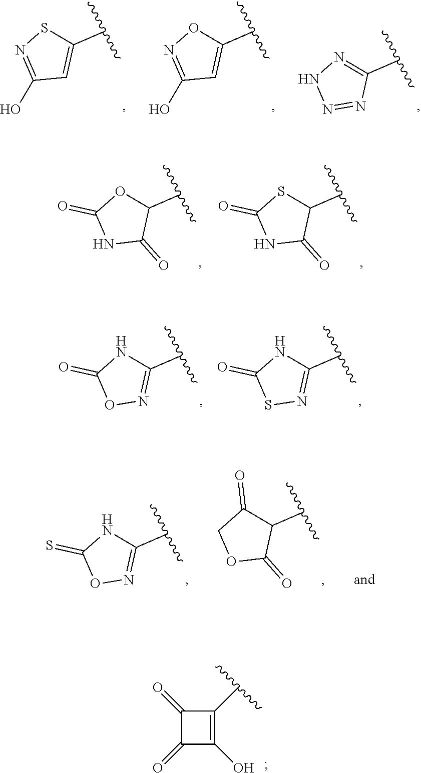

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, provided that the compound is not PapA.

In various aspects, o is independently 0, 1, 2, 3, 4, 5, 6, or 7.

In various aspects, t is 0.

In various aspects, v is 1 or 2.

In various aspects, R¹ is —CO₂H or a structure:

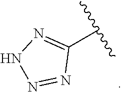 [SMILES: *c1nn[nH]n1]

In various aspects, R¹ is —CO₂H.

In various aspects, the compound comprises one or more D-amino acid residues. In a further aspect, the compound comprises one or more p-amino acid residues. In a still further aspect, the compound comprises one or more N-methylated amino acid residues.

In various aspects, PapB installs a single thioether linkage in the compound.

In a further aspect, PapB installs two or more thioether linkages in the compound.

In various aspects, the compound has a structure represented by a formula:

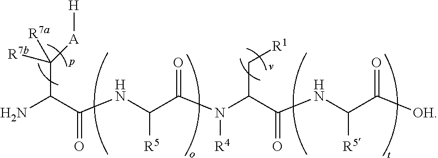

In various aspects, the compound has a structure represented by a formula:

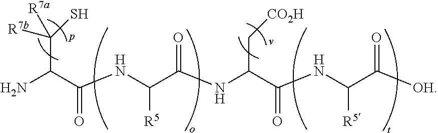

In various aspects, m is 0. In a further aspect, m is 1.

In various aspects, n is 0. In a further aspect, n is 1.

In various aspects, o is 0, 1, 2, 3, 4, 5, 6, or 7. In a further aspect, o is 1, 2, 3, 4, 5, 6, 7, 8, or 9. In a still further aspect, o is 1, 2, 3, or 4.

In various aspects, p is 1. In a further aspect, p is 2.

In various aspects, A is S. In a further aspect, A is Se.

In various aspects, L is C2-C4 alkyl. In a further aspect, L is —(C1-C4 alkyl)(OCH₂CH₂)_{q}. In a still further aspect, L is a structure selected from:

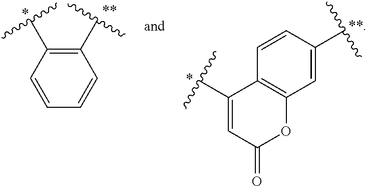 [SMILES: *c1ccccc1* ; *c1ccc2c(*)cc(=O)oc2c1]

In various aspects, R¹ is selected from —CO₂H and a structure:

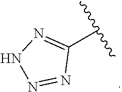 [SMILES: *c1nn[nH]n1]

In various aspects, R¹ is —CO₂H.

In various aspects, R² is a residue of a side chain of an amino acid selected from alanine, valine, leucine, serine, cysteine, methionine, arginine, lysine, asparagine, glycine, phenylalanine, tyrosine, and tryptophan. In a further aspect, R² is a residue of a side chain of an amino acid selected from alanine, leucine, serine, cysteine, methionine, arginine, lysine, asparagine, and glycine.

In various aspects, one of R^{3a} and R^{3b}, when present, is hydrogen, and one of R^{3a} and R^{3b}, when present, is a residue of a side chain of an amino acid selected from alanine, valine, leucine, serine, cysteine, methionine, arginine, lysine, asparagine, glycine, phenylalanine, tyrosine, and tryptophan.

In various aspects, R⁴ is hydrogen. In a further aspect, R⁴ is methyl.

In various aspects, each occurrence of R⁵, when present, is independently a residue of a side chain of an amino acid selected from alanine, valine, leucine, serine, cysteine, methionine, arginine, lysine, asparagine, glycine, phenylalanine, tyrosine, and tryptophan.

In various aspects, each occurrence of R⁶, when present, is hydrogen. In a further aspect, each occurrence of R⁶, when present, is methyl.

In various aspects, each of R^{7a} and R^{7b}, when present, is hydrogen. In a further aspect, each of R^{7a} and R^{7b}, when present, is methyl.

In various aspects, the compound has a structure represented by a formula:

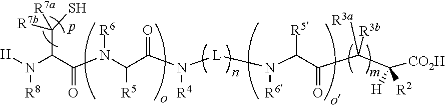

In various aspects, the compound has a structure represented by a formula:

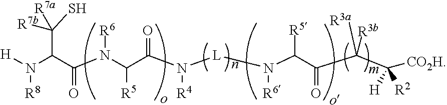

In various aspects, the compound has a structure represented by a formula:

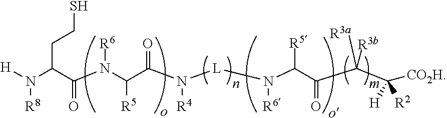

In various aspects, the compound as a structure represented by a formula:

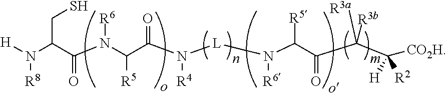

In various aspects, the compound has a structure represented by a formula:

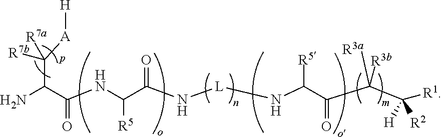

In various aspects, the compound has a structure represented by a formula:

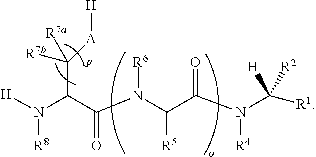

In various aspects, the compound has a structure represented by a formula:

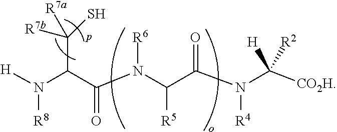

In various aspects, the compound has a structure represented by a formula:

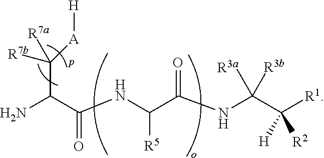

In a further aspect, o is 1, 2, 3, 4, 5, 6, 7, 8, or 9.

In various aspects, one of R^{3a} and R^{3b}, when present, is hydrogen, and one of R^{3a} and R^{3b}, when present, is a residue of a side chain of an amino acid selected from alanine, valine, leucine, serine, cysteine, methionine, arginine, lysine, asparagine, glycine, phenylalanine, tyrosine, and tryptophan.

In various aspects, the compound has a structure represented by a formula:

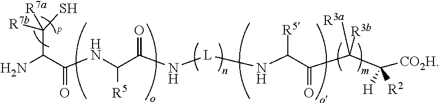

In various aspects, the compound has a structure represented by a formula:

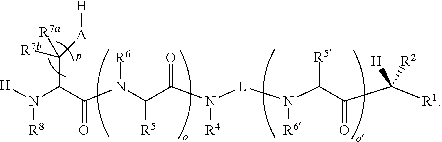

In various aspects, the compound has a structure represented by a formula:

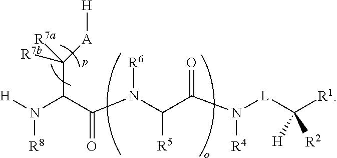

In various aspects, the compound has a structure represented by a formula:

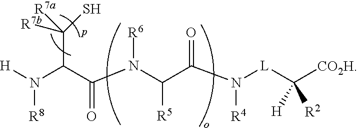

In a further aspect, o is 1, 2, 3, 4, 5, 6, 7, 8, or 9.

In various aspects, the compound has a structure represented by a formula:

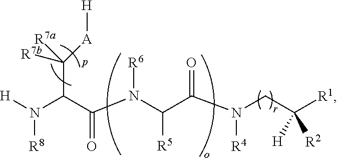

wherein r is 2, 3, or 4.

In various aspects, the compound has a structure represented by a formula:

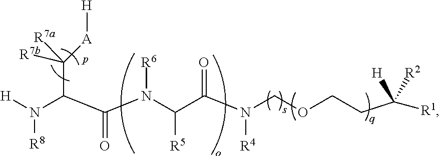

wherein s is 1 or 2.

In various aspects, the compound has a structure represented by a formula:

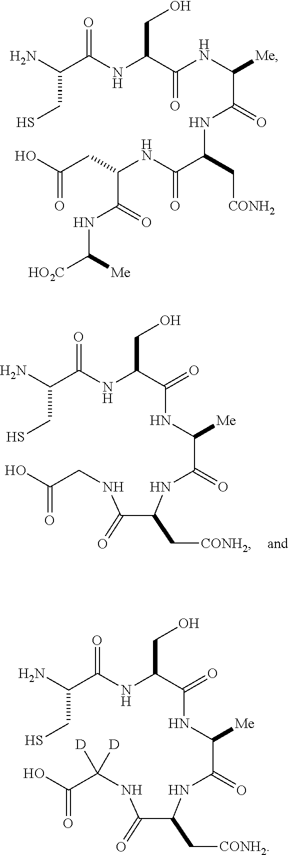 [SMILES: C[C@H](NC(=O)[C@H](CC(=O)O)NC(=O)[C@H](CC(N)=O)NC(=O)[C@H](C)NC(=O)[C@H](CO)NC(=O)[C@@H](N)CS)C(=O)O ; C[C@H](NC(=O)[C@H](CO)NC(=O)[C@@H](N)CS)C(=O)N[C@@H](CC(N)=O)C(=O)NCC(=O)O ; "and" ; [2H]C([2H])(NC(=O)[C@H](CC(N)=O)NC(=O)[C@H](C)NC(=O)[C@H](CO)NC(=O)[C@@H](N)CS)C(=O)O]

### C. THIOETHER AND SELENOETHER COMPOUNDS

In one aspect, disclosed are thioether and selenoether compounds produced by a disclosed method. Thus, in various aspects, the method produces a thioether or a selenoether compound having a structure represented by a formula:

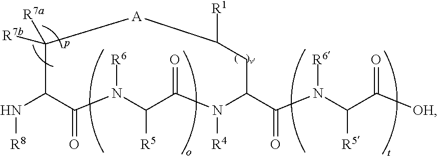

wherein o is 0, 1, 2, 3, 4, 5, 6, 7, 8, or 9; wherein p is 1 or 2; wherein t is an integer from 0 to 500; wherein v′ is 0, 1, 2, 3, or 4; wherein A is S or Se; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

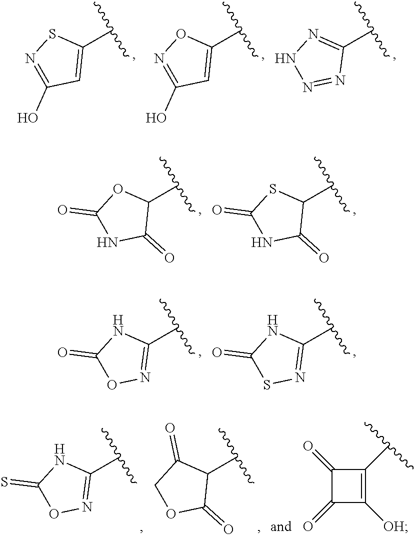

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, provided that the compound is not PapA.

In one aspect, disclosed are thioether and selenoether compounds having a structure represented by a formula:

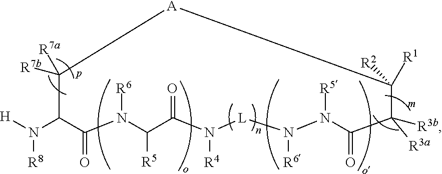

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein A is S or Se; wherein L, when present, is selected from C2-C4 alkyl, —(C1-C4 alkyl)(OCH₂CH₂)_{q}, and a structure selected from:

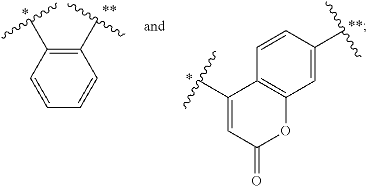

wherein q is 1, 2, 3, or 4; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

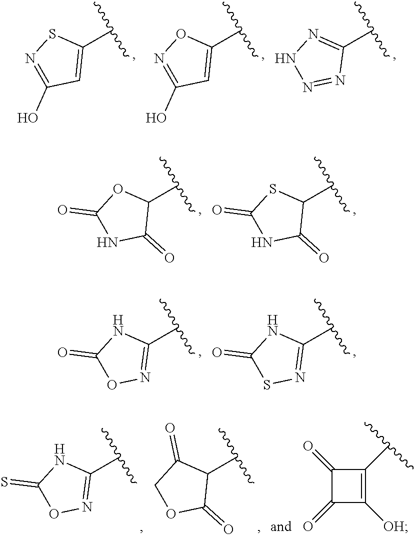

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, provided that the compound is not PapA.

In various aspects, the method further comprises addition of a reducing agent. In a further aspect, the method further comprises addition of a protease.

In various aspects, the method produces a thioether or selenoether compound having a structure represented by a formula:

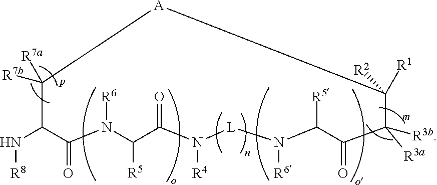

In various aspects, the thioether compound has a structure represented by a formula:

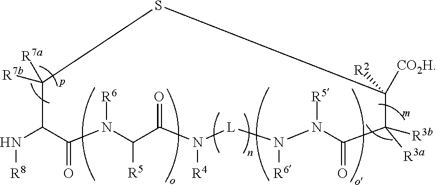

In various aspects, the thioether or selenoether compound has a structure represented by a formula:

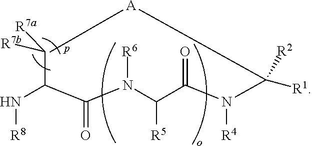

In various aspects, the thioether compound has a structure represented by a formula:

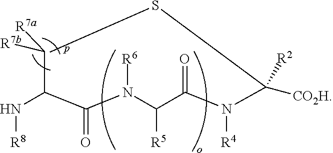

In various aspects, the thioether or selenoether compound has a structure represented by a formula:

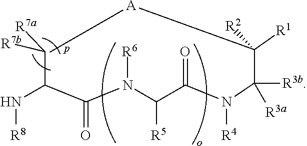

In various aspects, the thioether compound has a structure represented by a formula:

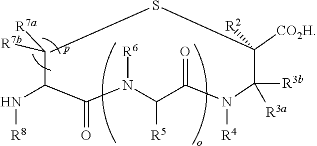

In various aspects, the thioether core selenoether compound has a structure represented by a formula:

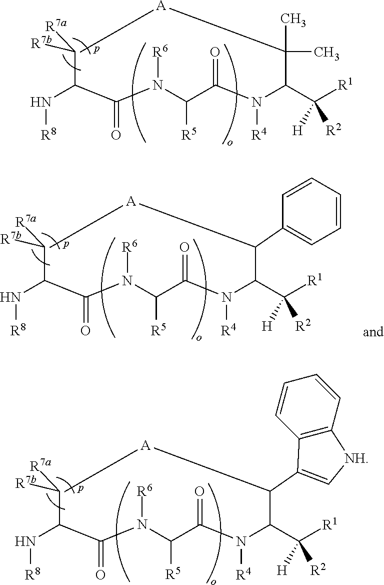

In various aspects, the thioether compound has a structure represented by a formula:

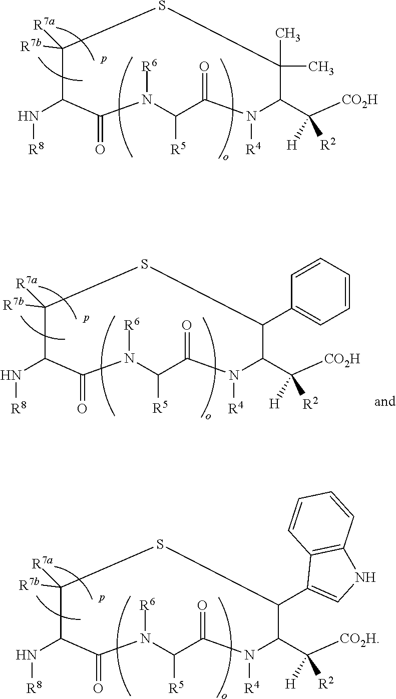

In various aspects, the thioether or selenoether compound has a structure represented by a formula:

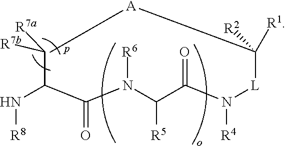

In various aspects, the thioether compound has a structure represented by a formula:

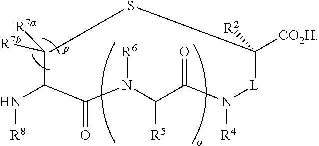

In various aspects, the thioether compound has a structure represented by a formula selected from:

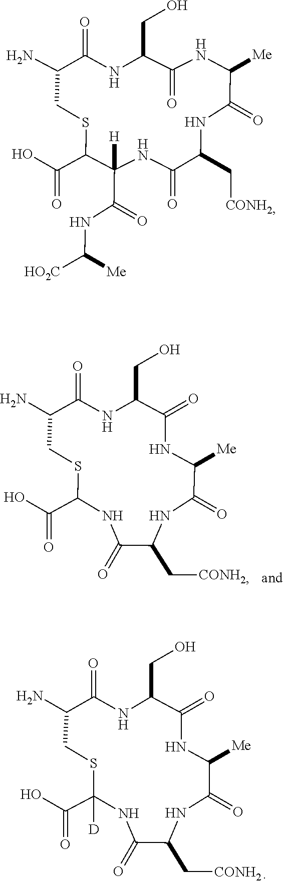 [SMILES: C[C@H](NC(=O)[C@H]1NC(=O)[C@H](CC(N)=O)NC(=O)[C@H](C)NC(=O)[C@H](CO)NC(=O)[C@@H](N)CSC1C(=O)O)C(=O)O ; C[C@@H]1NC(=O)[C@H](CO)NC(=O)[C@@H](N)CSC(C(=O)O)NC(=O)[C@H](CC(N)=O)NC1=O ; [2H]C1(C(=O)O)NC(=O)[C@H](CC(N)=O)NC(=O)[C@H](C)NC(=O)[C@H](CO)NC(=O)[C@@H](N)CS1]

In various aspects, the thioether compound has a structure represented by a formula selected from:

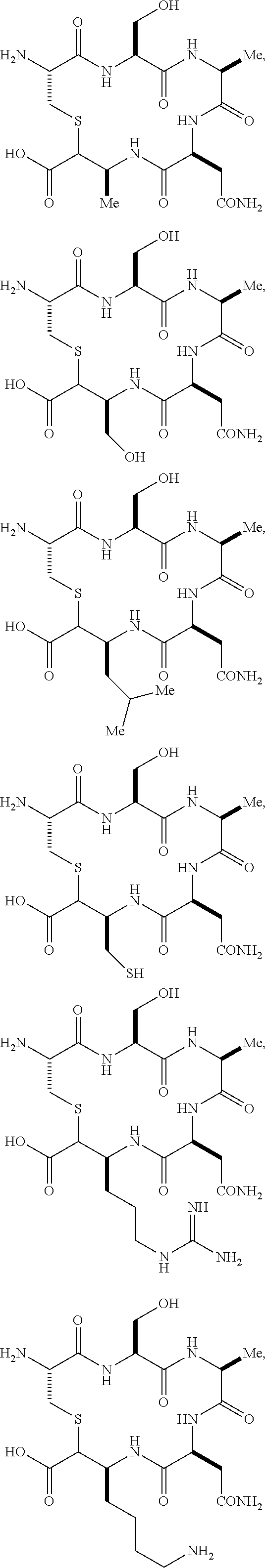

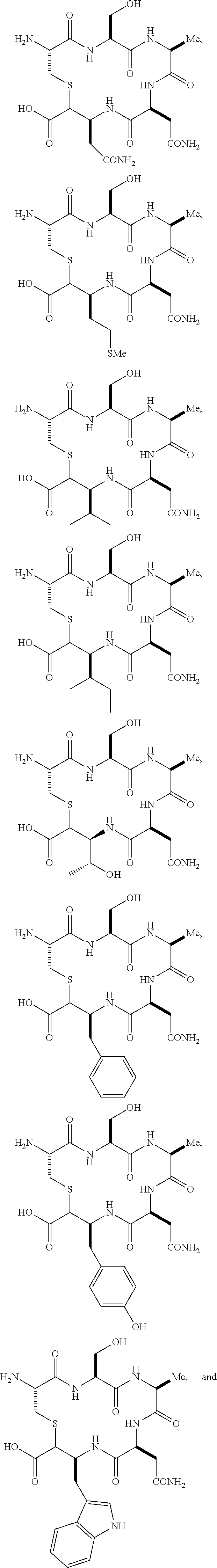

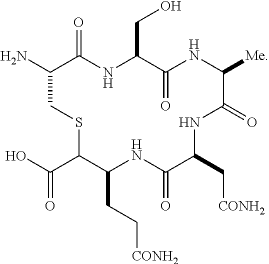 [SMILES: C[C@@H]1NC(=O)[C@H](CO)NC(=O)[C@@H](N)CSC(C(=O)O)[C@H](CCC(N)=O)NC(=O)[C@H](CC(N)=O)NC1=O]

In various aspects, the thioether compound has a structure represented by a formula selected from:

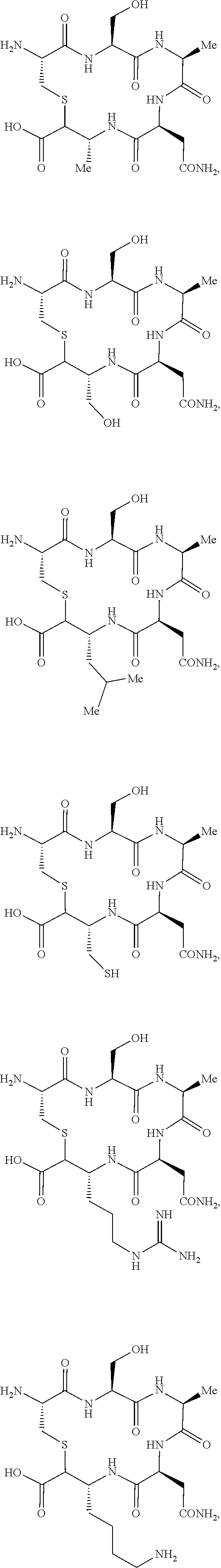

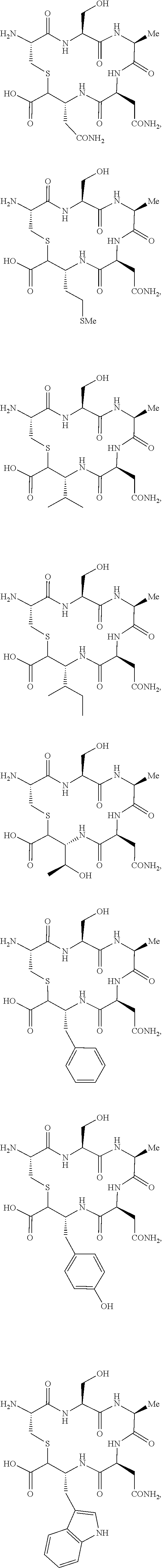

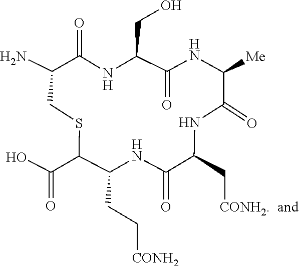

In various aspects, the thioether compound has a structure represented by a formula selected from:

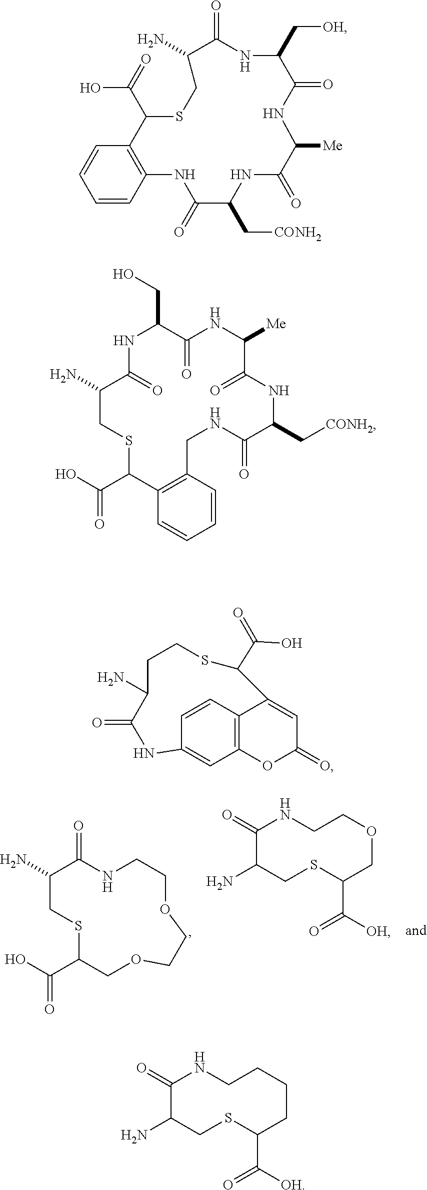 [SMILES: C[C@@H]1NC(=O)[C@H](CO)NC(=O)[C@@H](N)CSC(C(=O)O)c2ccccc2NC(=O)[C@H](CC(N)=O)NC1=O ; C[C@@H]1NC(=O)[C@H](CO)NC(=O)[C@@H](N)CSC(C(=O)O)c2ccccc2CNC(=O)[C@H](CC(N)=O)NC1=O ; NC1CCSC(C(=O)O)c2cc(=O)oc3cc(ccc23)NC1=O ; N[C@H]1CSC(C(=O)O)COCCOCCNC1=O ; NC1CSC(C(=O)O)COCCNC1=O ; NC1CSC(C(=O)O)CCCCNC1=O]

In various aspects, the thioether compound is a sactipeptide. In a further aspect, the sactipeptide has a structure represented by a formula selected from:

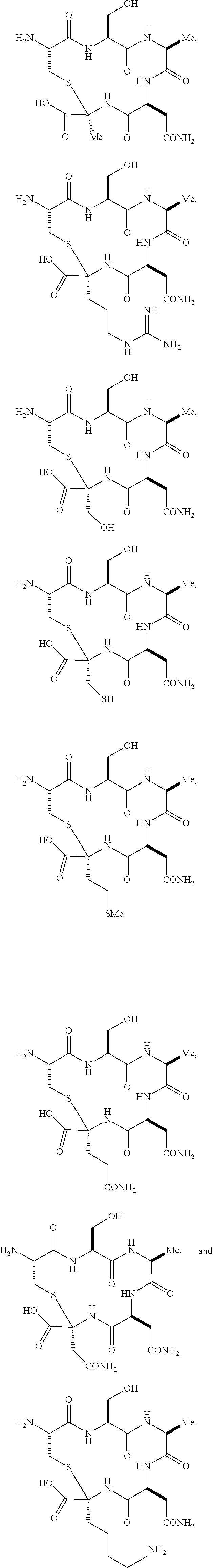

In various aspects, the thioether compound is a ranthipeptide. In a further aspect, the ranthipeptide has a structure represented by a formula selected from:

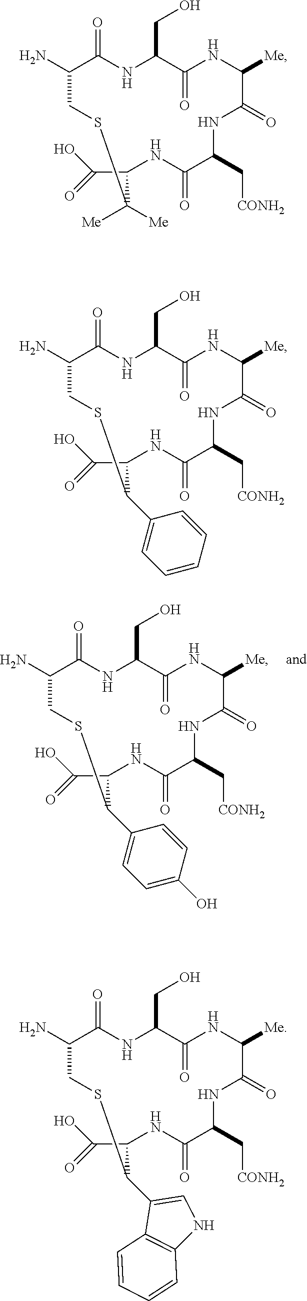

In various aspects, the thioether compound further comprises a molecular display (e.g., a phage displace, a ribosome or mRNA display, or a cell-surface display) covalently attached to either the N-terminus of the C-terminus of the thioether compound as further described herein. Thus, in various aspects, the molecular display can be covalently attached to the N-terminus of the thioether compound. For example, the molecular display can be covalently attached to the leader sequence. Alternatively, in various further aspects, the molecular display can be covalently attached to the C-terminus of the thioether compound. For example, the —(N(R^{6′})CH(R^{5′})C(O))ₜ— group can comprise the molecular display.

### D. VECTORS

Disclosed are vectors comprising a nucleic acid sequence capable of encoding one or more of the disclosed compounds. In some aspects, the term “compound” can be used interchangeably with the term “peptide” since a peptide is a type of compound.

Thus, in one aspect, disclosed are vectors comprising a nucleic acid sequence capable of encoding a compound having a structure represented by a formula:

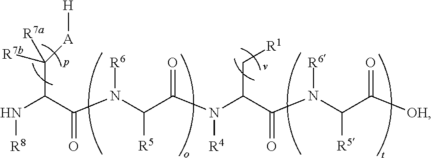

wherein o is 0, 1, 2, 3, 4, 5, 6, 7, 8, or 9; wherein p is 1 or 2; wherein t is an integer from 0 to 500; wherein v is 1, 2, 3, 4, or 5; wherein A is S or Se; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

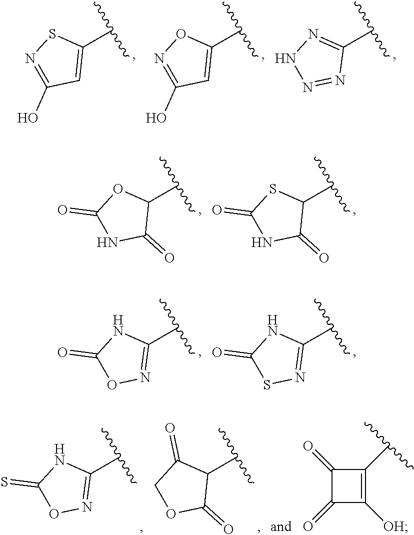

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, provided that the compound is not PapA.

In one aspect, disclosed are vectors comprising a nucleic acid sequence capable of encoding a compound having a structure represented by a formula:

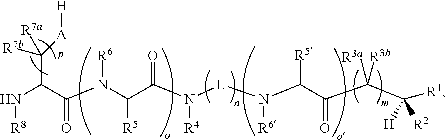

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein A is S or Se; wherein L, when present, is selected from C2-C4 alkyl, —(C1-C4 alkyl)(OCH₂CH₂)_{q}, and a structure selected from:

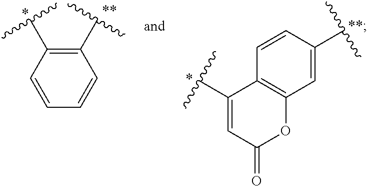

wherein q is 1, 2, 3, or 4; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

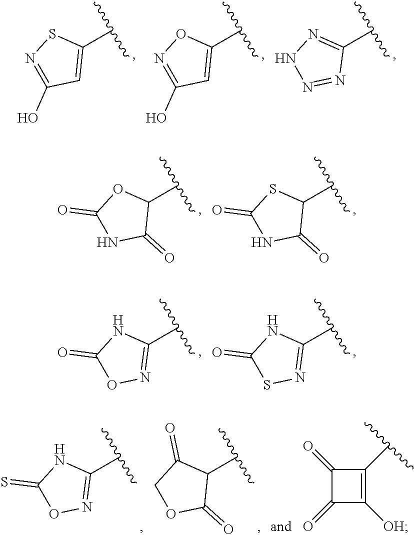

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, provided that the compound is not PapA.

In one aspect, disclosed are vectors comprising a nucleic acid sequence capable of encoding one or more of the compounds disclosed herein.

In various aspects, o is independently 0, 1, 2, 3, 4, 5, 6, or 7.

In various aspects, t is 0.

In various aspects, v is 1 or 2.

In various aspects, w is 0 or 1.

In various aspects, R¹ is —CO₂H or a structure:

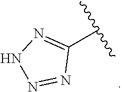 [SMILES: *c1nn[nH]n1]

In various aspects, R¹ is —CO₂H.

In various aspects, the compound comprises one or more D-amino acid residues.

In various aspects, the compound comprises one or more p-amino acid residues.

In various aspects, the compound comprises one or more N-methylated amino acid residues.

In various aspects, the compound has a structure represented by a formula:

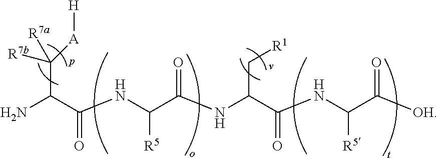

In various aspects, the compound has a structure represented by a formula:

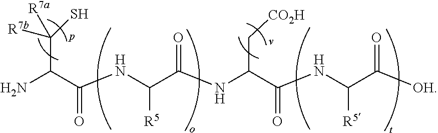

In various aspects, m is 0. In a further aspect, m is 1.

In various aspects, n is 0. In a further aspect, n is 1.

In various aspects, o is 0, 1, 2, 3, 4, 5, 6, or 7. In a further aspect, o is 1, 2, 3, 4, 5, 6, 7, 8, or 9. In a still further aspect, o is 1, 2, 3, or 4.

In various aspects, p is 1. In a further aspect, p is 2.

In various aspects, w is 0. In a further aspect, w is 1.

In various aspects, A is S. In a further aspect, A is Se.

In various aspects, L is C2-C4 alkyl. In a further aspect, L is —(C1-C4 alkyl)(OCH₂CH₂)_{q}. In a still further aspect, L is a structure selected from:

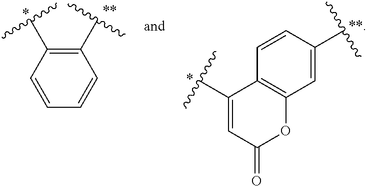 [SMILES: *c1ccccc1* ; *c1ccc2c(*)cc(=O)oc2c1]

In various aspects, R¹ is selected from —CO₂H and a structure:

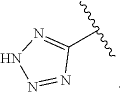 [SMILES: *c1nn[nH]n1]

In various aspects, R¹ is —CO₂H.

In various aspects, R² is a residue of a side chain of an amino acid selected from alanine, valine, leucine, serine, cysteine, methionine, arginine, lysine, asparagine, glycine, phenylalanine, tyrosine, and tryptophan. In a further aspect, R² is a residue of a side chain of an amino acid selected from alanine, leucine, serine, cysteine, methionine, arginine, lysine, asparagine, and glycine.

In various aspects, one of R^{3a} and R^{3b}, when present, is hydrogen, and one of R^{3a} and R^{3b}, when present, is a residue of a side chain of an amino acid selected from alanine, valine, leucine, serine, cysteine, methionine, arginine, lysine, asparagine, glycine, phenylalanine, tyrosine, and tryptophan.

In various aspects, R⁴ is hydrogen. In a further aspect, R⁴ is methyl.

In various aspects, each occurrence of R⁵, when present, is independently a residue of a side chain of an amino acid selected from alanine, valine, leucine, serine, cysteine, methionine, arginine, lysine, asparagine, glycine, phenylalanine, tyrosine, and tryptophan.

In various aspects, each occurrence of R⁶, when present, is hydrogen. In a further aspect, each occurrence of R⁶, when present, is methyl.

In various aspects, each of R^{7a} and R^{7b}, when present, is hydrogen. In a further aspect, each of R^{7a} and R^{7b}, when present, is methyl.

In various aspects, the compound has a structure represented by a formula:

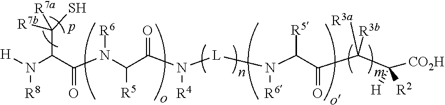

In various aspects, the compound has a structure represented by a formula:

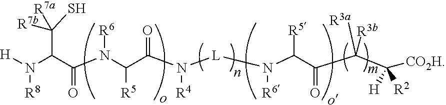

In various aspects, the compound has a structure represented by a formula:

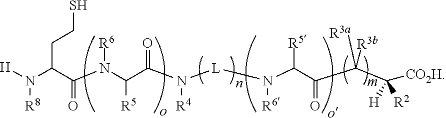

In various aspects, the compound has a structure represented by a formula:

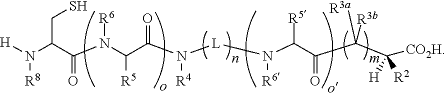

In various aspects, the compound has a structure represented by a formula:

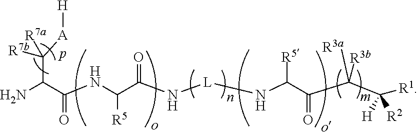

In various aspects, the compound has a structure represented by a formula:

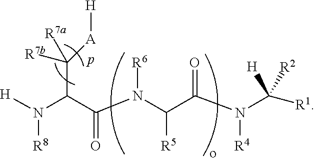

In various aspects, the compound has a structure represented by a formula:

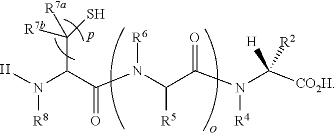

In various aspects, the compound has a structure represented by a formula:

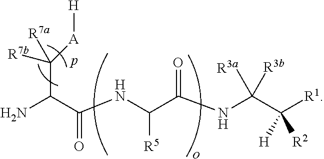

In a further aspect, o is 1, 2, 3, 4, 5, 6, 7, 8, or 9. In a still further aspect, one of R^{3a} and R^{3b} when present, is hydrogen, and one of R^{3a} and R^{3b}, when present, is a residue of a side chain of an amino acid selected from alanine, valine, leucine, serine, cysteine, methionine, arginine, lysine, asparagine, glycine, phenylalanine, tyrosine, and tryptophan.

In various aspects, the compound has a structure represented by a formula:

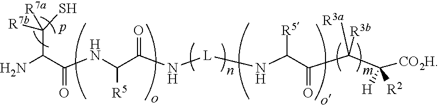

In various aspects, the compound has a structure represented by a formula:

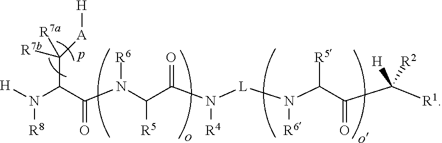

In various aspects, the compound has a structure represented by a formula:

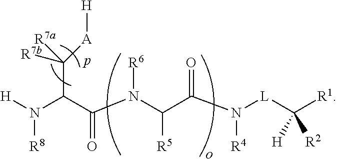

In various aspects, the compound has a structure represented by a formula:

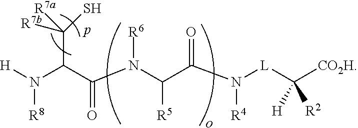

In a further aspect, o is 1, 2, 3, 4, 5, 6, 7, 8, or 9.

In various aspects, the compound has a structure represented by a formula:

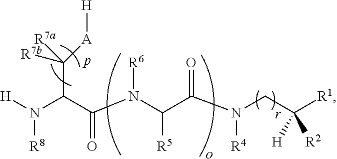

wherein r is 2, 3, or 4.

In various aspects, the compound has a structure represented by a formula:

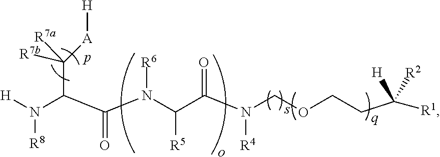

wherein s is 1 or 2.

In various aspects, the compound has a structure represented by a formula:

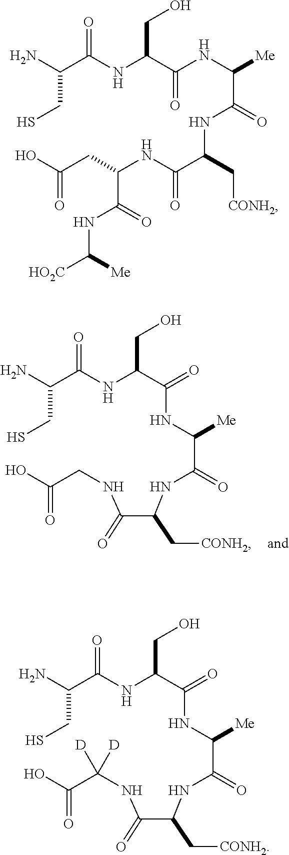 [SMILES: C[C@H](NC(=O)[C@H](CC(=O)O)NC(=O)[C@H](CC(N)=O)NC(=O)[C@H](C)NC(=O)[C@H](CO)NC(=O)[C@@H](N)CS)C(=O)O ; C[C@H](NC(=O)[C@H](CO)NC(=O)[C@@H](N)CS)C(=O)N[C@@H](CC(N)=O)C(=O)NCC(=O)O ; "and" ; [2H]C([2H])(NC(=O)[C@H](CC(N)=O)NC(=O)[C@H](C)NC(=O)[C@H](CO)NC(=O)[C@@H](N)CS)C(=O)O]

In some aspects, any of the disclosed vectors can further comprise a nucleic acid sequence capable of encoding a phage coat protein. Thus, disclosed are vectors comprising a nucleic acid sequence capable of encoding one or more of the compounds disclosed herein and a phage coat protein. In some aspects, these vectors can express a fusion protein comprising one or more of the compounds disclosed herein and a phage coat protein. In some aspects, the nucleic acid sequence is further capable of encoding a linker between the one or more compounds disclosed herein and the phage coat protein. In some aspects, the phage coat protein is a wild type filamentous phage coat protein. In some aspects, the phage coat protein is pIII, pVI, pVII, pVIII, or pIX.

In some aspects, the disclosed vectors further comprise a phage genome. In some aspects, a first vector comprises a nucleic acid sequence capable of encoding one or more of the compounds disclosed herein and a phage coat protein. In some aspects, a second vector comprises a nucleic acid sequence capable of encoding a phage genome. In some aspects, the phage genome in one of the disclosed vectors can be the entire genome minus the phage coat protein that can be provided in trans and capable of encoding a fusion protein with a compound. In some aspects, a single vector comprises a nucleic acid sequence capable of encoding one or more of the compounds disclosed herein, a phage coat protein, and a phage genome.

In some aspects, the vectors comprising a nucleic acid sequence capable of encoding a compound disclosed herein can encode one or more compounds disclosed herein comprising a cysteine residue at the N-terminus of the compound and an aspartate or glutamate residue within twenty amino acids of the cysteine residue. In some aspects, the cysteine residue is N-terminal to an aspartate or glutamate residue within twenty amino acids but does not necessarily have to be at the N-terminus of the compound. For example, if the compound comprises the sequence GGAGAGCASEASAG, the cysteine residue in bold is N-terminal to the glutamic acid but is not on the N-terminal end of the sequence. Furthermore, in some aspects, the one or more compounds disclosed herein can comprise at least two sets of a cysteine residue N-terminal and within twenty amino acids of an aspartate or glutamate residue. Thus, in some aspects the encoded compound can have options for two crosslinks.

For example, if the compound comprises the sequence GGAGACASEASAGCFKFEAAA, the cysteines and the glutamic acids in bold, two crosslinks can be formed; one between the first C to E combination and the other between the second C to E combination.

In some aspects, the nucleic acid sequence capable of encoding one or more of the compounds disclosed herein, wherein the one or more compounds disclosed herein comprise the sequence CXₙD/E, wherein n is between 1 and 20, and wherein X is any amino acid residue. In some aspects, the sequence can further comprise one or more amino acids N-terminal of the cysteine residue. In some aspects, the sequence can further comprise one or more amino acids C-terminal of the D/E.

In some aspects, the nucleic acid sequence capable of encoding one or more of the compounds disclosed herein, wherein the one or more compounds disclosed herein comprise the sequence CXₙX′, wherein n is between 1 and 20, wherein X is any amino acid residue, and wherein X′ is a synthetic (or unnatural) amino acid capable of crosslinking with the C residue. In some aspects, the sequence can further comprise one or more amino acids N-terminal of the cysteine residue. In some aspects, the sequence can further comprise one or more amino acids C-terminal of the X′.

In some aspects, the nucleic acid sequence capable of encoding one or more of the compounds disclosed herein, wherein the one or more compounds disclosed herein comprise a cysteine residue at the N-terminus and a glycine at the C-terminus. In some aspects, any unnatural d-amino acid, or β-amino acid could be at the C-terminus. In some aspects, for crosslinking the Pro-S position must contain an H-atom.

In some aspects, the disclosed vectors can further comprise a transcription regulatory element. In some aspects, the transcription regulatory element can be an enhancer element or a promoter.

In some aspects, the vector is a phagemid or a phage vector.

In some aspects, the vector is an expression vector.

### E. MOLECULAR DISPLAY LIBRARIES

In some aspects, one or more of the disclosed compounds can be displayed in a molecular display library. Examples of molecular display libraries include, but are not limited to, a phage display library, a ribosome display library, and a microbial cell surface display library. Examples of a microbial cell surface display library include, but are not limited to, a yeast cell surface display or bacterial cell surface display.

### 1. Phage Display Libraries

In some aspects, one or more of the disclosed compounds can be displayed on a phage. Thus, in some aspects, one or more of the disclosed compounds can be displayed in a phage display library. In some aspects, the compounds displayed on the phage can be crosslinked using PapB as described herein.

In some aspects, bacteriophage (phage) display is a technique by which variant polypeptides are displayed as fusion proteins to the coat protein on the surface of bacteriophage particles (Scott, J. K. and Smith, G. P. (1990) Science 249: 386). The utility of phage display lies in the fact that large libraries of selectively randomized proteins (or randomly cloned cDNAs) can be rapidly and efficiently sorted for those sequences that bind to a target molecule with high affinity. Display of peptide (Cwirla, S. E. et al. (1990) Proc. Natl. Acad. Sci. USA, 87:6378) or protein (Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363) libraries on phage have been used for screening millions of polypeptides for ones with specific binding properties (Smith, G. P. (1991) Current Opin. Biotechnol., 2:668).

In one aspect, disclosed are phages comprising a compound, wherein the compound has a structure represented by a formula:

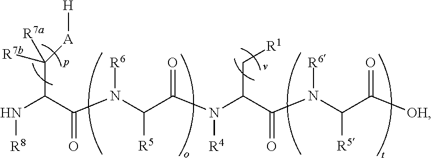

wherein o is 0, 1, 2, 3, 4, 5, 6, 7, 8, or 9; wherein p is 1 or 2; wherein t is an integer from 0 to 500; wherein v is 1, 2, 3, 4, or 5; wherein A is S or Se; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

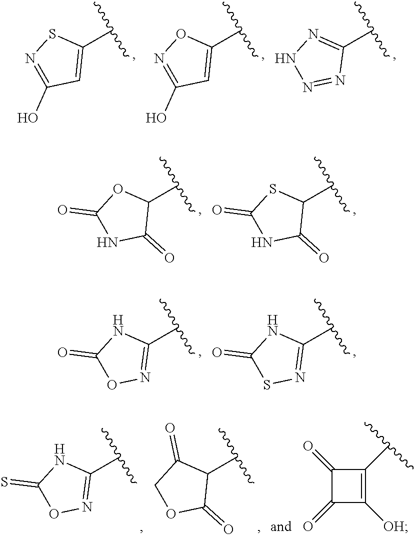

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, provided that the compound is not PapA.

In one aspect, disclosed are phages comprising a compound, wherein the compound has a nucleic acid sequence capable of encoding a compound having a structure represented by a formula:

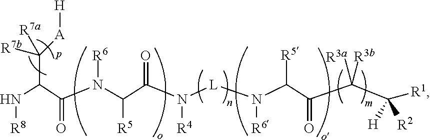

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein A is S or Se; wherein L, when present, is selected from C2-C4 alkyl, —(C1-C4 alkyl)(OCH₂CH₂)_{q}, and a structure selected from:

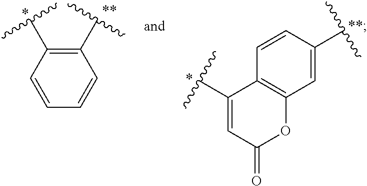

wherein q is 1, 2, 3, or 4; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

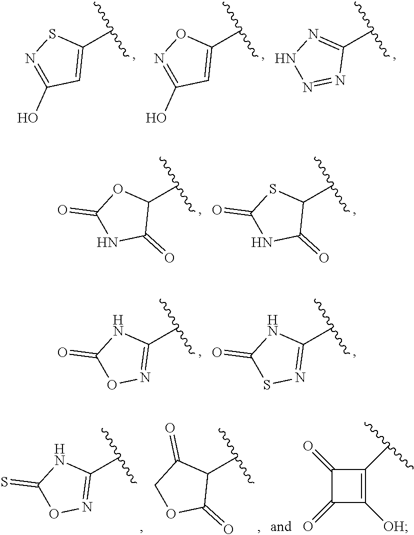

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, provided that the compound is not PapA.

In one aspect, disclosed are phages comprising a thioether or selenoether compound, wherein the thioether or selenoether compound has a structure represented by a formula:

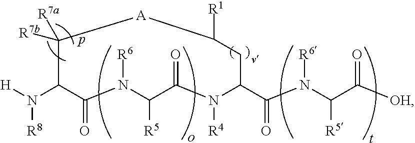

wherein o is 0, 1, 2, 3, 4, 5, 6, 7, 8, or 9; wherein p is 1 or 2; wherein t is an integer from 0 to 500; wherein v′ is 0, 1, 2, 3, or 4; wherein w is 0 or 1; wherein A is S or Se; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

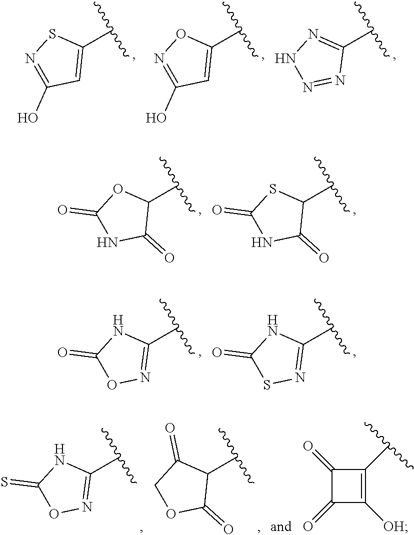

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, provided that the thioether compound is not PapA.

In one aspect, disclosed are phages comprising a thioether or selenoether compound, wherein the thioether or selenoether compound has a structure represented by a formula:

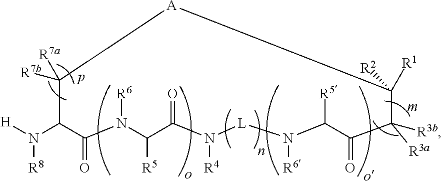

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein w is 0 or 1; wherein A is S or Se; wherein L, when present, is selected from C2-C4 alkyl, —(C1-C4 alkyl)(OCH₂CH₂)_{q}, and a structure selected from:

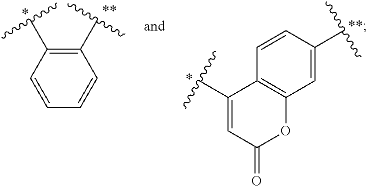

wherein q is 1, 2, 3, or 4; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

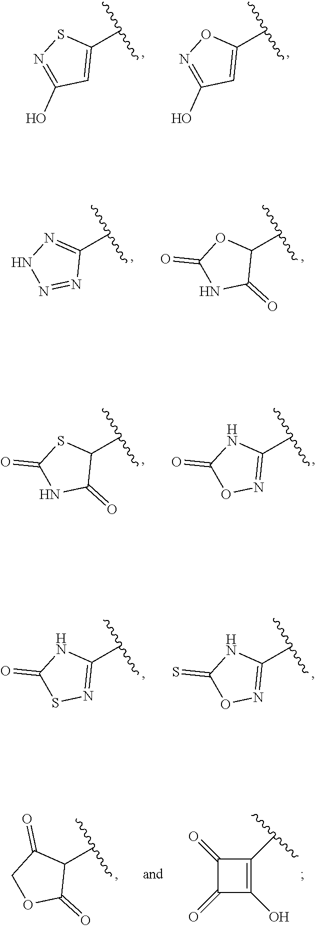

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, provided that the thioether compound is not PapA.

In various aspects, o is independently 0, 1, 2, 3, 4, 5, 6, or 7.

In various aspects, t is 0.

In various aspects, v is 1 or 2.

In various aspects, w is 0 or 1.

In various aspects, R¹ is —CO₂H or a structure:

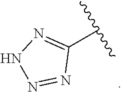 [SMILES: *c1nn[nH]n1]

In various aspects, R¹ is —CO₂H.

In various aspects, the compound comprises one or more D-amino acid residues.

In various aspects, the compound comprises one or more p-amino acid residues.

In various aspects, the compound comprises one or more N-methylated amino acid residues.

In various aspects, the compound has a structure represented by a formula:

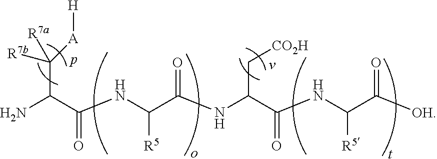

In various aspects, the compound has a structure represented by a formula:

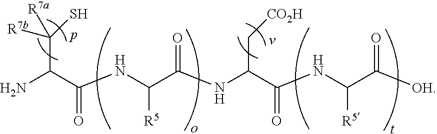

In various aspects, m is 0. In a further aspect, m is 1.

In various aspects, n is 0. In a further aspect, n is 1.

In various aspects, o is 0, 1, 2, 3, 4, 5, 6, or 7. In a further aspect, o is 1, 2, 3, 4, 5, 6, 7, 8, or 9. In a still further aspect, o is 1, 2, 3, or 4.

In various aspects, p is 1. In a further aspect, p is 2.

In various aspects, w is 0. In a further aspect, w is 1.

In various aspects, A is S. In a further aspect, A is Se.

In various aspects, L is C2-C4 alkyl. In a further aspect, L is —(C1-C4 alkyl)(OCH₂CH₂)_{q}. In a still further aspect, L is a structure selected from:

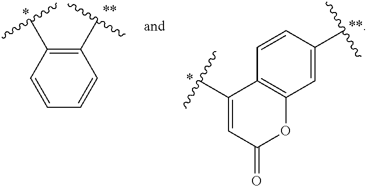 [SMILES: *c1ccccc1* ; *c1ccc2c(*)cc(=O)oc2c1]

In various aspects, R¹ is selected from —CO₂H and a structure:

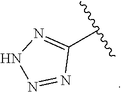 [SMILES: *c1nn[nH]n1]

In various aspects, R¹ is —CO₂H.

In various aspects, R² is a residue of a side chain of an amino acid selected from alanine, valine, leucine, serine, cysteine, methionine, arginine, lysine, asparagine, glycine, phenylalanine, tyrosine, and tryptophan. In a further aspect, R² is a residue of a side chain of an amino acid selected from alanine, leucine, serine, cysteine, methionine, arginine, lysine, asparagine, and glycine.

In various aspects, one of R^{3a} and R^{3b}, when present, is hydrogen, and one of R^{3a} and R^{3b}, when present, is a residue of a side chain of an amino acid selected from alanine, valine, leucine, serine, cysteine, methionine, arginine, lysine, asparagine, glycine, phenylalanine, tyrosine, and tryptophan.

In various aspects, R⁴ is hydrogen. In a further aspect, R⁴ is methyl.

In various aspects, each occurrence of R⁵, when present, is independently a residue of a side chain of an amino acid selected from alanine, valine, leucine, serine, cysteine, methionine, arginine, lysine, asparagine, glycine, phenylalanine, tyrosine, and tryptophan.

In various aspects, each occurrence of R⁶, when present, is hydrogen. In a further aspect, each occurrence of R⁶, when present, is methyl.

In various aspects, each of R^{7a} and R^{7b}, when present, is hydrogen. In a further aspect, each of R^{7a} and R^{7b}, when present, is methyl.

In various aspects, the compound has a structure represented by a formula:

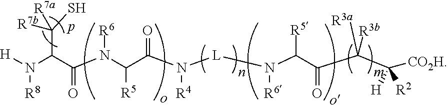

In various aspects, the compound has a structure represented by a formula:

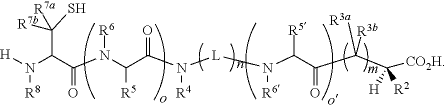

In various aspects, the compound has a structure represented by a formula:

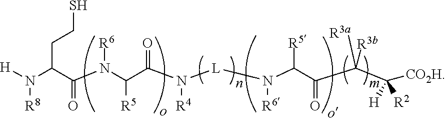

In various aspects, the compound has a structure represented by a formula:

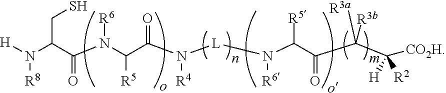

In various aspects, the compound has a structure represented by a formula:

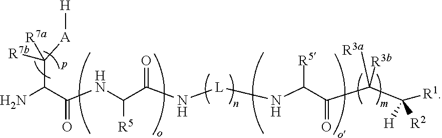

In various aspects, the compound has a structure represented by a formula:

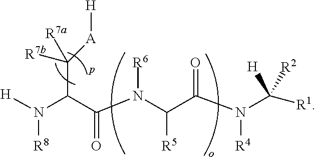

In various aspects, the compound has a structure represented by a formula:

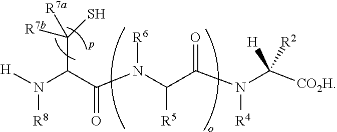

In various aspects, the compound has a structure represented by a formula:

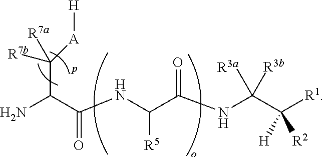

In a further aspect, o is 1, 2, 3, 4, 5, 6, 7, 8, or 9. In a still further aspect, one of R^{3a} and R^{3b} when present, is hydrogen, and one of R^{3a} and R^{3b}, when present, is a residue of a side chain of an amino acid selected from alanine, valine, leucine, serine, cysteine, methionine, arginine, lysine, asparagine, glycine, phenylalanine, tyrosine, and tryptophan.

In various aspects, the compound has a structure represented by a formula:

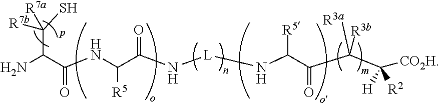

In various aspects, the compound has a structure represented by a formula:

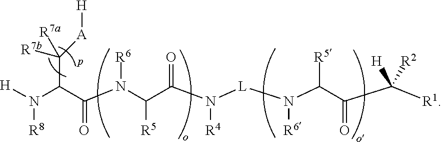

In various aspects, the compound has a structure represented by a formula:

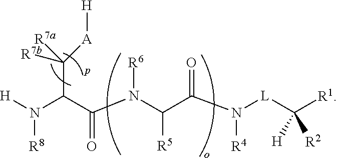

In various aspects, the compound has a structure represented by a formula:

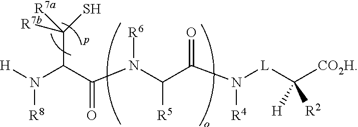

In a further aspect, o is 1, 2, 3, 4, 5, 6, 7, 8, or 9.

In various aspects, the compound has a structure represented by a formula:

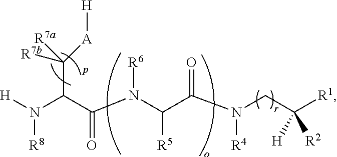

wherein r is 2, 3, or 4.

In various aspects, the compound has a structure represented by a formula:

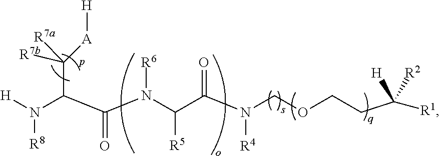

wherein s is 1 or 2.

In various aspects, the compound has a structure represented by a formula:

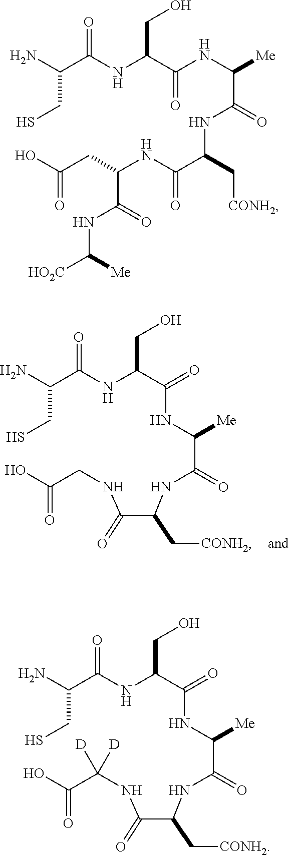 [SMILES: C[C@H](NC(=O)[C@H](CC(=O)O)NC(=O)[C@H](CC(N)=O)NC(=O)[C@H](C)NC(=O)[C@H](CO)NC(=O)[C@@H](N)CS)C(=O)O ; C[C@H](NC(=O)[C@H](CO)NC(=O)[C@@H](N)CS)C(=O)N[C@@H](CC(N)=O)C(=O)NCC(=O)O ; "and" ; [2H]C([2H])(NC(=O)[C@H](CC(N)=O)NC(=O)[C@H](C)NC(=O)[C@H](CO)NC(=O)[C@@H](N)CS)C(=O)O]

In various aspects, the thioether compound has a structure represented by a formula:

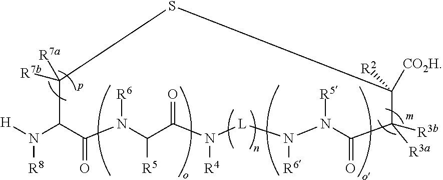

In various aspects, the thioether or selenoether compound has a structure represented by a formula:

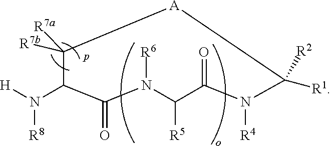

In various aspects, the thioether compound has a structure represented by a formula:

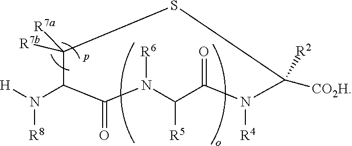

In various aspects, the thioether or selenoether compound has a structure represented by a formula:

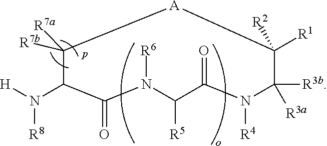

In various aspects, the thioether or selenoether compound has a structure represented by a formula:

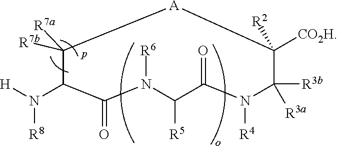

In various aspects, the thioether or selenoether compound has a structure represented by a formula:

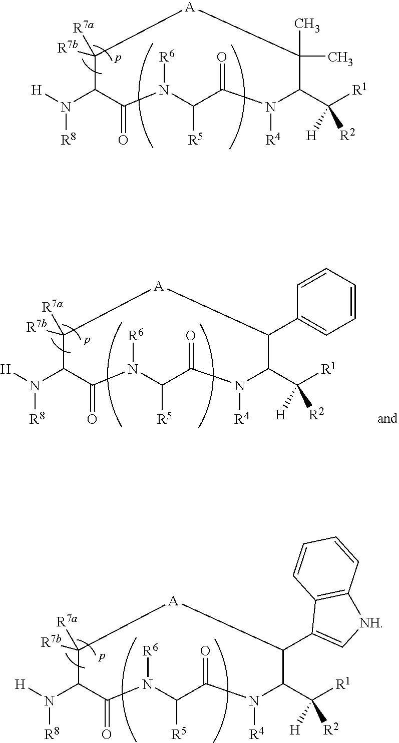

In various aspects, the thioether compound has a structure represented by a formula:

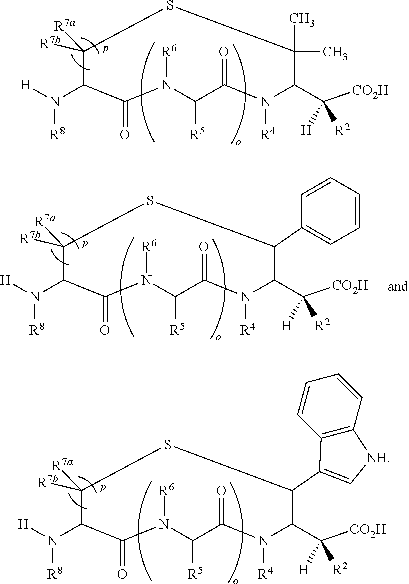

In various aspects, the thioether or selenoether compound has a structure represented by a formula:

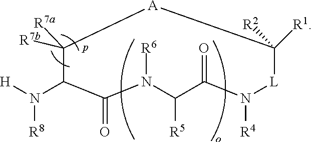

In various aspects, the thioether compound has a structure represented by a formula:

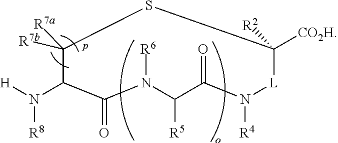

In various aspects, the thioether compound has a structure represented by a formula selected from:

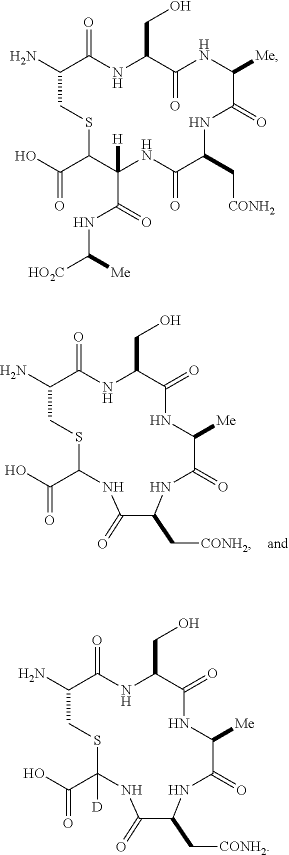 [SMILES: C[C@H](NC(=O)[C@H]1NC(=O)[C@H](CC(N)=O)NC(=O)[C@H](C)NC(=O)[C@H](CO)NC(=O)[C@@H](N)CSC1C(=O)O)C(=O)O ; C[C@@H]1NC(=O)[C@H](CO)NC(=O)[C@@H](N)CSC(C(=O)O)NC(=O)[C@H](CC(N)=O)NC1=O ; "and" ; [2H]C1(C(=O)O)NC(=O)[C@H](CC(N)=O)NC(=O)[C@H](C)NC(=O)[C@H](CO)NC(=O)[C@@H](N)CS1]

In various aspects, the thioether compound has a structure represented by a

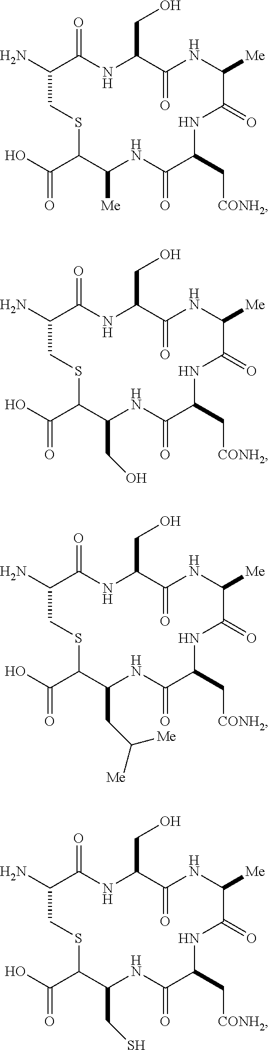 [SMILES: C[C@@H]1NC(=O)[C@H](CO)NC(=O)[C@@H](N)CSC(C(=O)O)[C@H](C)NC(=O)[C@H](CC(N)=O)NC1=O ; C[C@@H]1NC(=O)[C@H](CO)NC(=O)[C@@H](N)CSC(C(=O)O)[C@H](CO)NC(=O)[C@H](CC(N)=O)NC1=O ; CC(C)C[C@@H]1NC(=O)[C@H](CC(N)=O)NC(=O)[C@H](C)NC(=O)[C@H](CO)NC(=O)[C@@H](N)CSC1C(=O)O ; C[C@@H]1NC(=O)[C@H](CO)NC(=O)[C@@H](N)CSC(C(=O)O)[C@H](CS)NC(=O)[C@H](CC(N)=O)NC1=O]

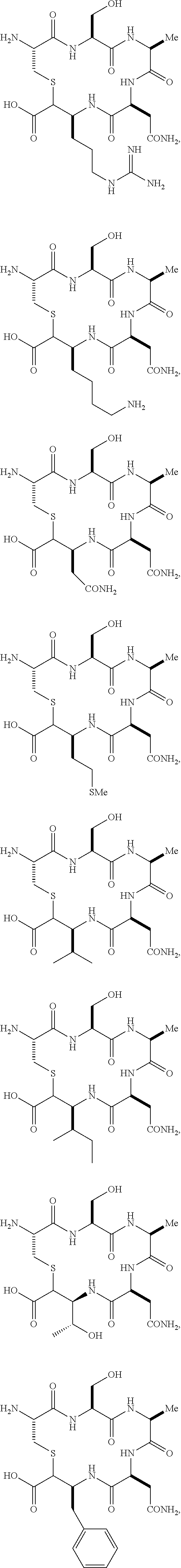

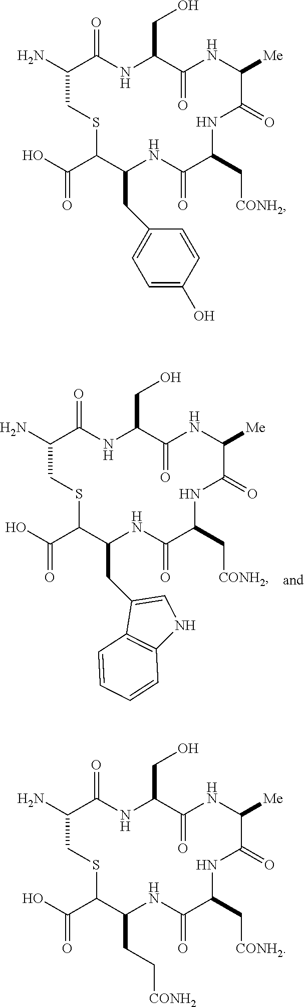 [SMILES: C[C@@H]1NC(=O)[C@H](CO)NC(=O)[C@@H](N)CSC(C(=O)O)[C@H](Cc2ccc(O)cc2)NC(=O)[C@H](CC(N)=O)NC1=O ; C[C@@H]1NC(=O)[C@H](CO)NC(=O)[C@@H](N)CSC(C(=O)O)[C@H](Cc2c[nH]c3ccccc23)NC(=O)[C@H](CC(N)=O)NC1=O ; C[C@@H]1NC(=O)[C@H](CO)NC(=O)[C@@H](N)CSC(C(=O)O)[C@H](CCC(N)=O)NC(=O)[C@H](CC(N)=O)NC1=O]

In various aspects, the thioether compound has a structure represented by a formula selected from:

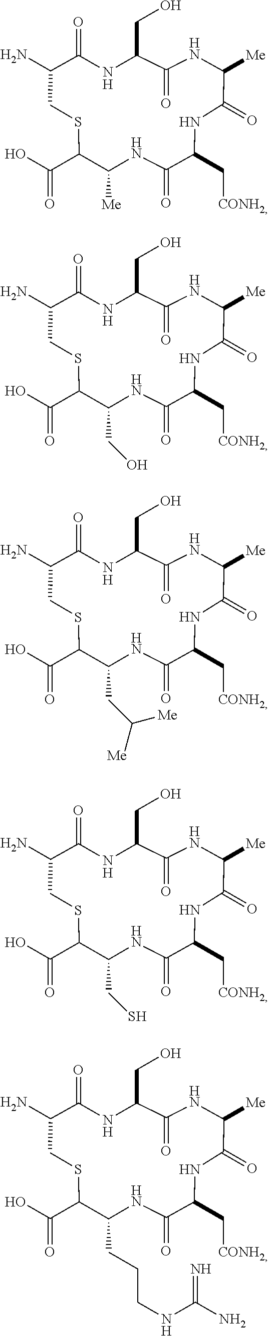

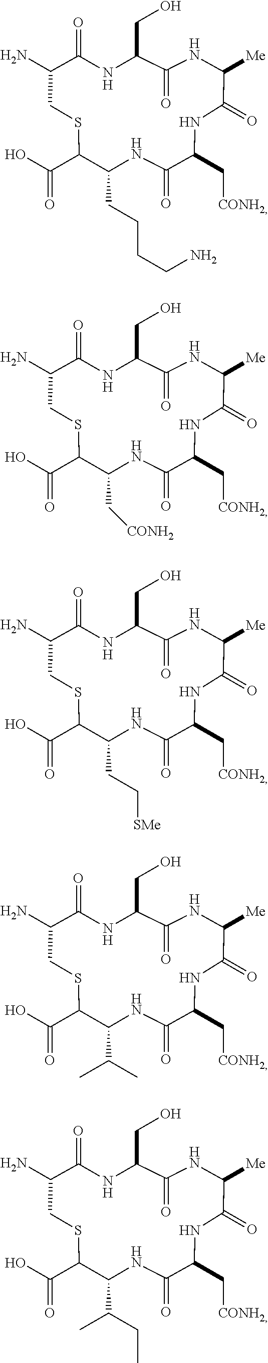

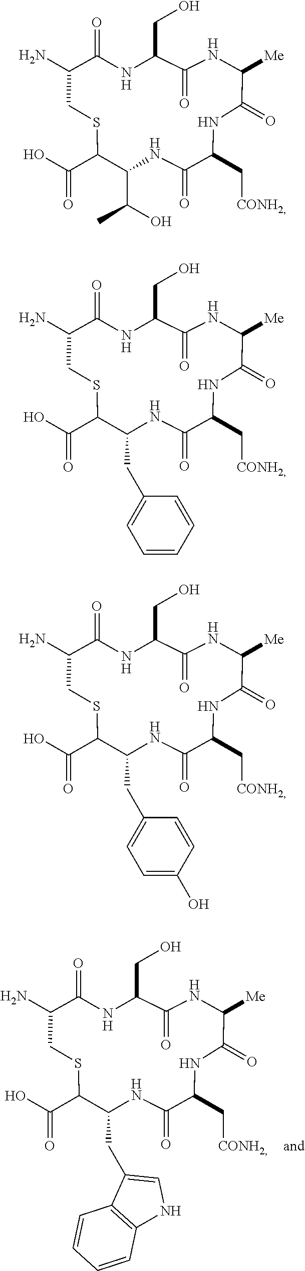

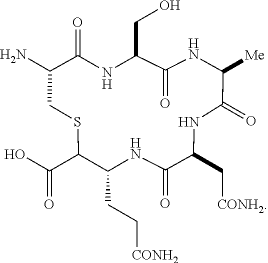 [SMILES: C[C@@H]1NC(=O)[C@H](CO)NC(=O)[C@@H](N)CSC(C(=O)O)[C@@H](CCC(N)=O)NC(=O)[C@H](CC(N)=O)NC1=O]

In various aspects, the thioether compound has a structure represented by a formula selected from:

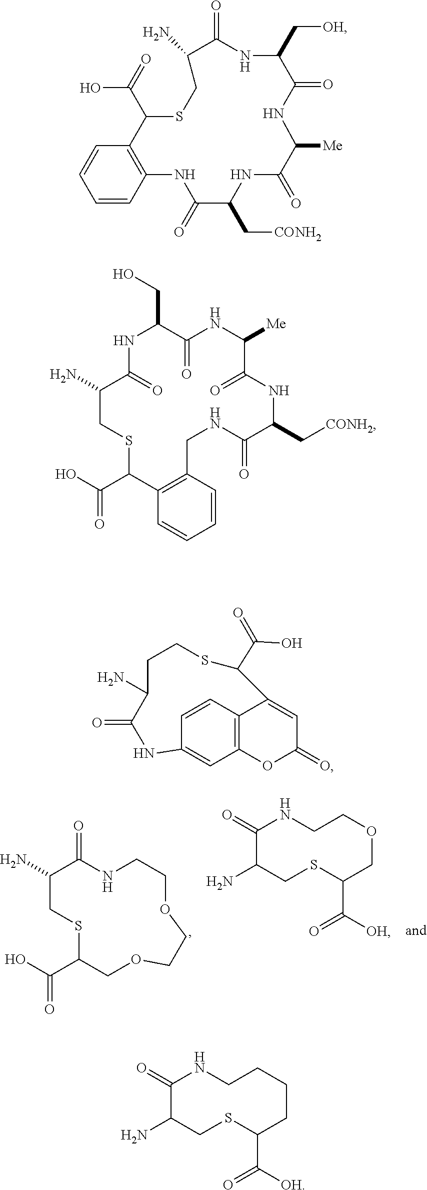 [SMILES: C[C@@H]1NC(=O)[C@H](CO)NC(=O)[C@@H](N)CSC(C(=O)O)c2ccccc2NC(=O)[C@H](CC(N)=O)NC1=O ; C[C@@H]1NC(=O)[C@H](CO)NC(=O)[C@@H](N)CSC(C(=O)O)c2ccccc2CNC(=O)[C@H](CC(N)=O)NC1=O ; NC1CCSC(C(=O)O)c2cc(=O)oc3cc(ccc23)NC1=O ; N[C@H]1CSC(C(=O)O)COCCOCCNC1=O ; NC1CSC(C(=O)O)COCCNC1=O ; NC1CSC(C(=O)O)CCCCNC1=O]

In various aspects, the thioether compound is a sactipeptide. In a further aspect, the sactipeptide has a structure represented by a formula selected from:

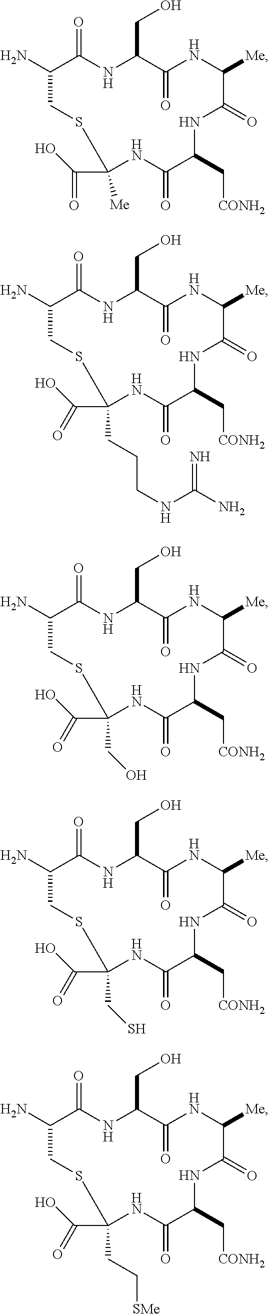

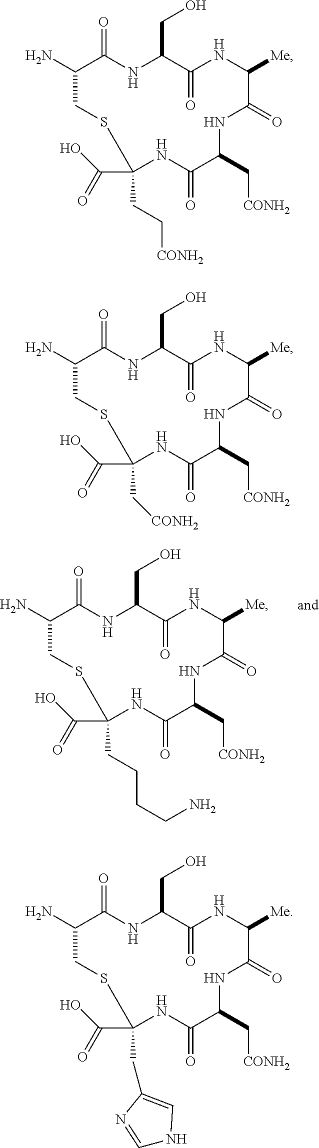

In various aspects, the thioether compound is a ranthipeptide. In a further aspect, the ranthipeptide has a structure represented by a formula selected from:

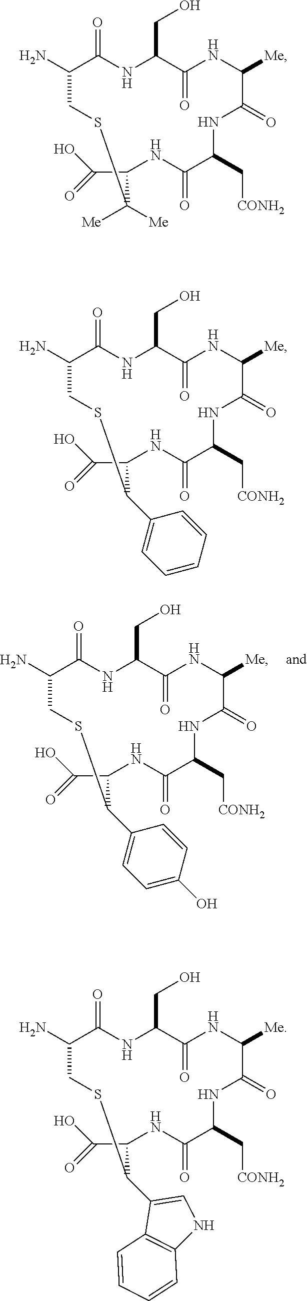

In one aspect, disclosed are phage display libraries comprising two or more of the phage disclosed herein.

In one aspect, disclosed is a phage display library comprising two or more virus particles, each of the two or more virus particles comprising a compound, wherein each compound has a structure represented by a formula:

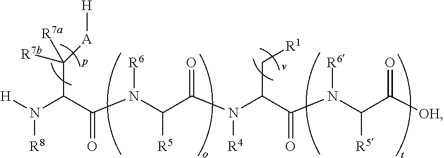

wherein o is 0, 1, 2, 3, 4, 5, 6, 7, 8, or 9; wherein p is 1 or 2; wherein t is an integer from 0 to 500; wherein v is 1, 2, 3, 4, or 5; wherein w is 0 or 1; wherein A is S or Se; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

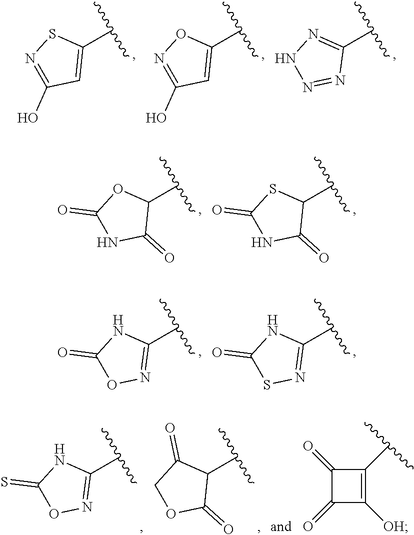

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, provided that the compound is not PapA.

In one aspect, disclosed is a phage display library comprising a compound, wherein the compound has a structure represented by a formula:

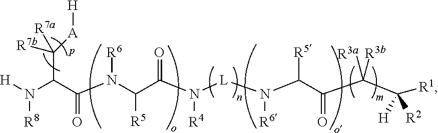

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein w is 0 or 1; wherein A is S or Se; wherein L, when present, is selected from C2-C4 alkyl, —(C1-C4 alkyl)(OCH₂CH₂)_{q}, and a structure selected from:

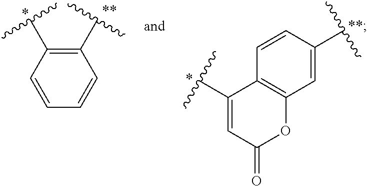

wherein q is 1, 2, 3, or 4; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

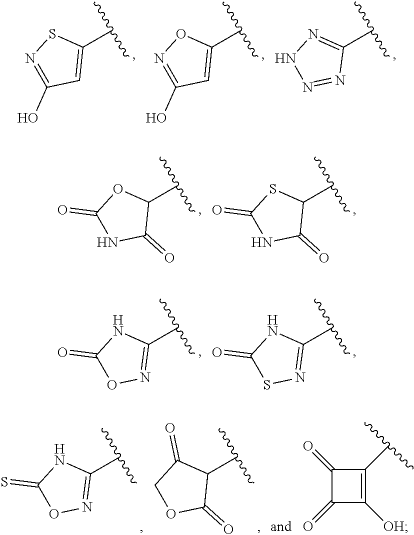

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, provided that the compound is not PapA.

In one aspect, disclosed is a phage display library comprising two or more virus particles, each of the two or more virus particles comprising a thioether or selenoether compound, wherein each thioether or selenoether compound has a structure represented by a formula:

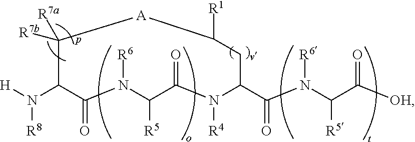

wherein o is 0, 1, 2, 3, 4, 5, 6, 7, 8, or 9; wherein p is 1 or 2; wherein t is an integer from 0 to 500; wherein v′ is 0, 1, 2, 3, or 4; wherein w is 0 or 1; wherein A is S or Se; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

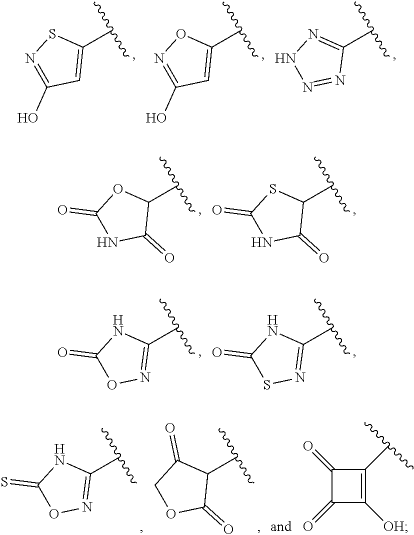

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, provided that the thioether compound is not PapA.

In one aspect, disclosed is a phage display library comprising two or more virus particles, each of the two or more virus particles comprising a thioether or selenoether compound, wherein each thioether or selenoether compound has a structure represented by a formula:

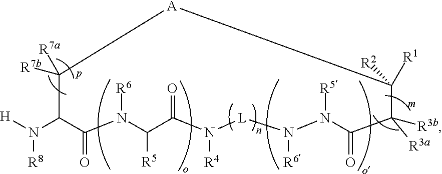

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein w is 0 or 1; wherein A is S or Se; wherein L, when present, is selected from C2-C4 alkyl, —(C1-C4 alkyl)(OCH₂CH₂)_{q}, and a structure selected from:

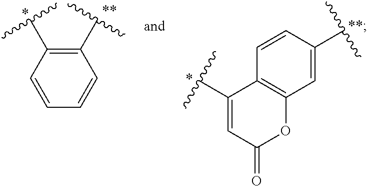

wherein q is 1, 2, 3, or 4; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

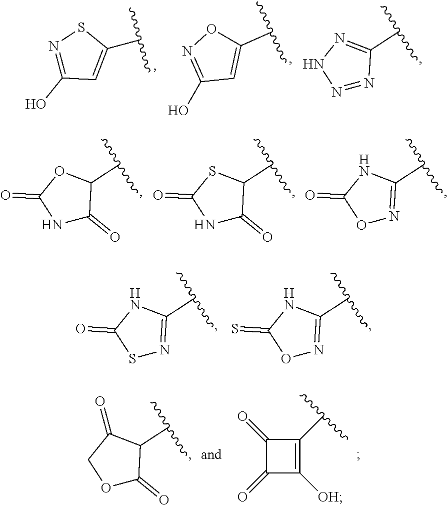

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, provided that the thioether compound is not PapA.

In various aspects, o is independently 0, 1, 2, 3, 4, 5, 6, or 7.

In various aspects, t is 0.

In various aspects, v is 1 or 2.

In various aspects, w is 0 or 1.

In various aspects, R¹ is —CO₂H or a structure:

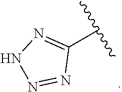 [SMILES: *c1nn[nH]n1]

In various aspects, R¹ is —CO₂H.

In various aspects, the compound comprises one or more D-amino acid residues.

In various aspects, the compound comprises one or more p-amino acid residues.

In various aspects, the compound comprises one or more N-methylated amino acid residues.

In various aspects, the compound has a structure represented by a formula:

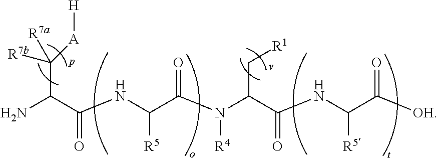

In various aspects, the compound has a structure represented by a formula:

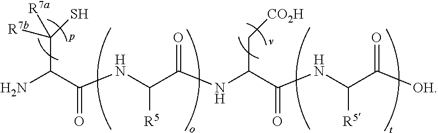

In various aspects, the method produces a thioether or selenoether compound has a structure represented by a formula:

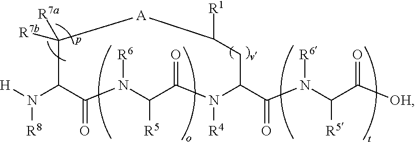

wherein v′ is 0, 1, 2, or 3.

In various aspects, m is 0. In a further aspect, m is 1.

In various aspects, n is 0. In a further aspect, n is 1.

In various aspects, o is 0, 1, 2, 3, 4, 5, 6, or 7. In a further aspect, o is 1, 2, 3, 4, 5, 6, 7, 8, or 9. In a still further aspect, o is 1, 2, 3, or 4.

In various aspects, p is 1. In a further aspect, p is 2.

In various aspects, w is 0. In a further aspect, w is 1.

In various aspects, A is S. In a further aspect, A is Se.

In various aspects, L is C2-C4 alkyl. In a further aspect, L is —(C1-C4 alkyl)(OCH₂CH₂)_{q}. In a still further aspect, L is a structure selected from:

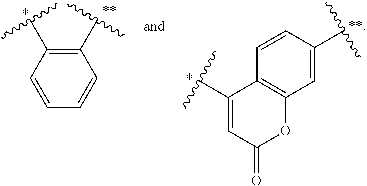 [SMILES: *c1ccccc1* ; *c1ccc2c(*)cc(=O)oc2c1]

In various aspects, R¹ is selected from —CO₂H and a structure:

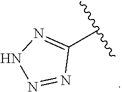 [SMILES: *c1nn[nH]n1]

In various aspects, R¹ is —CO₂H.

In various aspects, R² is a residue of a side chain of an amino acid selected from alanine, valine, leucine, serine, cysteine, methionine, arginine, lysine, asparagine, glycine, phenylalanine, tyrosine, and tryptophan. In a further aspect, R² is a residue of a side chain of an amino acid selected from alanine, leucine, serine, cysteine, methionine, arginine, lysine, asparagine, and glycine.

In various aspects, one of R^{3a} and R^{3b}, when present, is hydrogen, and one of R^{3a} and R^{3b}, when present, is a residue of a side chain of an amino acid selected from alanine, valine, leucine, serine, cysteine, methionine, arginine, lysine, asparagine, glycine, phenylalanine, tyrosine, and tryptophan.

In various aspects, R⁴ is hydrogen. In a further aspect, R⁴ is methyl.

In various aspects, each occurrence of R⁵, when present, is independently a residue of a side chain of an amino acid selected from alanine, valine, leucine, serine, cysteine, methionine, arginine, lysine, asparagine, glycine, phenylalanine, tyrosine, and tryptophan.

In various aspects, each occurrence of R⁶, when present, is hydrogen. In a further aspect, each occurrence of R⁶, when present, is methyl.

In various aspects, each of R^{7a} and R^{7b}, when present, is hydrogen. In a further aspect, each of R^{7a} and R^{7b}, when present, is methyl.

In various aspects, the compound has a structure represented by a formula:

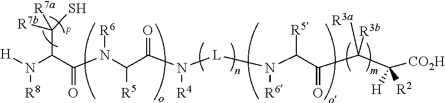

In various aspects, the compound has a structure represented by a formula:

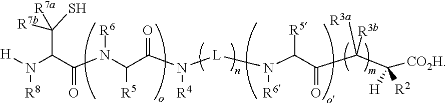

In various aspects, the compound has a structure represented by a formula:

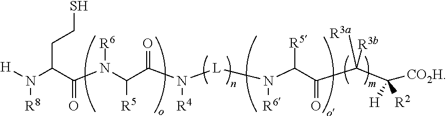

In various aspects, the compound has a structure represented by a formula:

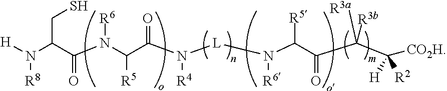

In various aspects, the compound has a structure represented by a formula:

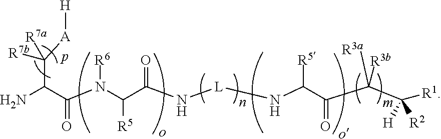

In various aspects, the compound has a structure represented by a formula:

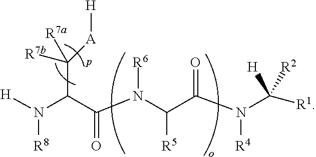

In various aspects, the compound has a structure represented by a formula:

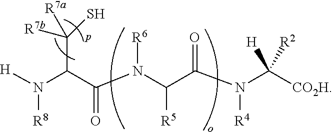

In various aspects, the compound has a structure represented by a formula:

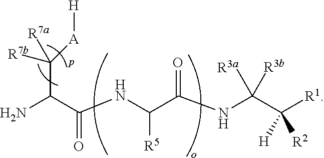

In a further aspect, o is 1, 2, 3, 4, 5, 6, 7, 8, or 9. In a still further aspect, one of R^{3a} and R^{3b} when present, is hydrogen, and one of R^{3a} and R^{3b}, when present, is a residue of a side chain of an amino acid selected from alanine, valine, leucine, serine, cysteine, methionine, arginine, lysine, asparagine, glycine, phenylalanine, tyrosine, and tryptophan.

In various aspects, the compound has a structure represented by a formula:

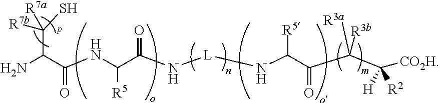

In various aspects, the compound has a structure represented by a formula:

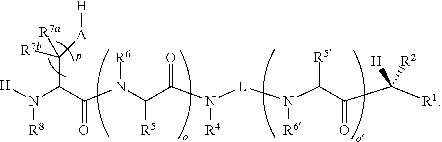

In various aspects, the compound has a structure represented by a formula:

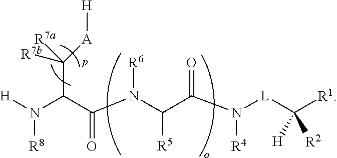

In various aspects, the compound has a structure represented by a formula:

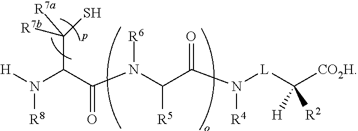

In a further aspect, o is 1, 2, 3, 4, 5, 6, 7, 8, or 9.

In various aspects, the compound has a structure represented by a formula:

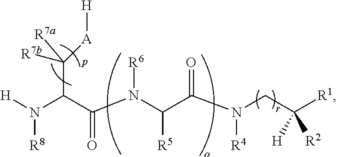

wherein r is 2, 3, or 4.

In various aspects, the compound has a structure represented by a formula:

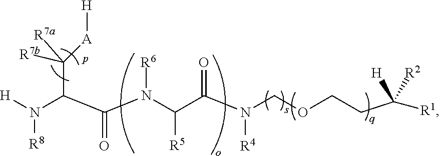

wherein s is 1 or 2.

In various aspects, the compound has a structure represented by a formula:

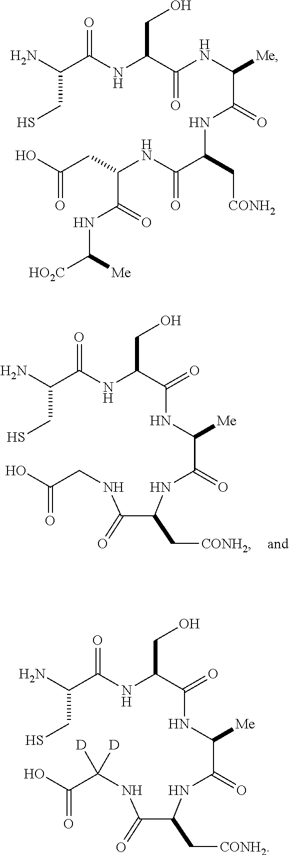 [SMILES: C[C@H](NC(=O)[C@H](CC(=O)O)NC(=O)[C@H](CC(N)=O)NC(=O)[C@H](C)NC(=O)[C@H](CO)NC(=O)[C@@H](N)CS)C(=O)O ; C[C@H](NC(=O)[C@H](CO)NC(=O)[C@@H](N)CS)C(=O)N[C@@H](CC(N)=O)C(=O)NCC(=O)O ; "and" ; [2H]C([2H])(NC(=O)[C@H](CC(N)=O)NC(=O)[C@H](C)NC(=O)[C@H](CO)NC(=O)[C@@H](N)CS)C(=O)O]

In various aspects, the thioether compound has a structure represented by a formula:

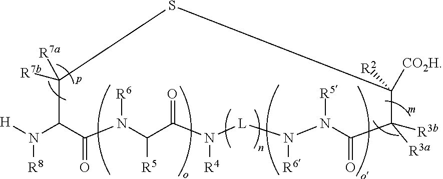

In various aspects, the thioether or selenoether compound has a structure represented by a formula:

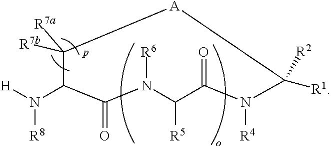

In various aspects, the thioether compound has a structure represented by a formula:

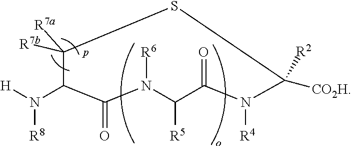

In various aspects, the thioether or selenoether compound has a structure represented by a formula:

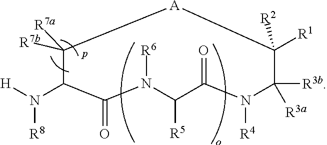

In various aspects, the thioether or selenoether compound has a structure represented by a formula:

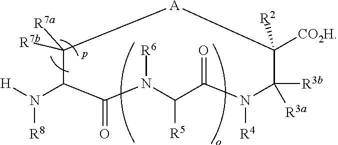

In various aspects, the thioether or selenoether compound has a structure represented by a formula:

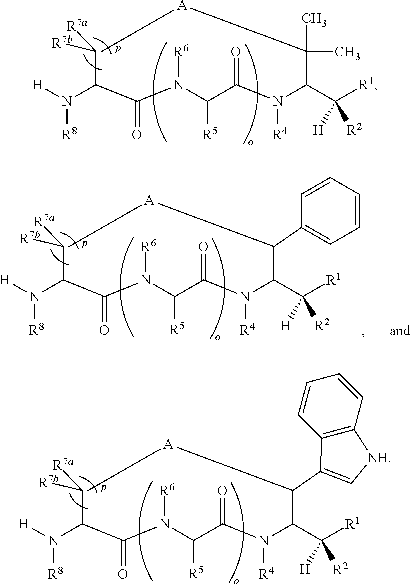

In various aspects, the thioether compound has a structure represented by a formula:

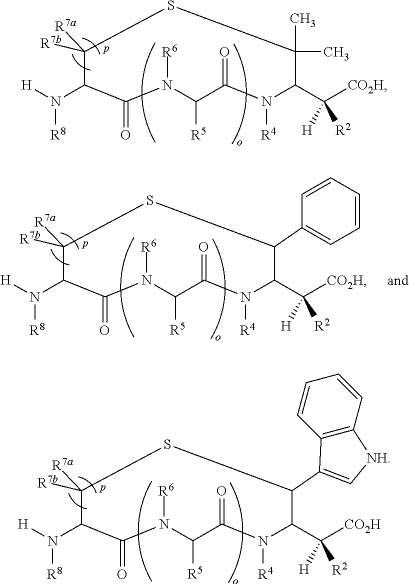

In various aspects, the thioether or selenoether compound has a structure represented by a formula:

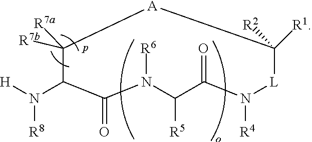

In various aspects, the thioether compound has a structure represented by a formula:

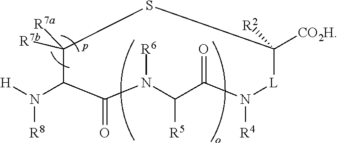

In various aspects, the thioether compound has a structure represented by a formula selected from:

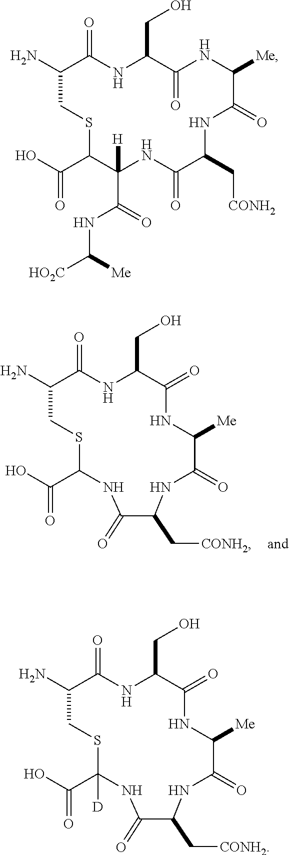 [SMILES: C[C@H](NC(=O)[C@H]1NC(=O)[C@H](CC(N)=O)NC(=O)[C@H](C)NC(=O)[C@H](CO)NC(=O)[C@@H](N)CSC1C(=O)O)C(=O)O ; C[C@@H]1NC(=O)[C@H](CO)NC(=O)[C@@H](N)CSC(C(=O)O)NC(=O)[C@H](CC(N)=O)NC1=O ; "and" ; [2H]C1(C(=O)O)NC(=O)[C@H](CC(N)=O)NC(=O)[C@H](C)NC(=O)[C@H](CO)NC(=O)[C@@H](N)CS1]

In various aspects, the thioether compound has a structure represented by a formula selected from:

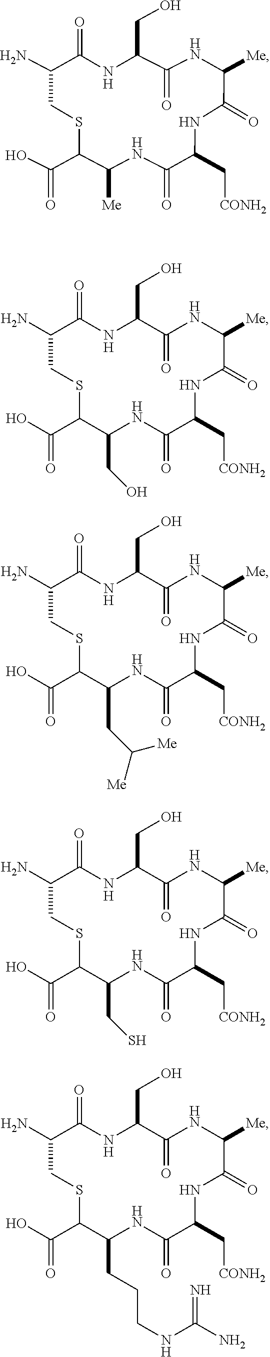

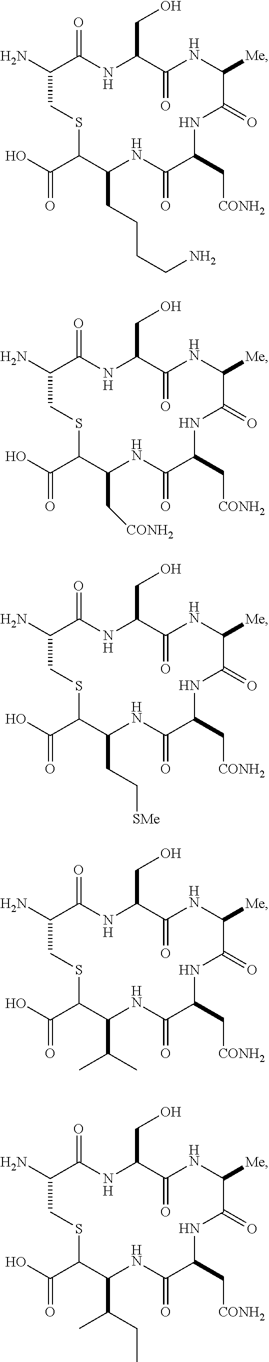

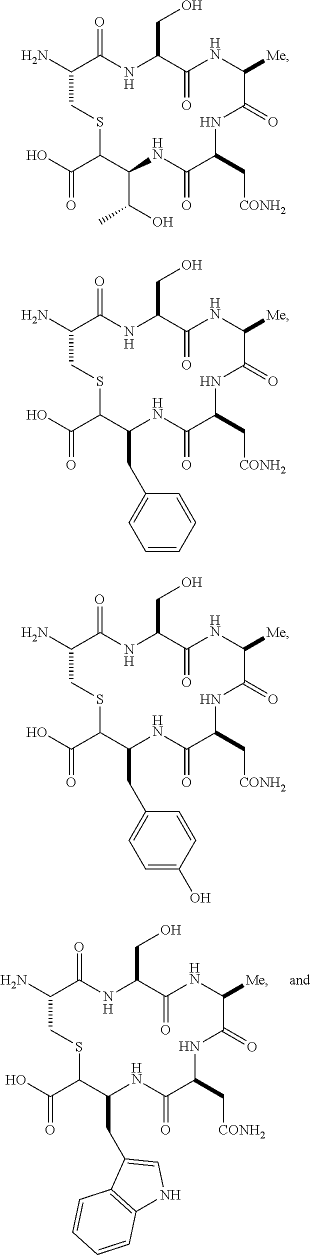

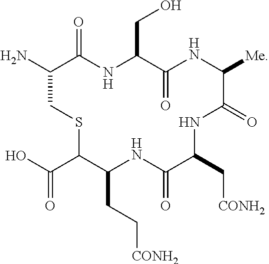 [SMILES: C[C@@H]1NC(=O)[C@H](CO)NC(=O)[C@@H](N)CSC(C(=O)O)[C@H](CCC(N)=O)NC(=O)[C@H](CC(N)=O)NC1=O]

In various aspects, the thioether compound has a structure represented by a formula selected from:

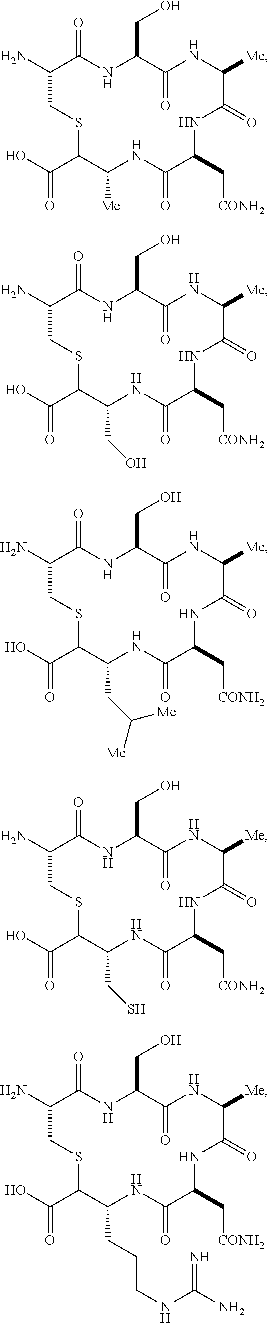

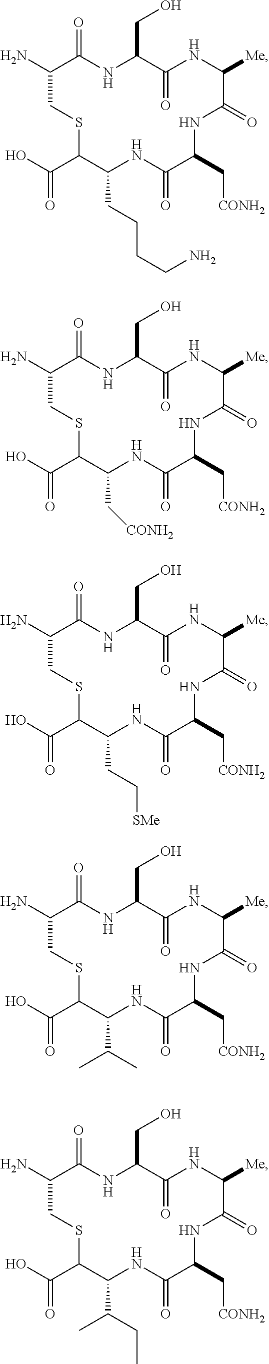

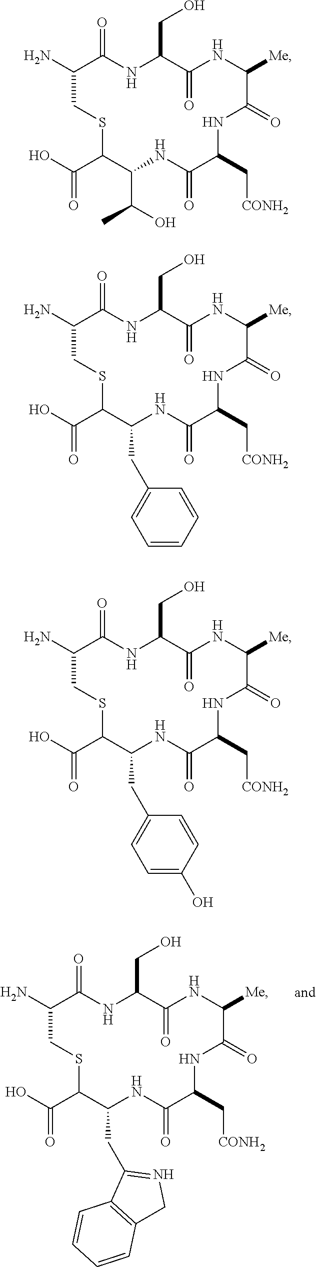

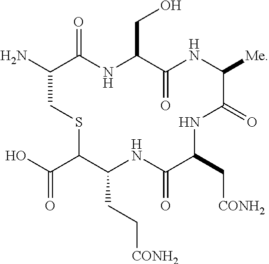 [SMILES: C[C@@H]1NC(=O)[C@H](CO)NC(=O)[C@@H](N)CSC(C(=O)O)[C@@H](CCC(N)=O)NC(=O)[C@H](CC(N)=O)NC1=O]

In various aspects, the thioether compound has a structure represented by a formula selected from:

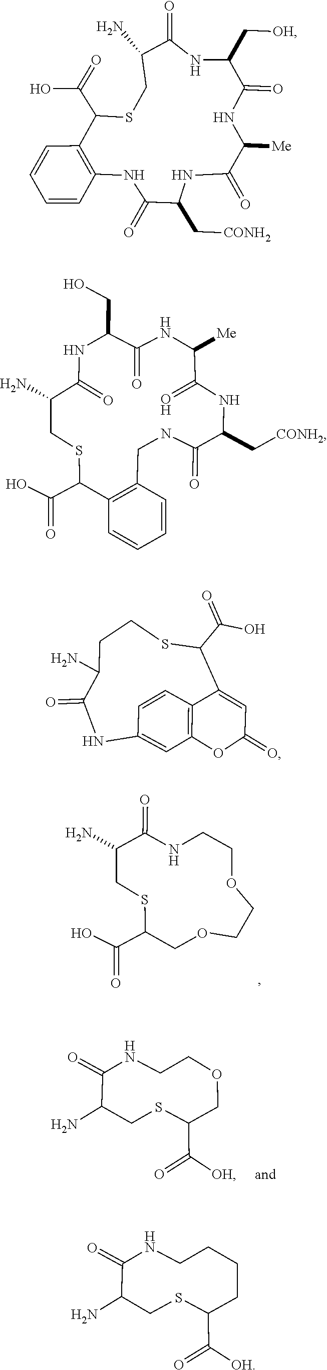 [SMILES: C[C@@H]1NC(=O)[C@H](CO)NC(=O)[C@@H](N)CSC(C(=O)O)c2ccccc2NC(=O)[C@H](CC(N)=O)NC1=O ; C[C@@H]1NC(=O)[C@H](CO)NC(=O)[C@@H](N)CSC(C(=O)O)c2ccccc2CNC(=O)[C@H](CC(N)=O)NC1=O ; NC1CCSC(C(=O)O)c2cc(=O)oc3cc(ccc23)NC1=O ; N[C@H]1CSC(C(=O)O)COCCOCCNC1=O ; NC1CSC(C(=O)O)COCCNC1=O ; NC1CSC(C(=O)O)CCCCNC1=O]

In various aspects, the thioether compound is a sactipeptide. In a further aspect, the sactipeptide has a structure represented by a formula selected from:

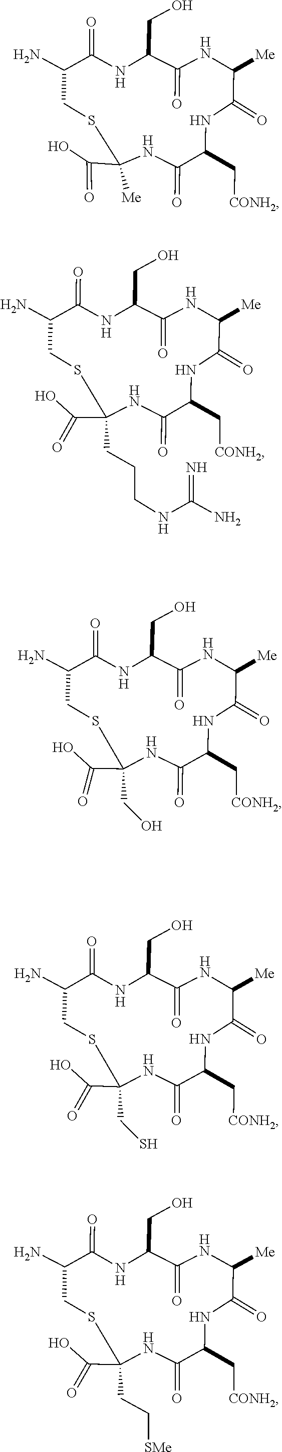 [SMILES: C[C@@H]1NC(=O)[C@H](CO)NC(=O)[C@@H](N)CS[C@](C)(C(=O)O)NC(=O)[C@H](CC(N)=O)NC1=O ; C[C@@H]1NC(=O)[C@H](CO)NC(=O)[C@@H](N)CS[C@](CCCNC(=N)N)(C(=O)O)NC(=O)[C@H](CC(N)=O)NC1=O ; C[C@@H]1NC(=O)[C@H](CO)NC(=O)[C@@H](N)CS[C@](CO)(C(=O)O)NC(=O)[C@H](CC(N)=O)NC1=O ; C[C@@H]1NC(=O)[C@H](CO)NC(=O)[C@@H](N)CS[C@](CS)(C(=O)O)NC(=O)[C@H](CC(N)=O)NC1=O ; CSCC[C@@]1(C(=O)O)NC(=O)[C@H](CC(N)=O)NC(=O)[C@H](C)NC(=O)[C@H](CO)NC(=O)[C@@H](N)CS1]

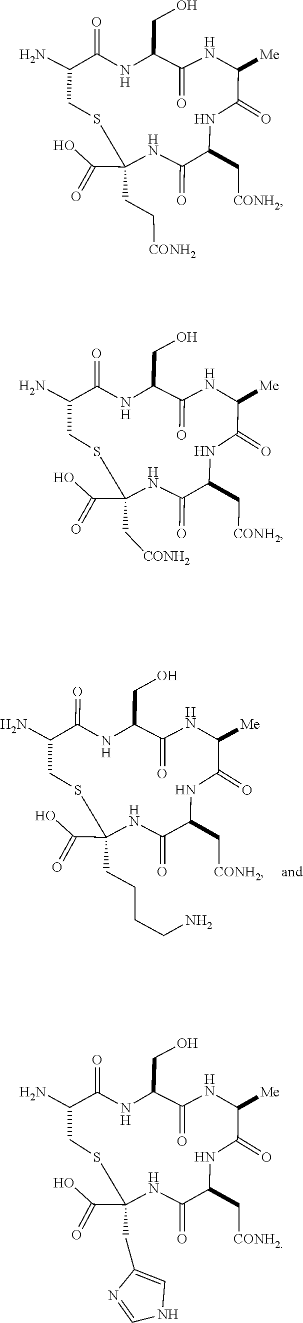 [SMILES: C[C@@H]1NC(=O)[C@H](CO)NC(=O)[C@@H](N)CS[C@](CCC(N)=O)(C(=O)O)NC(=O)[C@H](CC(N)=O)NC1=O ; C[C@@H]1NC(=O)[C@H](CO)NC(=O)[C@@H](N)CS[C@](CC(N)=O)(C(=O)O)NC(=O)[C@H](CC(N)=O)NC1=O ; C[C@@H]1NC(=O)[C@H](CO)NC(=O)[C@@H](N)CS[C@](CCCCN)(C(=O)O)NC(=O)[C@H](CC(N)=O)NC1=O ; C[C@@H]1NC(=O)[C@H](CO)NC(=O)[C@@H](N)CS[C@](Cc2c[nH]cn2)(C(=O)O)NC(=O)[C@H](CC(N)=O)NC1=O]

In various aspects, the thioether compound is a ranthipeptide. In a further aspect, the ranthipeptide has a structure represented by a formula selected from:

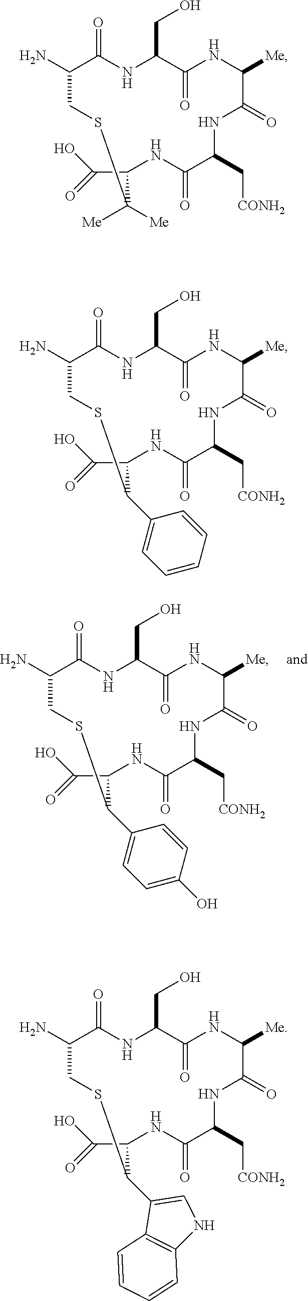

In some aspects, the compound is on the surface of the virus particles or on the tail fibers.

In some aspects, the compound is expressed as a fusion protein with a phage coat protein. In some aspects, the fusion protein comprises a full length phage coat protein.

In some aspects, the phage coat protein is a wild type protein. In some aspects, the compound is expressed on the N-terminal end of the phage coat protein. In some aspects, the compound is expressed on the C-terminal end of the phage coat protein. In some aspects, the compound is expressed within the phage coat protein.

In some aspects, the compound is attached to the phage or phage coat protein through the leader sequence or a linker. In some aspects, the linker has about 4 to about 30 residues.

In some aspects, the degree of expression of polypeptides as fusion proteins on the surface of bacteriophage particles is variable and depends, to some extent, on the size of the polypeptide. Conventional phage display systems use wild type phage coat proteins and fuse the heterologeous polypeptide (i.e. those compounds disclosed herein) to the amino terminus of the wild type amino acid sequence or an amino terminus resulting from truncation of the wild type coat protein sequence. In some aspects, the heterologous polypeptide is inserted within the phage coat protein. Segments of linker amino acids have also been added to the amino terminus of the wild type coat protein sequence to improve selection and target binding.

In some aspects, the phage or the two or more virus particles are filamentous phage. In some aspects, the filamentous phage is selected from the group consisting of M13, fl, and fd filamentous phage. In some aspects, the phage can be a filamentous phage or a lambdoid phage. Although most phage display methods have used filamentous phage, lambdoid phage display systems (WO 95/34683; U.S. Pat. No. 5,627,024), T4 phage display systems (Ren, Z-J. et al. (1998) Gene 215:439; Zhu, Z. (1997) CAN 33:534; Jiang, J. et al. (1997) can 128:44380; Ren, Z-J. et al. (1997) CAN 127:215644; Ren, Z-J. (1996) Protein Sci. 5:1833; Efunov, V. P. et al. (1995) Virus Genes 10:173) and T7 phage display systems (Smith, G. P. and Scott, J. K. (1993) Methods in Enzymology, 217, 228-257; U.S. Pat. No. 5,766,905) are also known and can be used.

In one aspect, disclosed are methods of making a phage display library comprising: (a) introducing one or more of the vectors disclosed herein and a vector comprising a phage genome into a host cell; (b) and culturing the host cells under conditions suitable to form phage particles. In some aspects, the method comprises introducing a vector comprising a nucleic acid sequence capable of encoding a compound disclosed herein and a phage coat protein. Thus, in some aspects, one vector encodes a fusion protein of one of the disclosed compounds and a phage coat protein and one vector encodes the remaining phage proteins needed to make a phage. Methods of making phage are well known in the art, thus, in some aspects any known method can be used but inserting one of the disclosed compounds herein into the vector system.

In one aspect, disclosed are methods of screening a phage display library, the method comprising: (a) introducing a disclosed vector and a vector comprising a phage genome into a host cell; (b) culturing the host cells under conditions suitable to form phage particles; (c) contacting phage particles with PapB under conditions to allow the compound to form a crosslink, thereby forming a phage particle comprising a crosslinked compound, (d) contacting the phage particles comprising the crosslinked compound with a target molecule to allow binding of the phage particle to the target molecule; (e) separating phage particles that bind to the target molecule from those that do not bind. In some aspects, once the phage particle that binds to a target molecule is identified, the protein (and nucleic acid sequence encoding the protein) that binds the target molecule can be identified. Alternatively, in some aspects, once the phage particle that binds to a target molecule is identified, the target molecule (if unknown) can be identified.

In some aspects, the methods of making a phage display and methods of screening can be combined. Thus, in some aspects, instead of starting with already produced phage, the methods of screening can first include introducing a vector comprising a nucleic acid sequence capable of encoding a compound disclosed herein and a phage coat protein and a vector comprising a phage genome into a host cell; culturing the host cells under conditions suitable to form phage particles.

In some aspects, contacting the phage particles with PapB under conditions to allow the compound to form a crosslink can be using any of the conditions described herein for crosslinking using PapB. For example, in some aspects, the conditions that allow the compound to form a crosslink with PapB include the presence of PapB, S-adenosyl-L-methionine and a reductant (i.e., sodium dithionite, titanium citrate, flavodoxin+flavodoxin reductase+NADPH, or flavin mononucleotide+NADPH). In some aspects, PapB needs to be activated with both SAM and a reductant. In some aspects, PapB can be pre-activated and then contacted with the compounds. In some aspects, the crosslinked compound is a thioether compound.

Methods of generating phage libraries and screening the libraries are also disclosed in U.S. Pat. Nos. 5,723,286; 5,432,018; 5,580,717; 5,427,908; and 5,498,530. See also U.S. Pat. Nos. 5,770,434; 5,734,018; 5,698,426; 5,763,192; and 5,723,323.

In some aspects, methods of introduce new DNA (such as vectors capable of encoding the disclosed compounds) are of great practical interest in molecular biology and modern genetic engineering. Early methods involved chemical treatment of bacteria with solutions of metal ions, generally calcium chloride, followed by heating to produce competent bacteria capable of functioning as recipient bacteria and able to take up heterologous DNA derived from a variety of sources. These early protocols provided transformation yields of about 10⁵-10⁶ transformed colonies per g of plasmid DNA. Subsequent improvements using different cations, longer treatment times and other chemical agents have allowed improvements in transformation efficiency of up to about 10⁸ colonies/g of DNA. Sambrook et al., Molecular Cloning: A Laboratory Manual, 2nd edition, (1989) Cold Spring Harbor Laboratory Press, Cold Spring Harbor, N.Y., page 1.74.

In some aspects, cells can also be transformed using high-voltage electroporation. Electroporation is suitable to introduce DNA into eukaryotic cells (e.g. animal cells, plant cells, etc.) as well as bacteria, e.g., E. coli. Sambrook et al., ibid, pages 1.75, 16.54-16.55. Different cell types require different conditions for optimal electroporation and preliminary experiments are generally conducted to find acceptable levels of expression or transformation. For mammalian cells, voltages of 250-750 V/cm result in 20-50% cell survival. An electric pulse length of 20-100 ms at a temperature ranging from room temperature to 0 °C and below using a DNA concentration of 1-40 g/mL are typical parameters. Transfection efficiency is reported to be higher using linear DNA and when the cells are suspended in buffered salt solutions than when suspended in nonionic solutions. Sambrook et al., above, pages 16.54-16.55.

Dower et al., 1988, Nucleic Acids Research, 16:6127-6145 extensively studied high-efficiency transformation of E. coli by high-voltage electroporation. This study evaluated numerous parameters, including electrical variables such as the effect of field strength and pulse length, the effect of DNA concentration and cell concentration on the recovery of transformants, accuracy, reproducibility, etc. and provided a protocol for high-efficiency electrotransformation of E. coli cells. Thus, in some aspects, the method described by Dower, incorporated by reference herein, can be used to produce the disclosed phage display libraries. See also U.S. Pat. Nos. 4,910,140 and 5,186,800 to Dower et al. and U.S. Pat. No. 4,849,355 to Wong.

In some aspects, bacteriophage (phage), such as filamentous phage, are used to create phage display libraries by transforming host cells with phage vector DNA encoding a library of peptides (e.g. the compounds disclosed here). J. K. Scott and G. P. Smith, Science, (1990), 249:386-390. In some aspects, phagemid vectors can also be used for phage display. Lowman and Wells, 1991, Methods: A Companion to Methods in Enzymology, 3:205-216. The preparation of phage and phagemid display libraries of peptides and proteins is well known in the art. These methods generally require transforming cells with phage or phagemid vector DNA to propagate the libraries as phage particles having one or more copies of the peptides or proteins displayed on the surface of the phage particles. See, for example, Barbas et al., Proc. Natl. Acad. Sci., USA, (1991), 88:7978-7982; Marks et al., J. Mol. Biol., (1991), 222:581-597; Hoogenboom and Winter, J. Mol. Biol., (1992), 227:381-388; Barbas et al., Proc. Natl. Acad. Sci., USA, (1992), 89:4457-4461; Griffiths et al., EMBO Journal, (1994), 13:3245-3260; de Kruif et al., J. Mol. Biol., (1995), 248:97-105; Bonnycastle et al., J. Mol. Biol., (1996), 258:747-762; and Vaughan et al., Nature Biotechnology (1996), 14:309-314.

In some aspects, the compounds present on the phage can have natural and/or unnatural amino acids. Methods of expressing proteins comprising one or more unnatural amino acids are well known in the art and can be used for producing phage and performing phage display. For example, Wang et al. Expanding the genetic code of Escherichia coli. Science 2001 Apr. 20; 292(5516):498-500, Anderson et al. An expanded genetic code with functional quadruplet codon, PNAS. 2004 May 18; 101(20):7566-71, and Wang et al. Genetic Incorporation of Unnatural Amino Acids into Proteins in Yeast. Methods Mol Biol. 2012; 794: 199-213, all describe methods of expressing unnatural amino acids and are incorporated by reference in their entirety herein.

In some aspects, the incorporation of unnatural amino acids can include designing an amber suppressor tRNA, generating an Aminoacyl-tRNA Synthetase (aaRS) pair, genetically encoding the Amber Suppressor, supplying the unnatural amino acid, loading the unnatural amino acid, and translation. Each of these steps is further described herein. In some aspects, when designing an amber suppressor tRNA the amber codon (UAG) can be repurposed to code for the desired unnatural amino acid. In some aspects, a tRNA can be engineered that can recognize the amber codon and carry the unnatural amino acid. This tRNA can be referred to as an “amber suppressor tRNA.” In some aspects, when generating an aminoacyl-tRNA synthetase (aaRS) pair a specialized aminoacyl-tRNA synthetase (aaRS) is designed or evolved to specifically recognize and load the unnatural amino acid onto the amber suppressor tRNA. In some aspects, this aaRS can have high specificity for the unnatural amino acid to minimize errors in translation. In some aspects, genetically encoding the amber suppressor comprises integrating the designed amber suppressor into the host organism's genome (requires another plasmid . . . ). This can allow the engineered tRNA to be transcribed and translated like any other cellular RNA. In some aspects, supplying the unnatural amino acid involves providing the unnatural amino acids, which are not naturally found in cells, exogenously. In some aspects they can be chemically synthesized or obtained through other biotechnological processes. These amino acids can be paired to the aminoacyl-tRNA synthetase. In some aspects, the unnatural amino acid can be loaded onto the amber suppressor tRNA by the designed aaRS. In some aspects, the aaRS specifically recognizes the unnatural amino acid and catalyzes its attachment to the tRNA, creating an aminoacyl-tRNA complex. In some aspects, translation, which occurs during protein synthesis, occurs when the ribosome encounters the amber codon (UAG) in the mRNA and the engineered amber suppressor tRNA, carrying the unnatural amino acid, is brought into the ribosome's A-site. In some aspects, this tRNA can effectively suppress the stop codon by inserting the unnatural amino acid into the growing polypeptide chain. This can be used with other codons as well, the amber codon is the most commonly used.

2. Ribosome or mRNA Display Libraries

In some aspects, one or more of the disclosed compounds can be displayed on a ribosome complex comprising a ribosome, a mRNA molecule, wherein the mRNA molecule comprises a nucleotide sequence capable of encoding one or more of the compounds disclosed herein and lacking an in-frame stop codon; and one or more of the compounds disclosed herein.

In one aspect, disclosed are ribosome display libraries comprising a plurality of complexes, wherein each complex comprises: (a) a mRNA molecule, wherein the mRNA molecule comprises a nucleotide sequence capable of encoding one or more of the compounds disclosed herein and lacking an in-frame stop codon; and (b) one or more of the compounds or thioether or selenoether compounds disclosed herein. In some aspects, each complex further comprises a ribosome.

In various aspects, the compound has a structure represented by a formula:

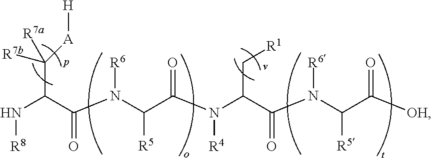

wherein o is 0, 1, 2, 3, 4, 5, 6, 7, 8, or 9; wherein p is 1 or 2; wherein t is an integer from 0 to 500; wherein v is 1, 2, 3, 4, or 5; wherein A is S or Se; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

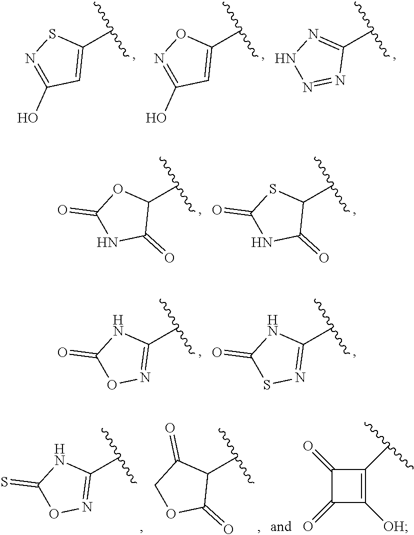

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, provided that the compound is not PapA.

In various aspects, the compound has a structure represented by a formula:

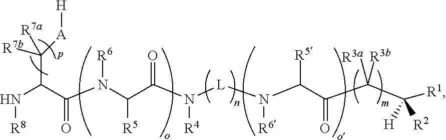

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein A is S or Se; wherein L, when present, is selected from C2-C4 alkyl, —(C1-C4 alkyl)(OCH₂CH₂)_{q}, and a structure selected from:

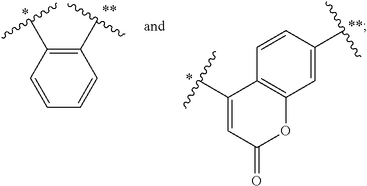

wherein q is 1, 2, 3, or 4; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

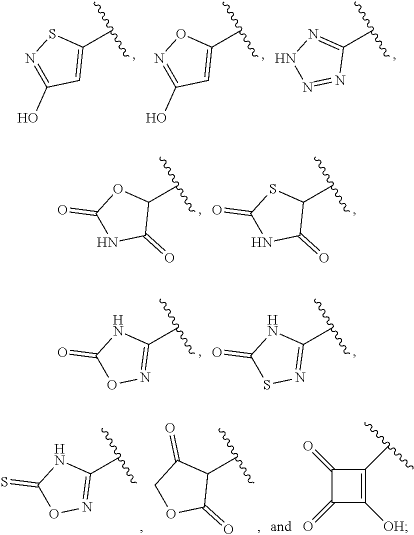

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, provided that the compound is not PapA.

In various aspects, the thioether or selenoether compound has a structure represented by a formula:

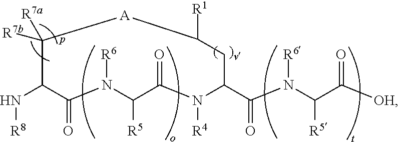

wherein o is 0, 1, 2, 3, 4, 5, 6, 7, 8, or 9; wherein p is 1 or 2; wherein t is an integer from 0 to 500; wherein v′ is 0, 1, 2, 3, or 4; wherein A is S or Se; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

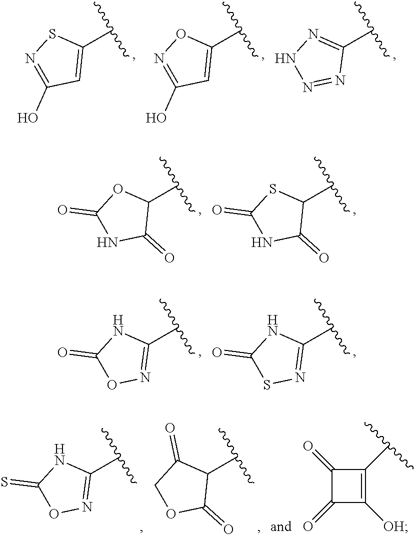

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl.

In various aspects, the thioether or selenoether compounds has a structure represented by a formula:

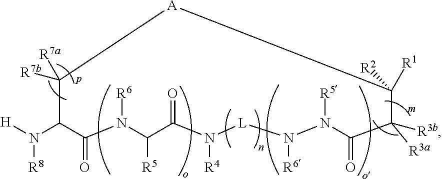

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein A is S or Se; wherein L, when present, is selected from C2-C4 alkyl, —(C1-C4 alkyl)(OCH₂CH₂)_{q}, and a structure selected from:

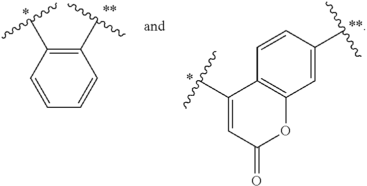

wherein q is 1, 2, 3, or 4; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

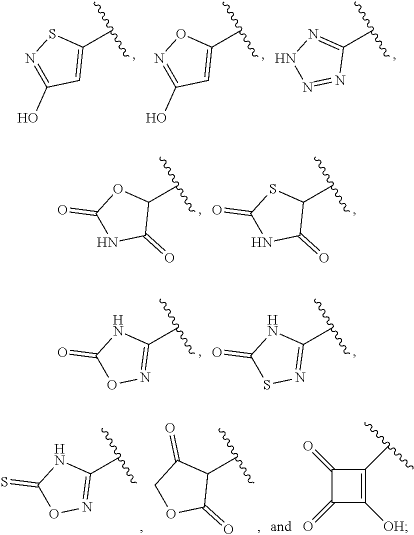

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4.

In various aspects, o is independently 0, 1, 2, 3, 4, 5, 6, or 7.

In various aspects, t is 0.

In various aspects, v is 1 or 2.

In various aspects, w is 0 or 1.

In various aspects, R¹ is —CO₂H or a structure:

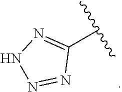 [SMILES: *c1nn[nH]n1]

In various aspects, R¹ is —CO₂H.

In various aspects, the compound comprises one or more D-amino acid residues.

In various aspects, the compound comprises one or more p-amino acid residues.

In various aspects, the compound comprises one or more N-methylated amino acid residues.

In various aspects, the compound has a structure represented by a formula:

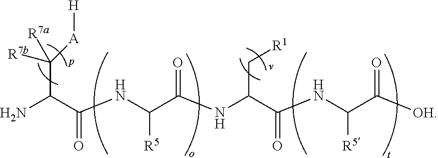

In various aspects, the compound has a structure represented by a formula:

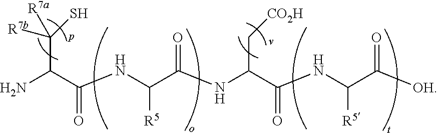

In various aspects, the method produces a thioether or selenoether compound has a structure represented by a formula:

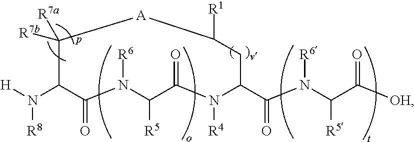

wherein v′ is 0, 1, 2, or 3.

In various aspects, m is 0. In a further aspect, m is 1.

In various aspects, n is 0. In a further aspect, n is 1.

In various aspects, o is 0, 1, 2, 3, 4, 5, 6, or 7. In a further aspect, o is 1, 2, 3, 4, 5, 6, 7, 8, or 9. In a still further aspect, o is 1, 2, 3, or 4.

In various aspects, p is 1. In a further aspect, p is 2.

In various aspects, w is 0. In a further aspect, w is 1.

In various aspects, A is S. In a further aspect, A is Se.

In various aspects, L is C2-C4 alkyl. In a further aspect, L is —(C1-C4 alkyl)(OCH₂CH₂)_{q}. In a still further aspect, L is a structure selected from:

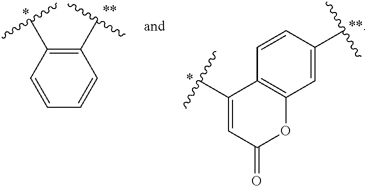 [SMILES: *c1ccccc1* ; *c1ccc2c(*)cc(=O)oc2c1]

In various aspects, R¹ is selected from —CO₂H and a structure:

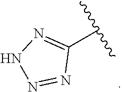 [SMILES: *c1nn[nH]n1]

In various aspects, R¹ is —CO₂H.

In various aspects, R² is a residue of a side chain of an amino acid selected from alanine, valine, leucine, serine, cysteine, methionine, arginine, lysine, asparagine, glycine, phenylalanine, tyrosine, and tryptophan. In a further aspect, R² is a residue of a side chain of an amino acid selected from alanine, leucine, serine, cysteine, methionine, arginine, lysine, asparagine, and glycine.

In various aspects, one of R^{3a} and R^{3b}, when present, is hydrogen, and one of R^{3a} and R^{3b}, when present, is a residue of a side chain of an amino acid selected from alanine, valine, leucine, serine, cysteine, methionine, arginine, lysine, asparagine, glycine, phenylalanine, tyrosine, and tryptophan.

In various aspects, R⁴ is hydrogen. In a further aspect, R⁴ is methyl.

In various aspects, each occurrence of R⁵, when present, is independently a residue of a side chain of an amino acid selected from alanine, valine, leucine, serine, cysteine, methionine, arginine, lysine, asparagine, glycine, phenylalanine, tyrosine, and tryptophan.

In various aspects, each occurrence of R⁶, when present, is hydrogen. In a further aspect, each occurrence of R⁶, when present, is methyl.

In various aspects, each of R^{7a} and R^{7b}, when present, is hydrogen. In a further aspect, each of R^{7a} and R^{7b}, when present, is methyl.

In various aspects, the compound has a structure represented by a formula:

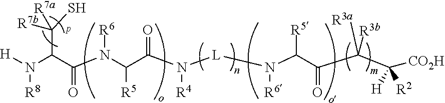

In various aspects, the compound has a structure represented by a formula:

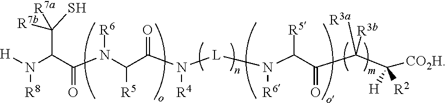

In various aspects, the compound has a structure represented by a formula:

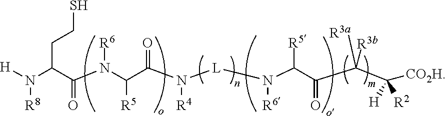

In various aspects, the compound has a structure represented by a formula:

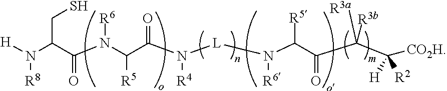

In various aspects, the compound has a structure represented by a formula:

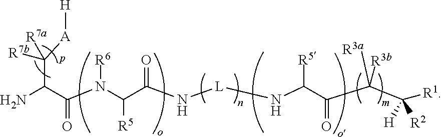

In various aspects, the compound has a structure represented by a formula:

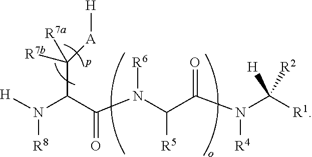

In various aspects, the compound has a structure represented by a formula:

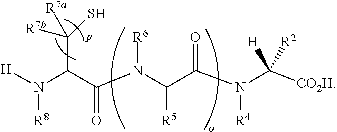

In various aspects, the compound has a structure represented by a formula:

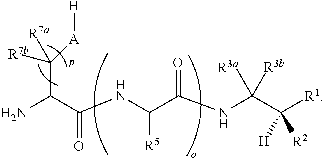

In a further aspect, o is 1, 2, 3, 4, 5, 6, 7, 8, or 9. In a still further aspect, one of R^{3a} and R^{3b} when present, is hydrogen, and one of R^{3a} and R^{3b}, when present, is a residue of a side chain of an amino acid selected from alanine, valine, leucine, serine, cysteine, methionine, arginine, lysine, asparagine, glycine, phenylalanine, tyrosine, and tryptophan.

In various aspects, the compound has a structure represented by a formula:

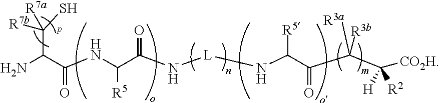

In various aspects, the compound has a structure represented by a formula:

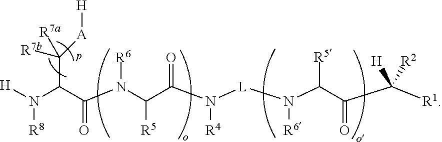

In various aspects, the compound has a structure represented by a formula:

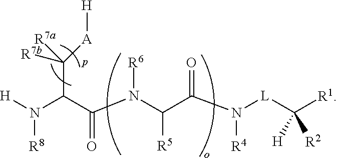

In various aspects, the compound has a structure represented by a formula:

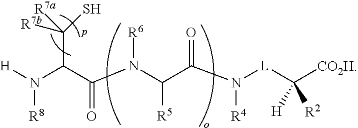

In a further aspect, o is 1, 2, 3, 4, 5, 6, 7, 8, or 9.

In various aspects, the compound has a structure represented by a formula:

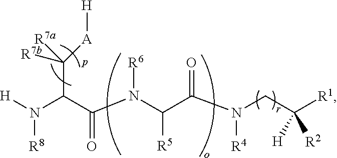

wherein r is 2, 3, or 4.

In various aspects, the compound has a structure represented by a formula:

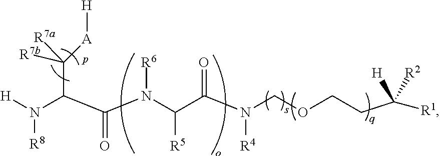

wherein s is 1 or 2.

In various aspects, the compound has a structure represented by a formula:

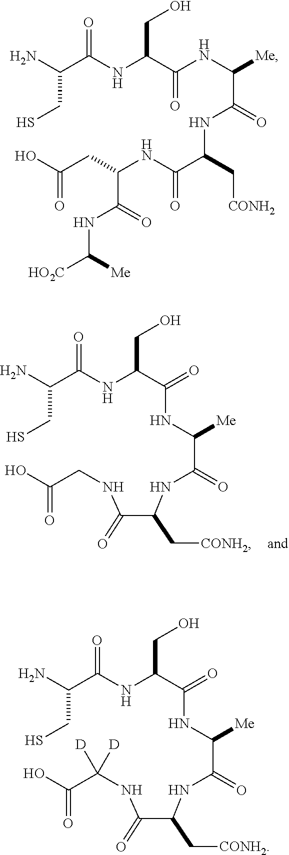 [SMILES: C[C@H](NC(=O)[C@H](CC(=O)O)NC(=O)[C@H](CC(N)=O)NC(=O)[C@H](C)NC(=O)[C@H](CO)NC(=O)[C@@H](N)CS)C(=O)O ; C[C@H](NC(=O)[C@H](CO)NC(=O)[C@@H](N)CS)C(=O)N[C@@H](CC(N)=O)C(=O)NCC(=O)O ; "and" ; [2H]C([2H])(NC(=O)[C@H](CC(N)=O)NC(=O)[C@H](C)NC(=O)[C@H](CO)NC(=O)[C@@H](N)CS)C(=O)O]

In various aspects, the thioether compound has a structure represented by a formula:

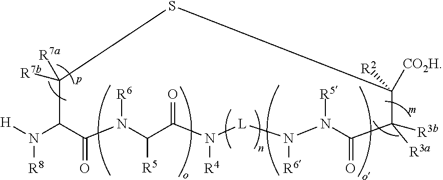

In various aspects, the thioether or selenoether compound has a structure represented by a formula:

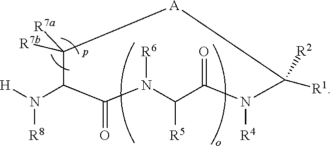

In various aspects, the thioether compound has a structure represented by a formula:

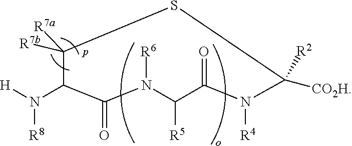

In various aspects, the thioether or selenoether compound has a structure represented by a formula:

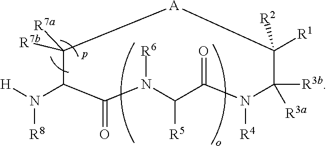

In various aspects, the thioether or selenoether compound has a structure represented by a formula:

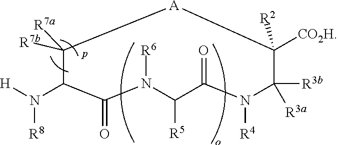

In various aspects, the thioether or selenoether compound has a structure represented by a formula:

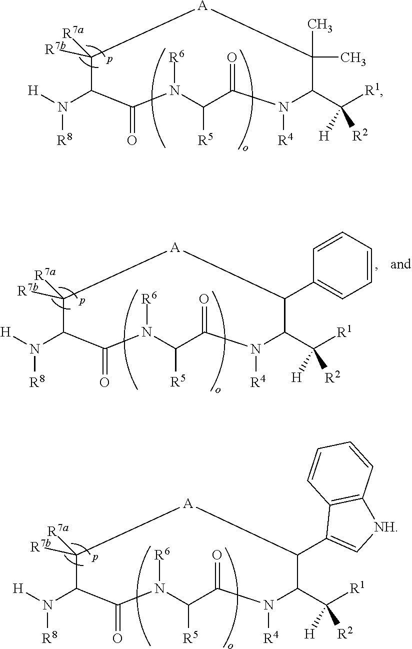

In various aspects, the thioether compound has a structure represented by a formula:

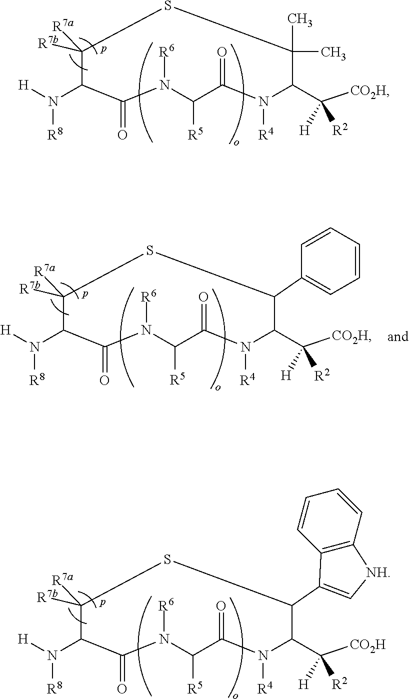

In various aspects, the thioether or selenoether compound has a structure represented by a formula:

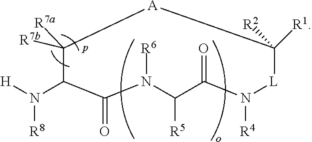

In various aspects, the thioether compound has a structure represented by a formula:

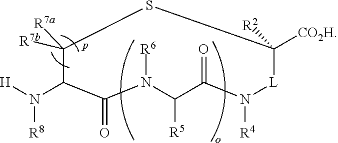

In various aspects, the thioether compound has a structure represented by a formula selected from:

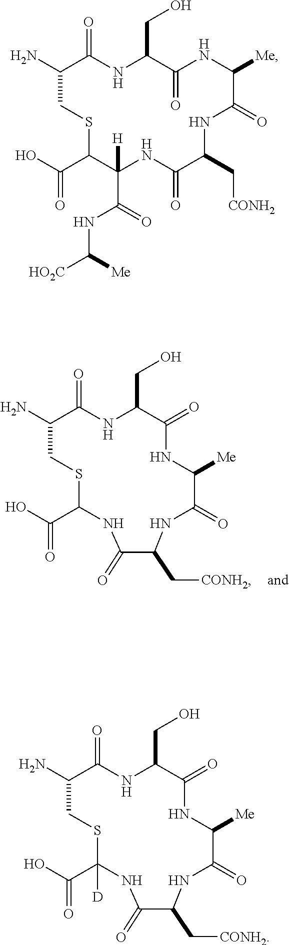 [SMILES: C[C@H](NC(=O)[C@H]1NC(=O)[C@H](CC(N)=O)NC(=O)[C@H](C)NC(=O)[C@H](CO)NC(=O)[C@@H](N)CSC1C(=O)O)C(=O)O ; C[C@@H]1NC(=O)[C@H](CO)NC(=O)[C@@H](N)CSC(C(=O)O)NC(=O)[C@H](CC(N)=O)NC1=O ; "and" ; [2H]C1(C(=O)O)NC(=O)[C@H](CC(N)=O)NC(=O)[C@H](C)NC(=O)[C@H](CO)NC(=O)[C@@H](N)CS1]

In various aspects, the thioether compound has a structure represented by a

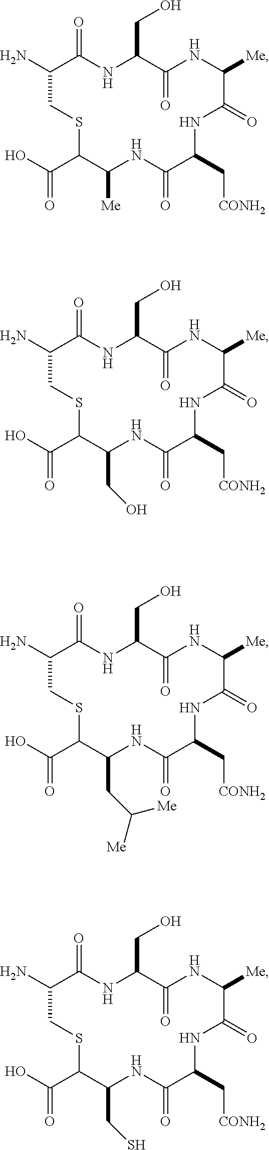 [SMILES: C[C@@H]1NC(=O)[C@H](CO)NC(=O)[C@@H](N)CSC(C(=O)O)[C@H](C)NC(=O)[C@H](CC(N)=O)NC1=O ; C[C@@H]1NC(=O)[C@H](CO)NC(=O)[C@@H](N)CSC(C(=O)O)[C@H](CO)NC(=O)[C@H](CC(N)=O)NC1=O ; CC(C)C[C@@H]1NC(=O)[C@H](CC(N)=O)NC(=O)[C@H](C)NC(=O)[C@H](CO)NC(=O)[C@@H](N)CSC1C(=O)O ; C[C@@H]1NC(=O)[C@H](CO)NC(=O)[C@@H](N)CSC(C(=O)O)[C@H](CS)NC(=O)[C@H](CC(N)=O)NC1=O]

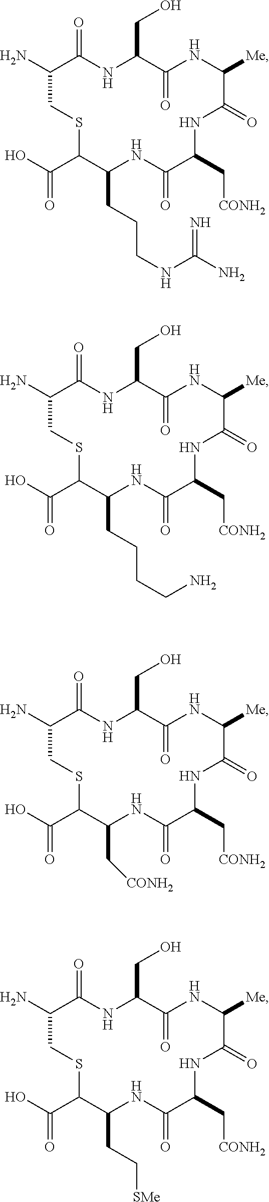 [SMILES: C[C@@H]1NC(=O)[C@H](CO)NC(=O)[C@@H](N)CSC(C(=O)O)[C@H](CCCNC(=N)N)NC(=O)[C@H](CC(N)=O)NC1=O ; C[C@@H]1NC(=O)[C@H](CO)NC(=O)[C@@H](N)CSC(C(=O)O)[C@H](CCCCN)NC(=O)[C@H](CC(N)=O)NC1=O ; C[C@@H]1NC(=O)[C@H](CO)NC(=O)[C@@H](N)CSC(C(=O)O)[C@H](CC(N)=O)NC(=O)[C@H](CC(N)=O)NC1=O ; CSCC[C@@H]1NC(=O)[C@H](CC(N)=O)NC(=O)[C@H](C)NC(=O)[C@H](CO)NC(=O)[C@@H](N)CSC1C(=O)O]

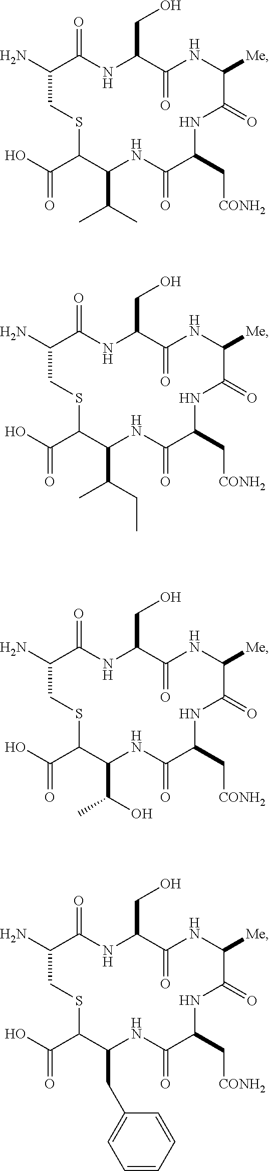 [SMILES: CC(C)[C@@H]1NC(=O)[C@H](CC(N)=O)NC(=O)[C@H](C)NC(=O)[C@H](CO)NC(=O)[C@@H](N)CSC1C(=O)O ; CCC(C)[C@@H]1NC(=O)[C@H](CC(N)=O)NC(=O)[C@H](C)NC(=O)[C@H](CO)NC(=O)[C@@H](N)CSC1C(=O)O ; C[C@@H]1NC(=O)[C@H](CO)NC(=O)[C@@H](N)CSC(C(=O)O)[C@H]([C@@H](C)O)NC(=O)[C@H](CC(N)=O)NC1=O ; C[C@@H]1NC(=O)[C@H](CO)NC(=O)[C@@H](N)CSC(C(=O)O)[C@H](Cc2ccccc2)NC(=O)[C@H](CC(N)=O)NC1=O]

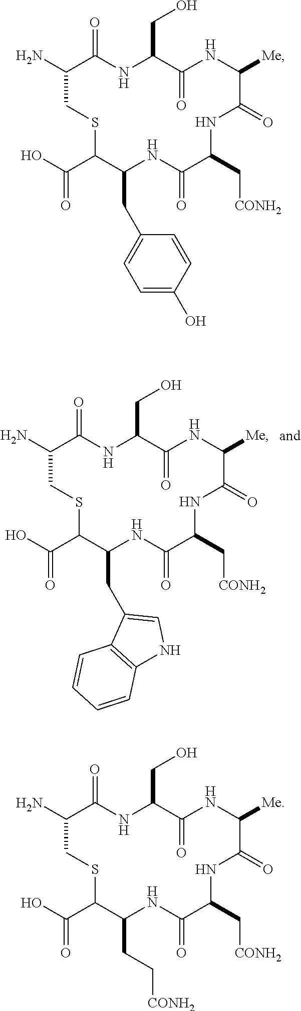 [SMILES: C[C@@H]1NC(=O)[C@H](CO)NC(=O)[C@@H](N)CSC(C(=O)O)[C@H](Cc2ccc(O)cc2)NC(=O)[C@H](CC(N)=O)NC1=O ; C[C@@H]1NC(=O)[C@H](CO)NC(=O)[C@@H](N)CSC(C(=O)O)[C@H](Cc2c[nH]c3ccccc23)NC(=O)[C@H](CC(N)=O)NC1=O ; "and" ; C[C@@H]1NC(=O)[C@H](CO)NC(=O)[C@@H](N)CSC(C(=O)O)[C@H](CCC(N)=O)NC(=O)[C@H](CC(N)=O)NC1=O]

In various aspects, the thioether compound has a structure represented by a

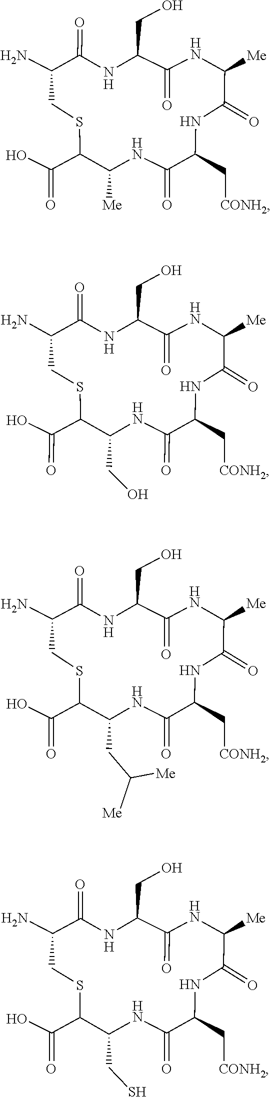 [SMILES: C[C@@H]1NC(=O)[C@H](CO)NC(=O)[C@@H](N)CSC(C(=O)O)[C@@H](C)NC(=O)[C@H](CC(N)=O)NC1=O ; C[C@@H]1NC(=O)[C@H](CO)NC(=O)[C@@H](N)CSC(C(=O)O)[C@@H](CO)NC(=O)[C@H](CC(N)=O)NC1=O ; CC(C)C[C@H]1NC(=O)[C@H](CC(N)=O)NC(=O)[C@H](C)NC(=O)[C@H](CO)NC(=O)[C@@H](N)CSC1C(=O)O ; C[C@@H]1NC(=O)[C@H](CO)NC(=O)[C@@H](N)CSC(C(=O)O)[C@@H](CS)NC(=O)[C@H](CC(N)=O)NC1=O]

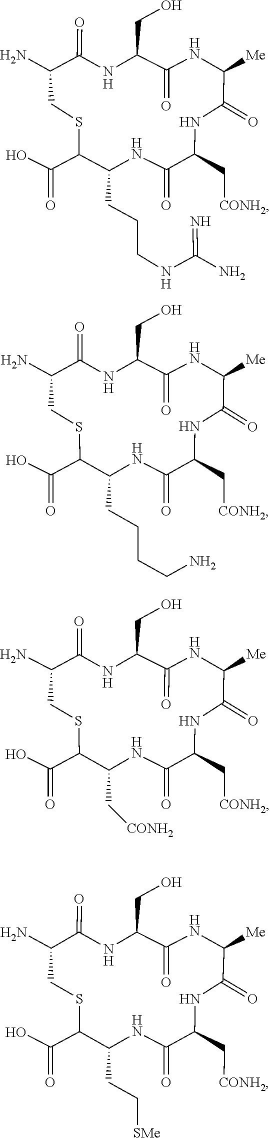

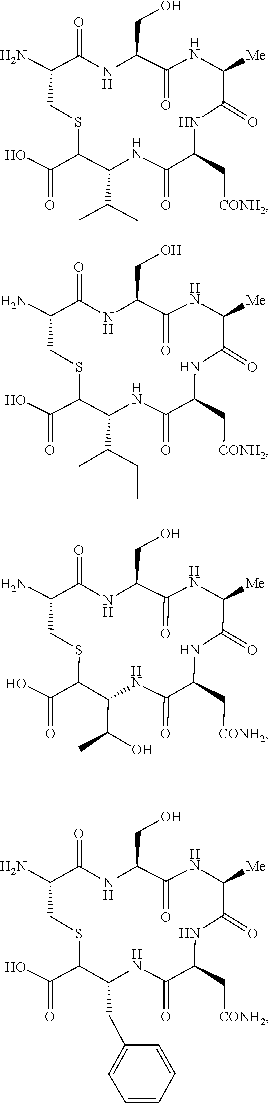

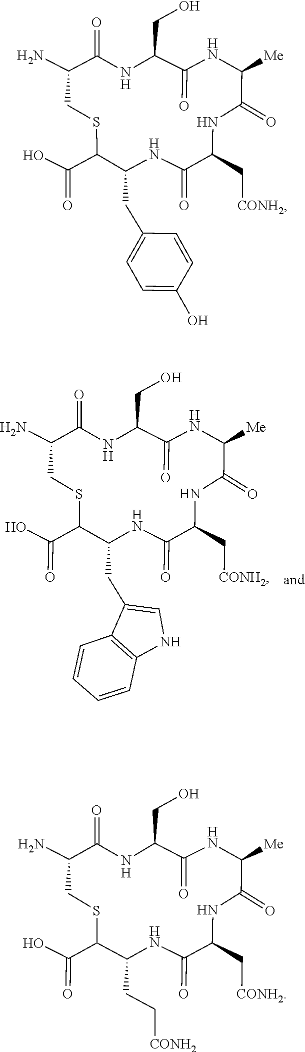 [SMILES: C[C@@H]1NC(=O)[C@H](CO)NC(=O)[C@@H](N)CSC(C(=O)O)[C@@H](Cc2ccc(O)cc2)NC(=O)[C@H](CC(N)=O)NC1=O ; C[C@@H]1NC(=O)[C@H](CO)NC(=O)[C@@H](N)CSC(C(=O)O)[C@@H](Cc2c[nH]c3ccccc23)NC(=O)[C@H](CC(N)=O)NC1=O ; C[C@@H]1NC(=O)[C@H](CO)NC(=O)[C@@H](N)CSC(C(=O)O)[C@@H](CCC(N)=O)NC(=O)[C@H](CC(N)=O)NC1=O]

In various aspects, the thioether compound has a structure represented by a formula selected from:

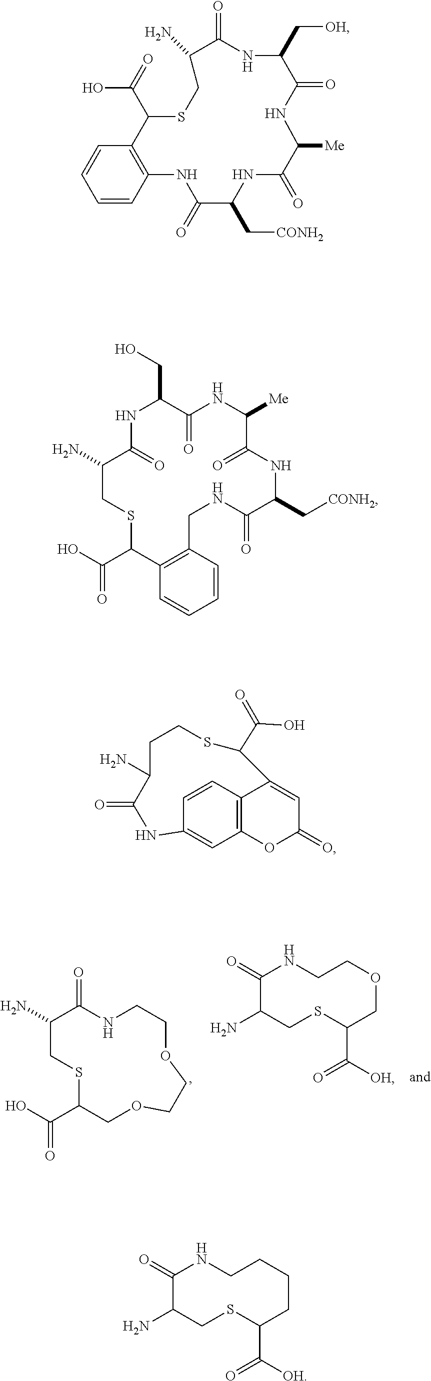 [SMILES: C[C@@H]1NC(=O)[C@H](CO)NC(=O)[C@@H](N)CSC(C(=O)O)c2ccccc2NC(=O)[C@H](CC(N)=O)NC1=O ; C[C@@H]1NC(=O)[C@H](CO)NC(=O)[C@@H](N)CSC(C(=O)O)c2ccccc2CNC(=O)[C@H](CC(N)=O)NC1=O ; NC1CCSC(C(=O)O)c2cc(=O)oc3cc(ccc23)NC1=O ; N[C@H]1CSC(C(=O)O)COCCOCCNC1=O ; NC1CSC(C(=O)O)COCCNC1=O ; NC1CSC(C(=O)O)CCCCNC1=O]

In various aspects, the thioether compound is a sactipeptide. In a further aspect, the sactipeptide has a structure represented by a formula selected from:

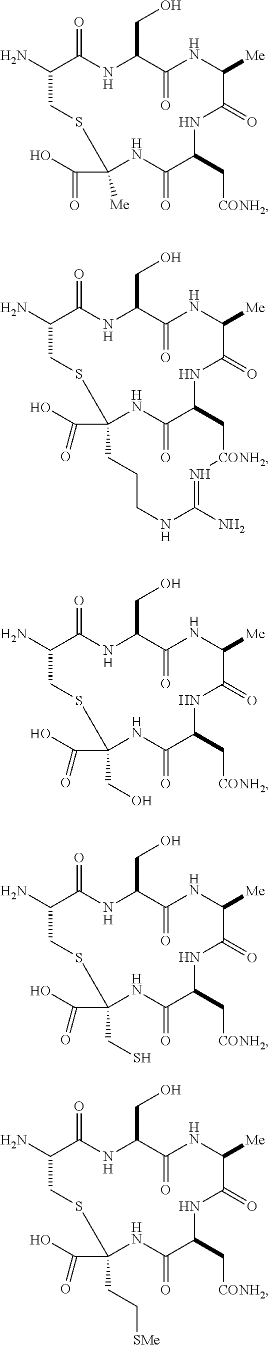

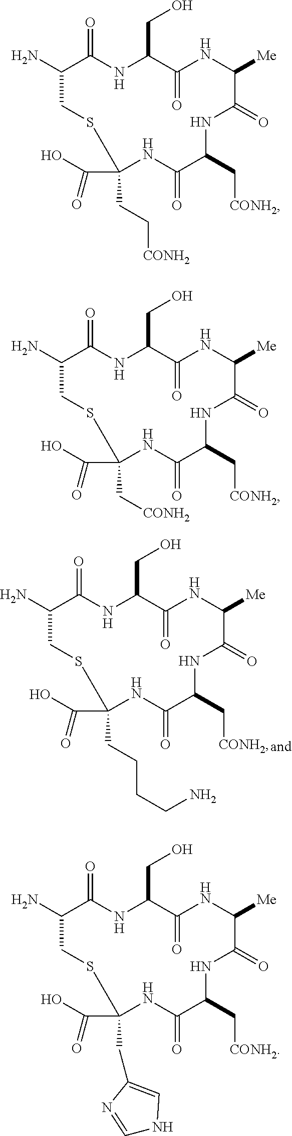

In various aspects, the thioether compound is a ranthipeptide. In a further aspect, the ranthipeptide has a structure represented by a formula selected from:

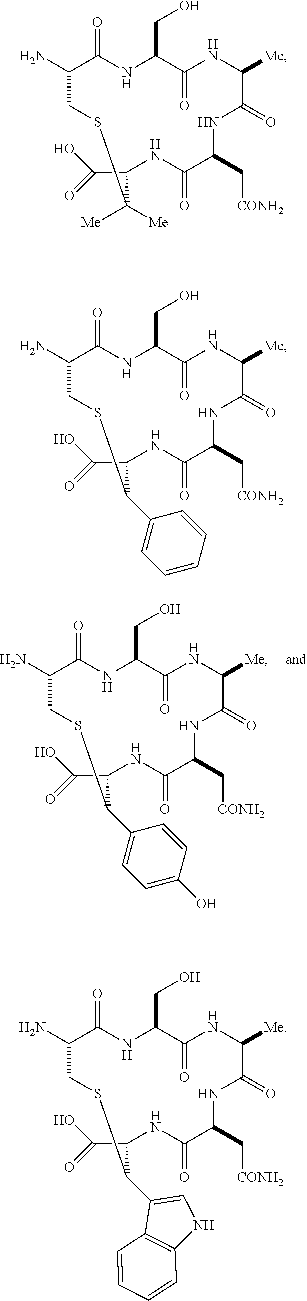

In some aspects, these complexes can occur during translation. Because there is no in-frame stop codon, the mRNA and the translated protein can remain intact with the ribosome.

In one aspect, disclosed are methods of screening using a ribosome display library comprising: (a) obtaining a plurality of mRNA molecules, each mRNA molecule comprising a nucleotide sequence capable of encoding one or more of the compounds disclosed herein and lacking an in-frame stop codon; (b) incubating the plurality of mRNA molecules under conditions for ribosome translation of the mRNA molecules to produce encoded compounds, whereby complexes each comprising the mRNA molecule that encodes a compound, the encoded compound, and a ribosome are formed; (c) contacting the complexes with PapB under conditions to allow the compound to form a crosslink, thereby forming a complex comprising a crosslinked compound (e.g. thioether compound), (d) contacting the complexes with a ligand that binds the compound, (e) selecting one or more complexes each displaying a compound able to bind the ligand; (f) isolating the mRNA molecule from the complex bound to the ligand; and (g) reverse transcribing the mRNA molecule to cDNA.

In some aspects, contacting the complexes with PapB under conditions to allow the compound to form a crosslink can be using any of the conditions described herein for crosslinking using PapB. For example, in some aspects, the conditions that allow the compound to form a crosslink with PapB include the presence of PapB, S-adenosyl-L-methionine and a reductant (i.e., sodium dithionite, titanium citrate, flavodoxin+flavodoxin reductase+NADPH, or flavin mononucleotide+NADPH). In some aspects, PapB needs to be activated with both SAM and a reductant. In some aspects, PapB can be pre-activated and then contacted with the compounds.

In some aspects, ribosome or mRNA display can be used to screen the disclosed compounds. Ribosome (or polysome) or mRNA display and selection involves construction of nucleic acid libraries, screening for binding, and identification of binding entities of interest. The library is made by synthesizing a DNA pool of diverse sequences (e.g. those that encode the disclosed compounds) that are then transcribed to produce a pool of mRNAs. In vitro translation is used to generate the encoded polypeptides or proteins displayed, and desirable binding interactions are selected using immobilised targets. mRNA encoding the binding entities can be used to make cDNA, which can then be amplified and the process may be repeated to enrich the population for genes encoding binders. The selected proteins may later be identified by cloning individual coding sequences and DNA sequencing. The technology has been reviewed extensively (Hanes et al., (2000) Meth. Enzymol 328, 403-430/Plickthun et al., (2000) Adv. Prot. Chem. 55, 367-403; Lipovsek and Plickthun (2004) J. Immunological Methods 290, 51-67).

In some aspects, the translated protein remains connected to the ribosome and to its encoding mRNA (due to the lack of an in-frame stop codon). Therefore, upon binding of the protein to a target, the mRNA that encodes that protein can easily be obtained.

In some aspects, mRNA display is similar to ribosome display in that the mRNA is complexed with the translated protein, but mRNA display uses a linkage such as a small adaptor molecule (e.g. puromycin). Thus, similar to the ribosome display, if there is no stop codon, when the ribosome reaches the end of the mRNA, pruomycin enters the peptidyltransferase site to form a covalent bond with the nasent peptide chain and makes a protein-mRNA fusion.

In some aspects, these screens can result in the nucleic acid sequence that encodes a protein that binds to a known target. The screens can also result in identifying a target that binds to a known protein.

### 3. Cell-Surface Display Libraries

In one aspect, disclosed are cell-surface display libraries comprising a host cell, wherein the host cell comprises one or more of the compounds or thioether or selenoether compounds disclosed herein on the cell surface.

In various aspects, the compound has a structure represented by a formula:

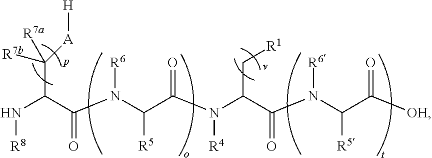

wherein o is 0, 1, 2, 3, 4, 5, 6, 7, 8, or 9; wherein p is 1 or 2; wherein t is an integer from 0 to 500; wherein v is 1, 2, 3, 4, or 5; wherein A is S or Se; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

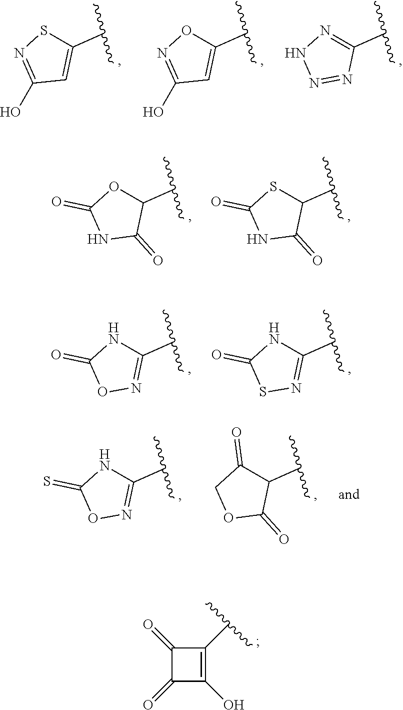

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, provided that the compound is not PapA.

In various aspects, the compound has a structure represented by a formula:

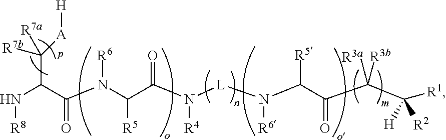

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein A is S or Se; wherein L, when present, is selected from C2-C4 alkyl, —(C1-C4 alkyl)(OCH₂CH₂)_{q}, and a structure selected from:

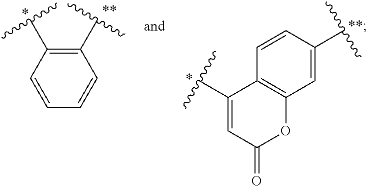

wherein q is 1, 2, 3, or 4; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

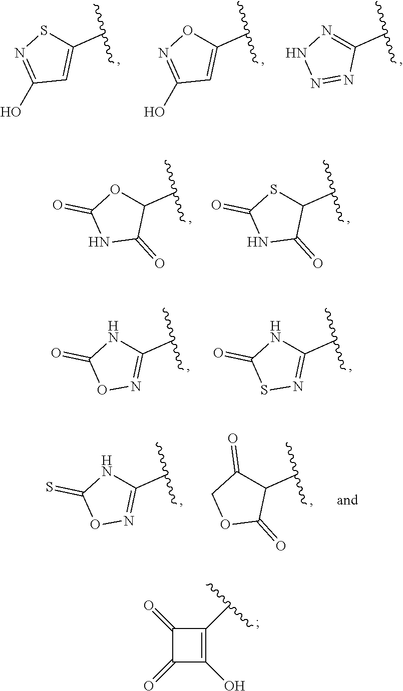

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, provided that the compound is not PapA.

In various aspects, the thioether or selenoether compound has a structure represented by a formula:

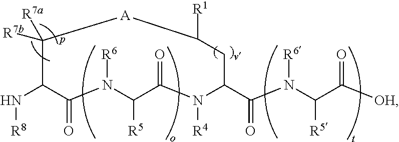

wherein o is 0, 1, 2, 3, 4, 5, 6, 7, 8, or 9; wherein p is 1 or 2; wherein t is an integer from 0 to 500; wherein v′ is 0, 1, 2, 3, or 4; wherein A is S or Se; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

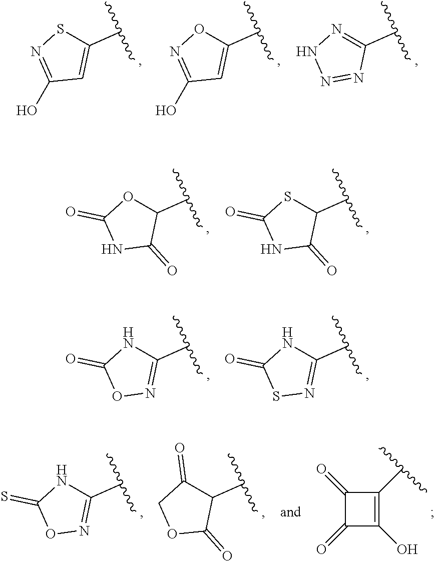

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl.

In various aspects, the thioether or selenoether compound has a structure represented by a formula:

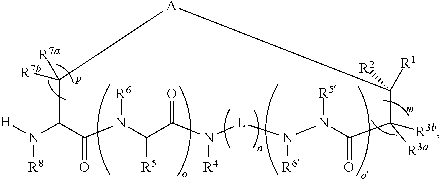

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein A is S or Se; wherein L, when present, is selected from C2-C4 alkyl, —(C1-C4 alkyl)(OCH₂CH₂)_{q}, and a structure selected from:

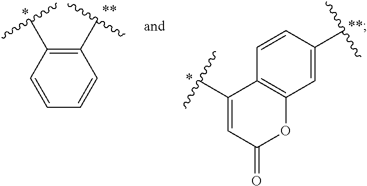

wherein q is 1, 2, 3, or 4; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

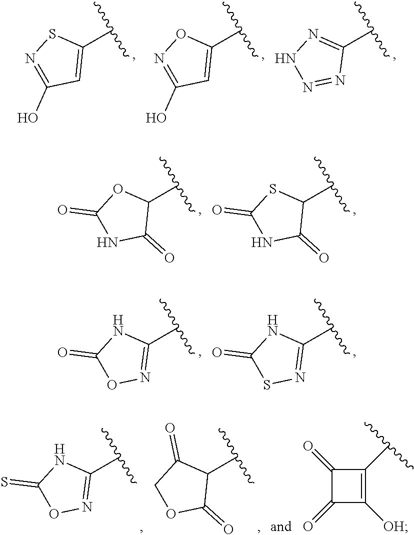

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl.

In various aspects, o is independently 0, 1, 2, 3, 4, 5, 6, or 7.

In various aspects, t is 0.

In various aspects, v is 1 or 2.

In various aspects, w is 0 or 1.

In various aspects, R¹ is —CO₂H or a structure:

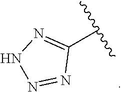 [SMILES: *c1nn[nH]n1]

In various aspects, R¹ is —CO₂H.

In various aspects, the compound comprises one or more D-amino acid residues.

In various aspects, the compound comprises one or more p-amino acid residues.

In various aspects, the compound comprises one or more N-methylated amino acid residues.

In various aspects, the compound has a structure represented by a formula:

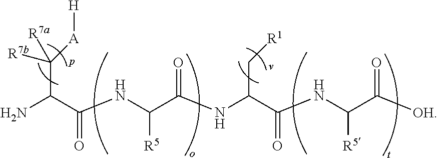

In various aspects, the compound has a structure represented by a formula:

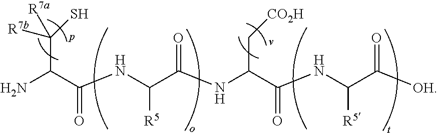

In various aspects, the method produces a thioether or selenoether compound has a structure represented by a formula:

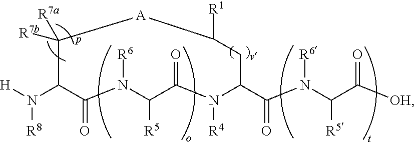

wherein v′ is 0, 1, 2, or 3.

In various aspects, m is 0. In a further aspect, m is 1.

In various aspects, n is 0. In a further aspect, n is 1.

In various aspects, o is 0, 1, 2, 3, 4, 5, 6, or 7. In a further aspect, o is 1, 2, 3, 4, 5, 6, 7, 8, or 9. In a still further aspect, o is 1, 2, 3, or 4.

In various aspects, p is 1. In a further aspect, p is 2.

In various aspects, w is 0. In a further aspect, w is 1.

In various aspects, A is S. In a further aspect, A is Se.

In various aspects, L is C2-C4 alkyl. In a further aspect, L is —(C1-C4 alkyl)(OCH₂CH₂). In a still further aspect, L is a structure selected from:

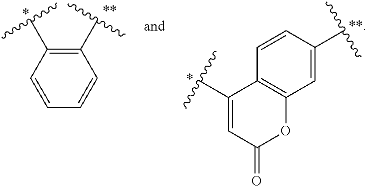 [SMILES: *c1ccccc1* ; *c1ccc2c(*)cc(=O)oc2c1]

In various aspects, R¹ is selected from —CO₂H and a structure:

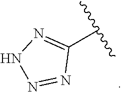 [SMILES: *c1nn[nH]n1]

In various aspects, R¹ is —CO₂H.

In various aspects, R² is a residue of a side chain of an amino acid selected from alanine, valine, leucine, serine, cysteine, methionine, arginine, lysine, asparagine, glycine, phenylalanine, tyrosine, and tryptophan. In a further aspect, R² is a residue of a side chain of an amino acid selected from alanine, leucine, serine, cysteine, methionine, arginine, lysine, asparagine, and glycine.

In various aspects, one of R^{3a} and R^{3b}, when present, is hydrogen, and one of R^{3a} and R^{3b}, when present, is a residue of a side chain of an amino acid selected from alanine, valine, leucine, serine, cysteine, methionine, arginine, lysine, asparagine, glycine, phenylalanine, tyrosine, and tryptophan.

In various aspects, R⁴ is hydrogen. In a further aspect, R⁴ is methyl.

In various aspects, each occurrence of R⁵, when present, is independently a residue of a side chain of an amino acid selected from alanine, valine, leucine, serine, cysteine, methionine, arginine, lysine, asparagine, glycine, phenylalanine, tyrosine, and tryptophan.

In various aspects, each occurrence of R⁶, when present, is hydrogen. In a further aspect, each occurrence of R⁶, when present, is methyl.

In various aspects, each of R^{7a} and R^{7b}, when present, is hydrogen. In a further aspect, each of R^{7a} and R^{7b}, when present, is methyl.

In various aspects, the compound has a structure represented by a formula:

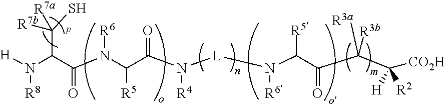

In various aspects, the compound has a structure represented by a formula:

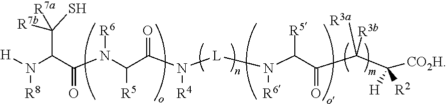

In various aspects, the compound has a structure represented by a formula:

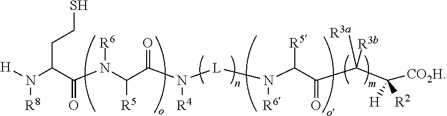

In various aspects, the compound has a structure represented by a formula:

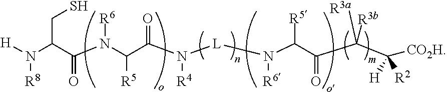

In various aspects, the compound has a structure represented by a formula:

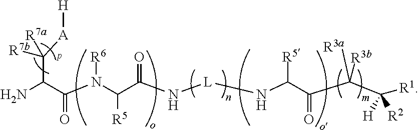

In various aspects, the compound has a structure represented by a formula:

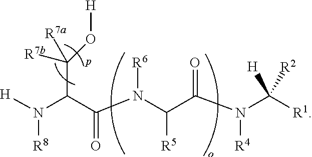

In various aspects, the compound has a structure represented by a formula:

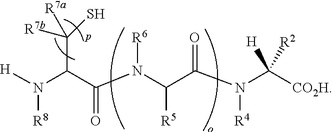

In various aspects, the compound has a structure represented by a formula:

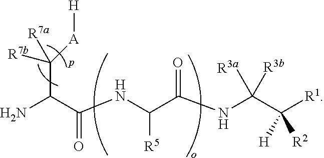

In a further aspect, o is 1, 2, 3, 4, 5, 6, 7, 8, or 9. In a still further aspect, one of R^{3a} and R^{3b} when present, is hydrogen, and one of R^{3a} and R^{3b}, when present, is a residue of a side chain of an amino acid selected from alanine, valine, leucine, serine, cysteine, methionine, arginine, lysine, asparagine, glycine, phenylalanine, tyrosine, and tryptophan.

In various aspects, the compound has a structure represented by a formula:

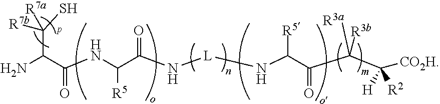

In various aspects, the compound has a structure represented by a formula:

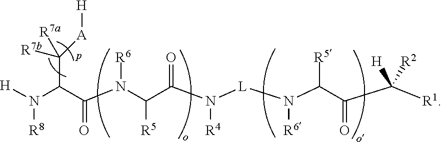

In various aspects, the compound has a structure represented by a formula:

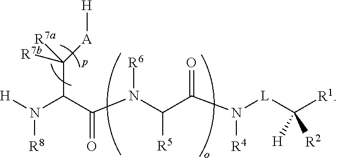

In various aspects, the compound has a structure represented by a formula:

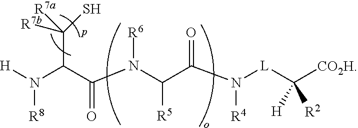

In a further aspect, o is 1, 2, 3, 4, 5, 6, 7, 8, or 9.

In various aspects, the compound has a structure represented by a formula:

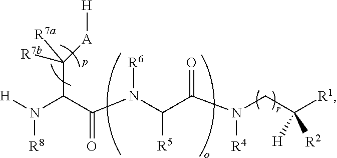

wherein r is 2, 3, or 4.

In various aspects, the compound has a structure represented by a formula:

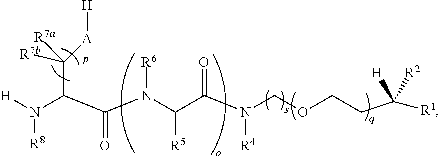

wherein s is 1 or 2.

In various aspects, the compound has a structure represented by a formula:

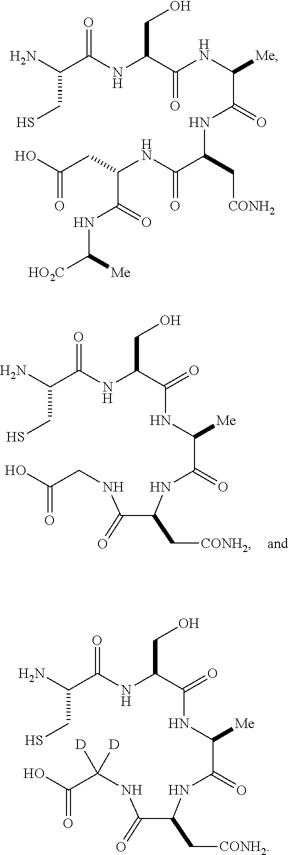 [SMILES: C[C@H](NC(=O)[C@H](CC(=O)O)NC(=O)[C@H](CC(N)=O)NC(=O)[C@H](C)NC(=O)[C@H](CO)NC(=O)[C@@H](N)CS)C(=O)O ; C[C@H](NC(=O)[C@H](CO)NC(=O)[C@@H](N)CS)C(=O)N[C@@H](CC(N)=O)C(=O)NCC(=O)O ; "and" ; [2H]C([2H])(NC(=O)[C@H](CC(N)=O)NC(=O)[C@H](C)NC(=O)[C@H](CO)NC(=O)[C@@H](N)CS)C(=O)O]

In various aspects, the thioether compound has a structure represented by a formula:

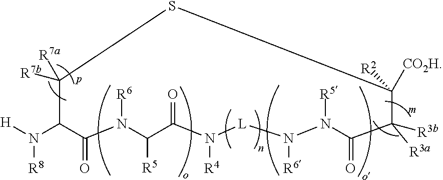

In various aspects, the thioether or selenoether compound has a structure represented by a formula:

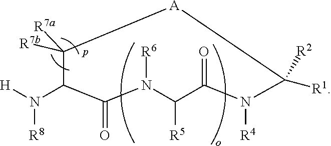

In various aspects, the thioether compound has a structure represented by a formula:

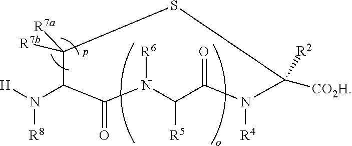

In various aspects, the thioether or selenoether compound has a structure represented by a formula:

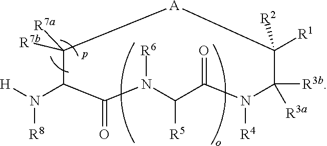

In various aspects, the thioether or selenoether compound has a structure represented by a formula:

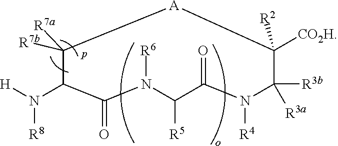

In various aspects, the thioether or selenoether compound has a structure represented by a formula:

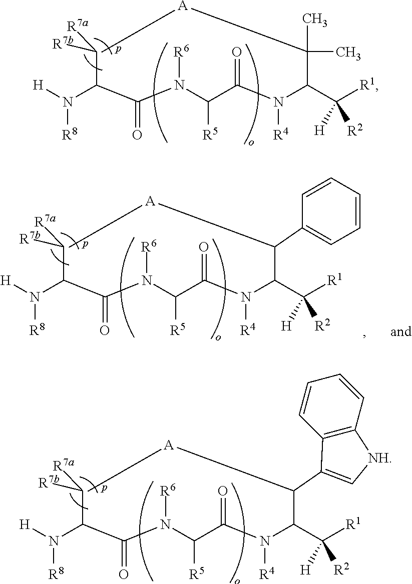

In various aspects, the thioether compound has a structure represented by a formula:

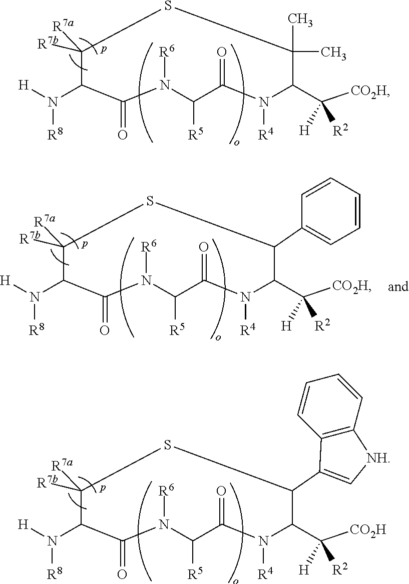

In various aspects, the thioether or selenoether compound has a structure represented by a formula:

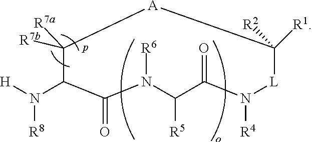

In various aspects, the thioether compound has a structure represented by a formula:

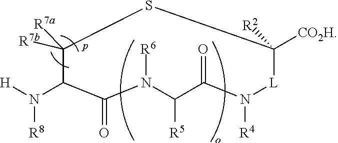

In various aspects, the thioether compound has a structure represented by a formula selected from:

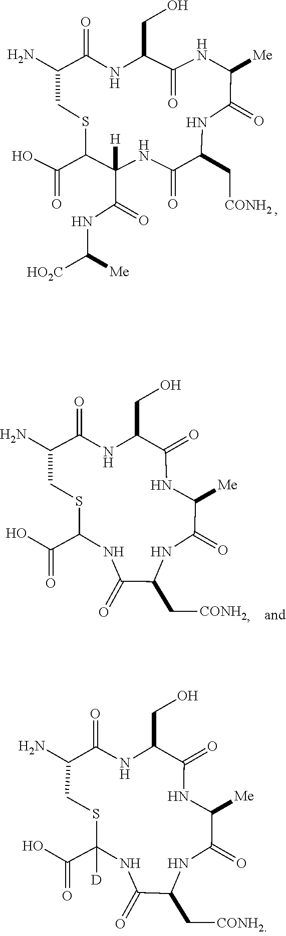 [SMILES: C[C@H](NC(=O)[C@H]1NC(=O)[C@H](CC(N)=O)NC(=O)[C@H](C)NC(=O)[C@H](CO)NC(=O)[C@@H](N)CSC1C(=O)O)C(=O)O ; C[C@@H]1NC(=O)[C@H](CO)NC(=O)[C@@H](N)CSC(C(=O)O)NC(=O)[C@H](CC(N)=O)NC1=O ; [2H]C1(C(=O)O)NC(=O)[C@H](CC(N)=O)NC(=O)[C@H](C)NC(=O)[C@H](CO)NC(=O)[C@@H](N)CS1]

In various aspects, the thioether compound has a structure represented by a formula selected from:

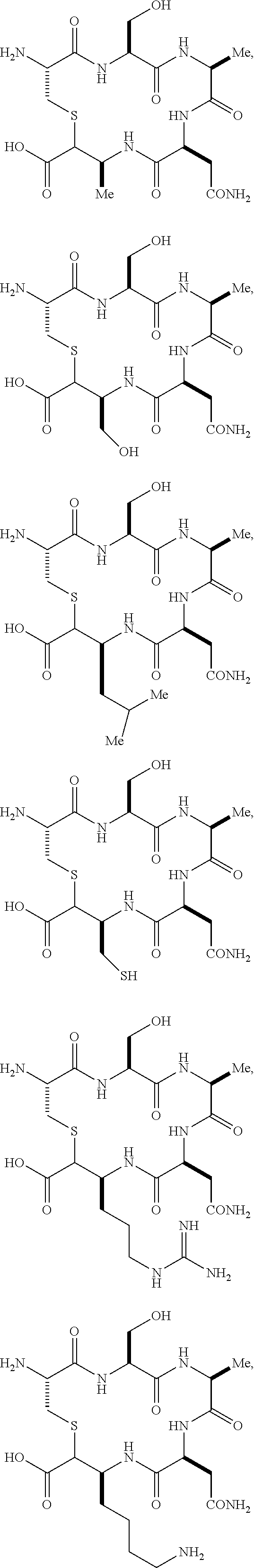

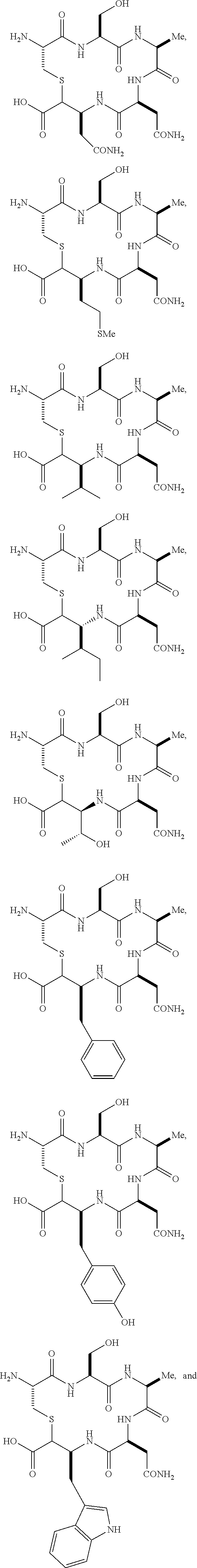

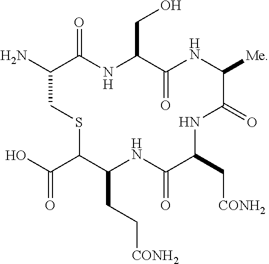 [SMILES: C[C@@H]1NC(=O)[C@H](CO)NC(=O)[C@@H](N)CSC(C(=O)O)[C@H](CCC(N)=O)NC(=O)[C@H](CC(N)=O)NC1=O]

In various aspects, the thioether compound has a structure represented by a formula selected from:

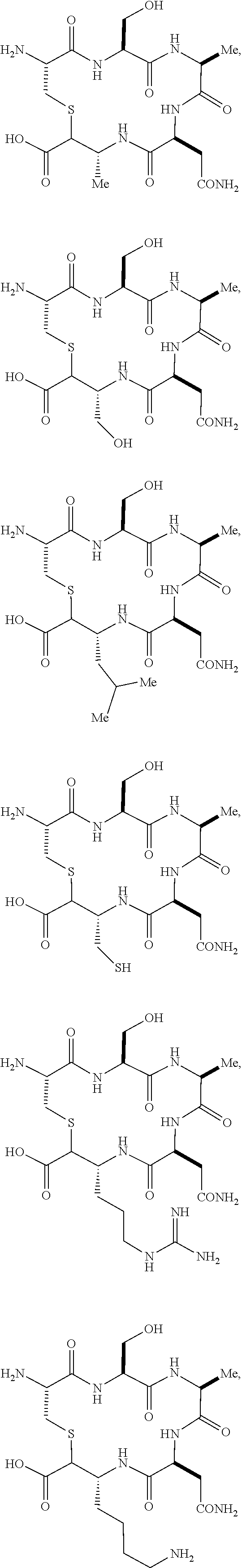

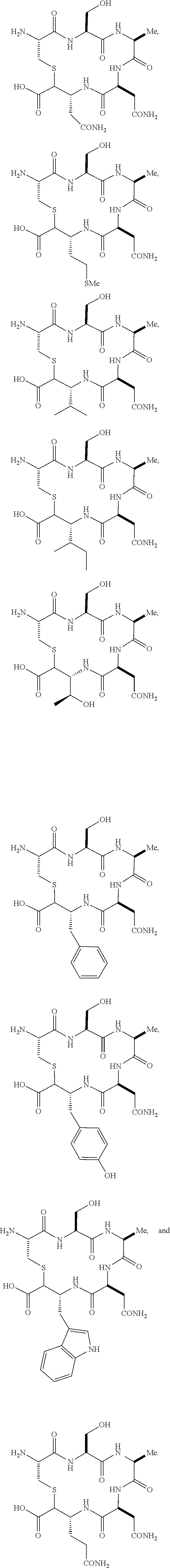

In various aspects, the thioether compound has a structure represented by a formula selected from:

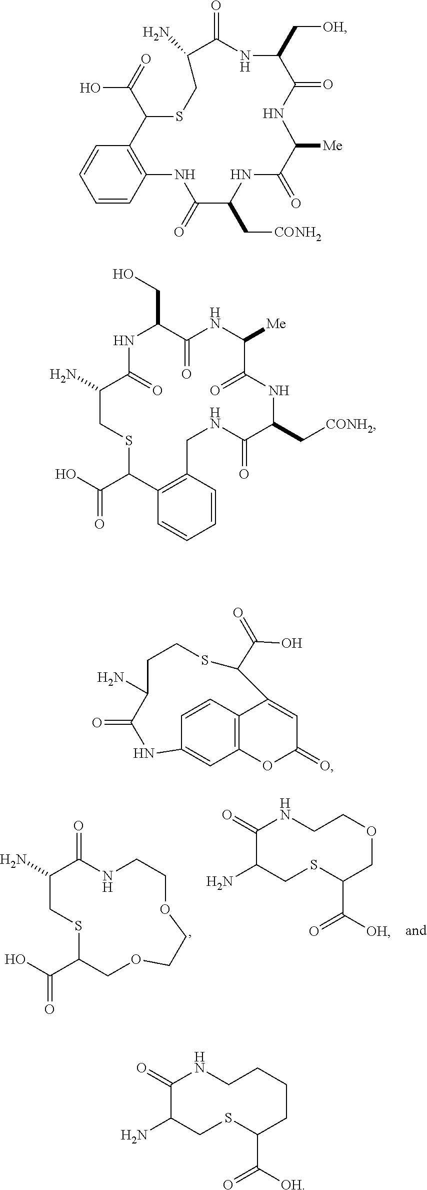 [SMILES: C[C@@H]1NC(=O)[C@H](CO)NC(=O)[C@@H](N)CSC(C(=O)O)c2ccccc2NC(=O)[C@H](CC(N)=O)NC1=O ; C[C@@H]1NC(=O)[C@H](CO)NC(=O)[C@@H](N)CSC(C(=O)O)c2ccccc2CNC(=O)[C@H](CC(N)=O)NC1=O ; NC1CCSC(C(=O)O)c2cc(=O)oc3cc(ccc23)NC1=O ; N[C@H]1CSC(C(=O)O)COCCOCCNC1=O ; NC1CSC(C(=O)O)COCCNC1=O ; NC1CSC(C(=O)O)CCCCNC1=O]

In various aspects, the thioether compound is a sactipeptide. In a further aspect, the sactipeptide has a structure represented by a formula selected from:

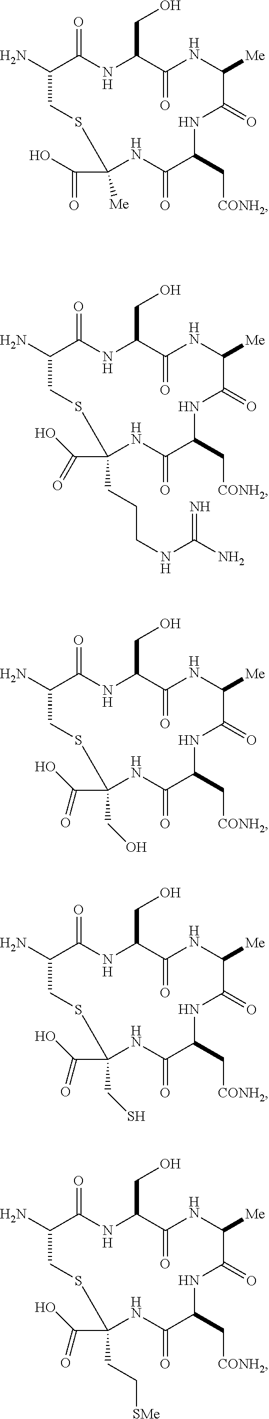 [SMILES: C[C@@H]1NC(=O)[C@H](CO)NC(=O)[C@@H](N)CS[C@](C)(C(=O)O)NC(=O)[C@H](CC(N)=O)NC1=O ; C[C@@H]1NC(=O)[C@H](CO)NC(=O)[C@@H](N)CS[C@](CCCNC(=N)N)(C(=O)O)NC(=O)[C@H](CC(N)=O)NC1=O ; C[C@@H]1NC(=O)[C@H](CO)NC(=O)[C@@H](N)CS[C@](CO)(C(=O)O)NC(=O)[C@H](CC(N)=O)NC1=O ; C[C@@H]1NC(=O)[C@H](CO)NC(=O)[C@@H](N)CS[C@](CS)(C(=O)O)NC(=O)[C@H](CC(N)=O)NC1=O ; CSCC[C@@]1(C(=O)O)NC(=O)[C@H](CC(N)=O)NC(=O)[C@H](C)NC(=O)[C@H](CO)NC(=O)[C@@H](N)CS1]

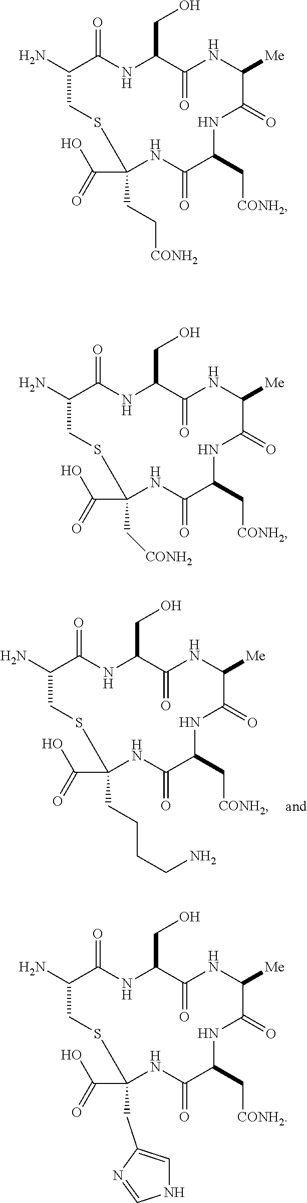 [SMILES: C[C@@H]1NC(=O)[C@H](CO)NC(=O)[C@@H](N)CS[C@](CCC(N)=O)(C(=O)O)NC(=O)[C@H](CC(N)=O)NC1=O ; C[C@@H]1NC(=O)[C@H](CO)NC(=O)[C@@H](N)CS[C@](CC(N)=O)(C(=O)O)NC(=O)[C@H](CC(N)=O)NC1=O ; C[C@@H]1NC(=O)[C@H](CO)NC(=O)[C@@H](N)CS[C@](CCCCN)(C(=O)O)NC(=O)[C@H](CC(N)=O)NC1=O ; "and" ; C[C@@H]1NC(=O)[C@H](CO)NC(=O)[C@@H](N)CS[C@](Cc2c[nH]cn2)(C(=O)O)NC(=O)[C@H](CC(N)=O)NC1=O]

In various aspects, the thioether compound is a ranthipeptide. In a further aspect, the ranthipeptide has a structure represented by a formula selected from:

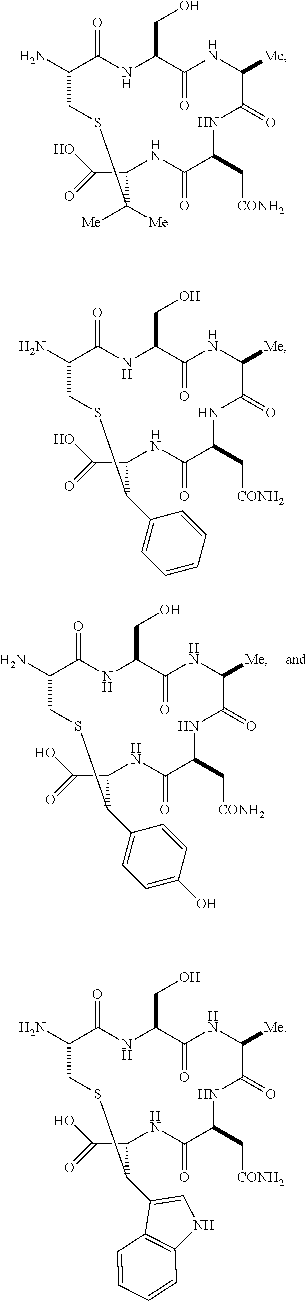

In various aspects, the one or more of the compounds or thioether compounds disclosed herein is fused to a host cell surface protein.

In various aspects, the host cell is a bacterial cell.

In various aspects, the host cell is a yeast cell.

In various aspects, the host cell surface protein is a yeast anchor protein.

In various aspects, the host cell surface protein is an outer membrane protein.

In various aspects, the cell-surface display library further comprising a nucleic acid sequence capable of encoding a secretion signal.

### F. METHODS FOR MAKING A YEAST CELL SURFACE DISPLAY

In one aspect, disclosed are methods for making a yeast cell surface display, the method comprising transforming yeast cells with a vector comprising a nucleic acid sequence capable of encoding one or more of the compounds disclosed herein fused to a yeast cell wall protein.

In various aspects, the compound has a structure represented by a formula:

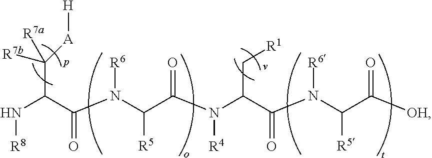

wherein o is 0, 1, 2, 3, 4, 5, 6, 7, 8, or 9; wherein p is 1 or 2; wherein t is an integer from 0 to 500; wherein v is 1, 2, 3, 4, or 5; wherein A is S or Se; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

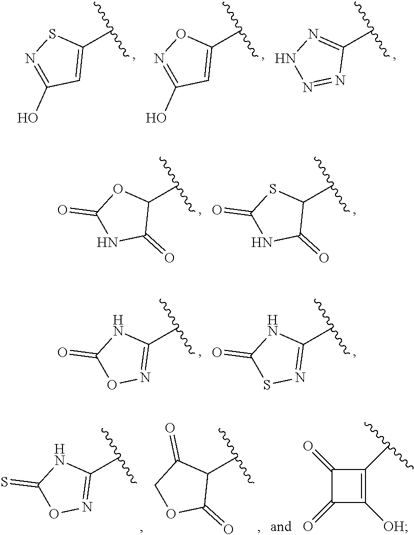

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, provided that the compound is not PapA.

In various aspects, the compound has a structure represented by a formula:

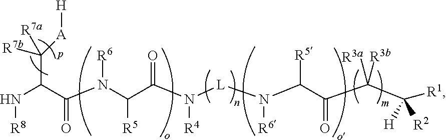

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein A is S or Se; wherein L, when present, is selected from C2-C4 alkyl, —(C1-C4 alkyl)(OCH₂CH₂)_{q}, and a structure selected from:

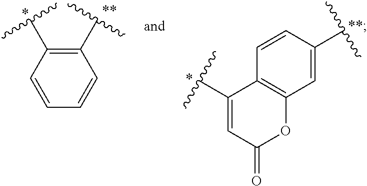

wherein q is 1, 2, 3, or 4; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

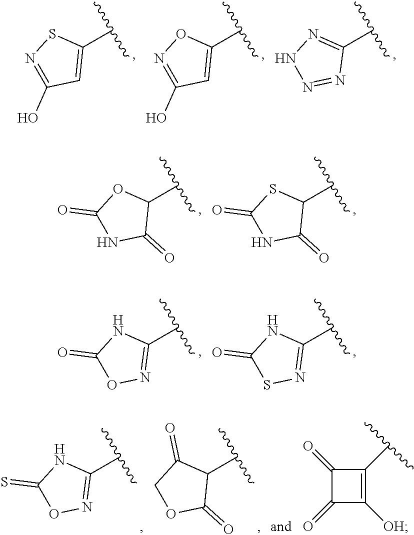

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, provided that the compound is not PapA.

In various aspects, the thioether or selenoether compound has a structure represented by a formula:

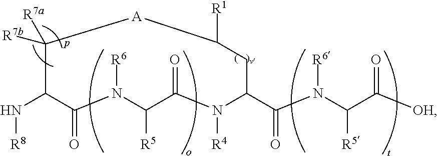

wherein o is 0, 1, 2, 3, 4, 5, 6, 7, 8, or 9; wherein p is 1 or 2; wherein t is an integer from 0 to 500; wherein v′ is 0, 1, 2, 3, or 4; wherein A is S or Se; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

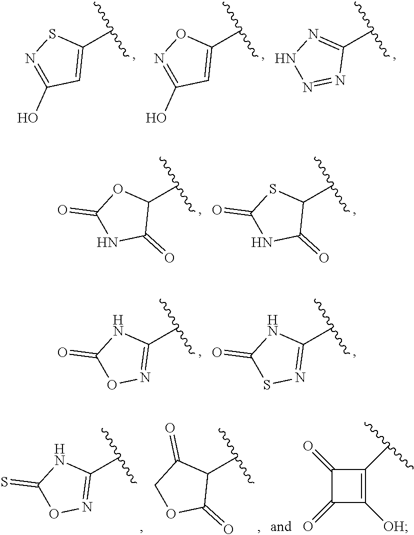

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl.

In various aspects, the thioether or selenoether compound has a structure represented by a formula:

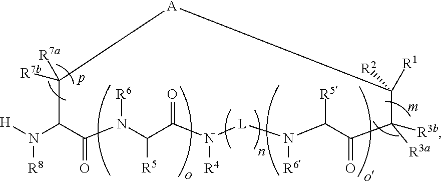

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein A is S or Se; wherein L, when present, is selected from C2-C4 alkyl, —(C1-C4 alkyl)(OCH₂CH₂)_{q}, and a structure selected from:

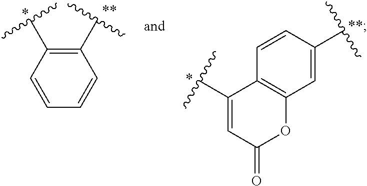

wherein q is 1, 2, 3, or 4; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

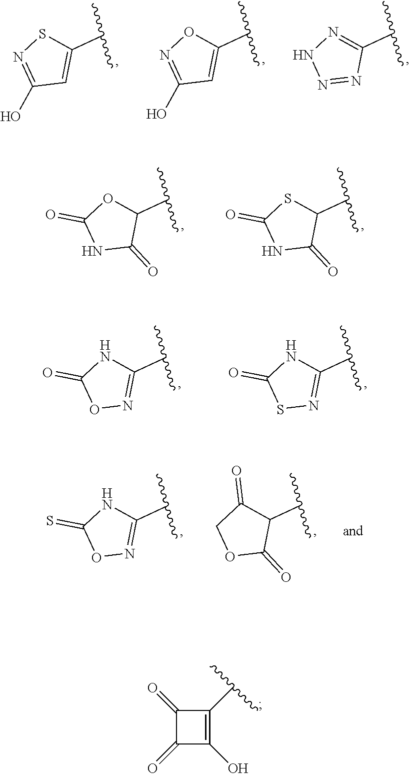

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl.

In various aspects, o is independently 0, 1, 2, 3, 4, 5, 6, or 7.

In various aspects, t is 0.

In various aspects, v is 1 or 2.

In various aspects, w is 0 or 1.

In various aspects, R¹ is —CO₂H or a structure:

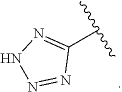 [SMILES: *c1nn[nH]n1]

In various aspects, R¹ is —CO₂H.

In various aspects, the compound comprises one or more D-amino acid residues.

In various aspects, the compound comprises one or more p-amino acid residues.

In various aspects, the compound comprises one or more N-methylated amino acid residues.

In various aspects, the compound has a structure represented by a formula:

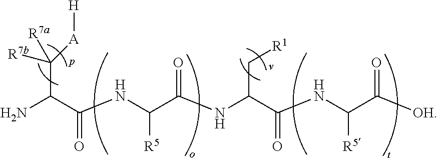

In various aspects, the compound has a structure represented by a formula:

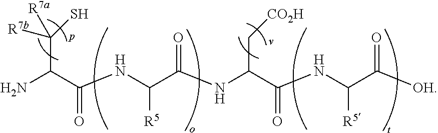

In various aspects, the method produces a thioether or selenoether compound has a structure represented by a formula:

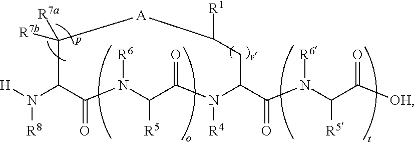

wherein v′ is 0, 1, 2, or 3.

In various aspects, m is 0. In a further aspect, m is 1.

In various aspects, n is 0. In a further aspect, n is 1.

In various aspects, o is 0, 1, 2, 3, 4, 5, 6, or 7. In a further aspect, o is 1, 2, 3, 4, 5, 6, 7, 8, or 9. In a still further aspect, o is 1, 2, 3, or 4.

In various aspects, p is 1. In a further aspect, p is 2.

In various aspects, w is 0. In a further aspect, w is 1.

In various aspects, A is S. In a further aspect, A is Se.

In various aspects, L is C2-C4 alkyl. In a further aspect, L is —(C1-C4 alkyl)(OCH₂CH₂)_{q}. In a still further aspect, L is a structure selected from:

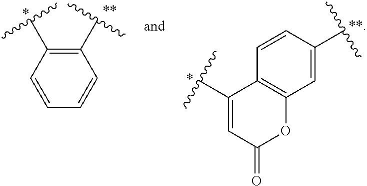 [SMILES: *c1ccccc1* ; *c1ccc2c(*)cc(=O)oc2c1]

In various aspects, R¹ is selected from —CO₂H and a structure:

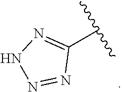 [SMILES: *c1nn[nH]n1]

In various aspects, R¹ is —CO₂H.

In various aspects, R² is a residue of a side chain of an amino acid selected from alanine, valine, leucine, serine, cysteine, methionine, arginine, lysine, asparagine, glycine, phenylalanine, tyrosine, and tryptophan. In a further aspect, R² is a residue of a side chain of an amino acid selected from alanine, leucine, serine, cysteine, methionine, arginine, lysine, asparagine, and glycine.

In various aspects, one of R^{3a} and R^{3b}, when present, is hydrogen, and one of R^{3a} and R^{3b}, when present, is a residue of a side chain of an amino acid selected from alanine, valine, leucine, serine, cysteine, methionine, arginine, lysine, asparagine, glycine, phenylalanine, tyrosine, and tryptophan.

In various aspects, R⁴ is hydrogen. In a further aspect, R⁴ is methyl.

In various aspects, each occurrence of R⁵, when present, is independently a residue of a side chain of an amino acid selected from alanine, valine, leucine, serine, cysteine, methionine, arginine, lysine, asparagine, glycine, phenylalanine, tyrosine, and tryptophan.

In various aspects, each occurrence of R⁶, when present, is hydrogen. In a further aspect, each occurrence of R⁶, when present, is methyl.

In various aspects, each of R^{7a} and R^{7b}, when present, is hydrogen. In a further aspect, each of R^{7a} and R^{7b}, when present, is methyl.

In various aspects, the compound has a structure represented by a formula:

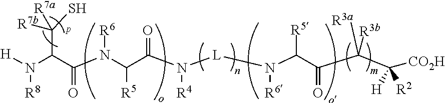

In various aspects, the compound has a structure represented by a formula:

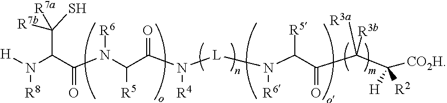

In various aspects, the compound has a structure represented by a formula:

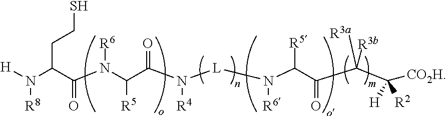

In various aspects, the compound has a structure represented by a formula:

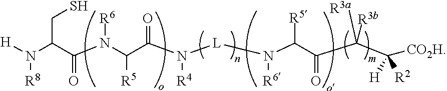

In various aspects, the compound has a structure represented by a formula:

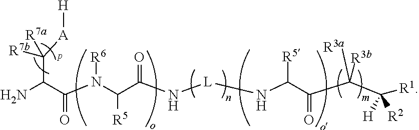

In various aspects, the compound has a structure represented by a formula:

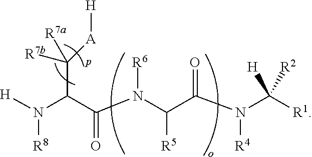

In various aspects, the compound has a structure represented by a formula:

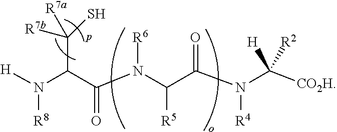

In various aspects, the compound has a structure represented by a formula:

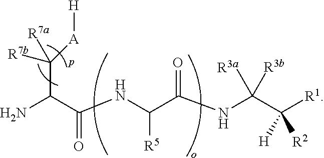

In a further aspect, o is 1, 2, 3, 4, 5, 6, 7, 8, or 9. In a still further aspect, one of R^{3a} and R^{3b}, when present, is hydrogen, and one of R^{3a} and R^{3b}, when present, is a residue of a side chain of an amino acid selected from alanine, valine, leucine, serine, cysteine, methionine, arginine, lysine, asparagine, glycine, phenylalanine, tyrosine, and tryptophan.

In various aspects, the compound has a structure represented by a formula:

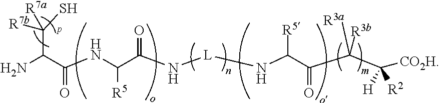

In various aspects, the compound has a structure represented by a formula:

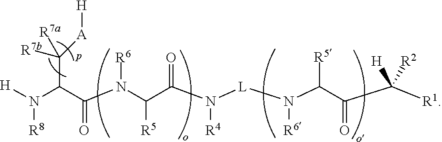

In various aspects, the compound has a structure represented by a formula:

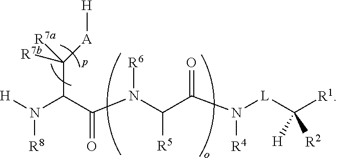

In various aspects, the compound has a structure represented by a formula:

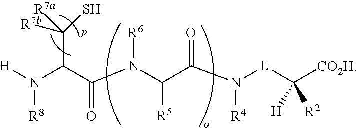

In a further aspect, o is 1, 2, 3, 4, 5, 6, 7, 8, or 9.

In various aspects, the compound has a structure represented by a formula:

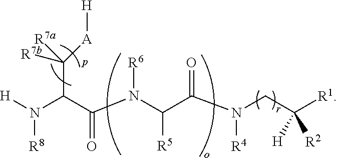

wherein r is 2, 3, or 4.

In various aspects, the compound has a structure represented by a formula:

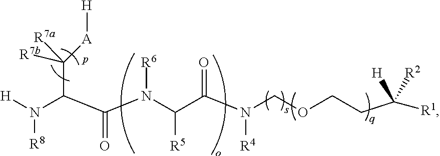

wherein s is 1 or 2.

In various aspects, the compound has a structure represented by a formula:

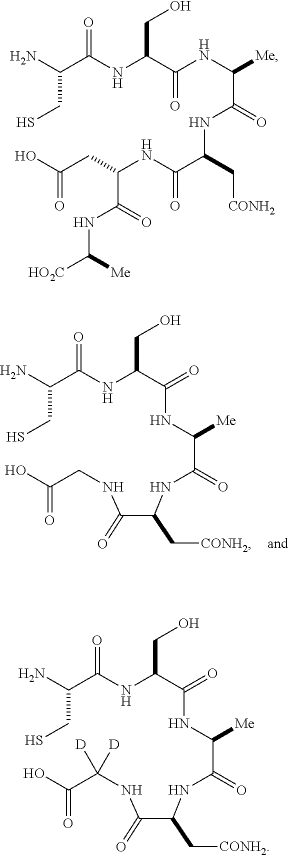 [SMILES: C[C@H](NC(=O)[C@H](CC(=O)O)NC(=O)[C@H](CC(N)=O)NC(=O)[C@H](C)NC(=O)[C@H](CO)NC(=O)[C@@H](N)CS)C(=O)O ; C[C@H](NC(=O)[C@H](CO)NC(=O)[C@@H](N)CS)C(=O)N[C@@H](CC(N)=O)C(=O)NCC(=O)O ; "and" ; [2H]C([2H])(NC(=O)[C@H](CC(N)=O)NC(=O)[C@H](C)NC(=O)[C@H](CO)NC(=O)[C@@H](N)CS)C(=O)O]

In various aspects, the thioether compound has a structure represented by a formula:

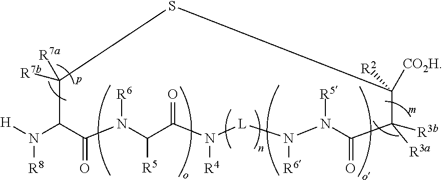

In various aspects, the thioether or selenoether compound has a structure represented by a formula:

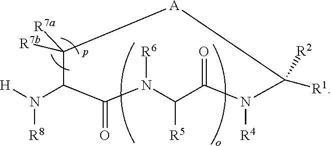

In various aspects, the thioether compound has a structure represented by a formula:

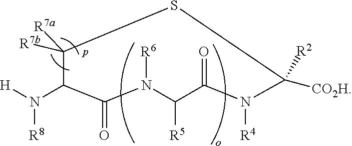

In various aspects, the thioether or selenoether compound has a structure represented by a formula:

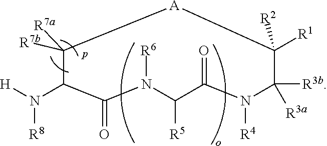

In various aspects, the thioether or selenoether compound has a structure represented by a formula:

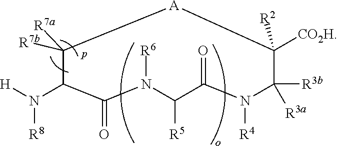

In various aspects, the thioether or selenoether compound has a structure represented by a formula:

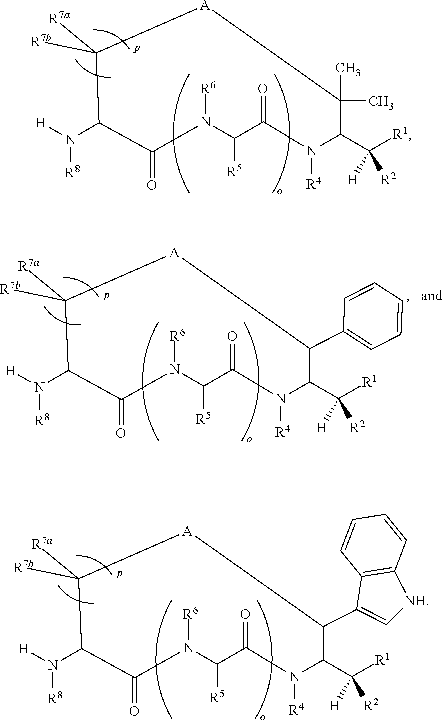

In various aspects, the thioether compound has a structure represented by a formula:

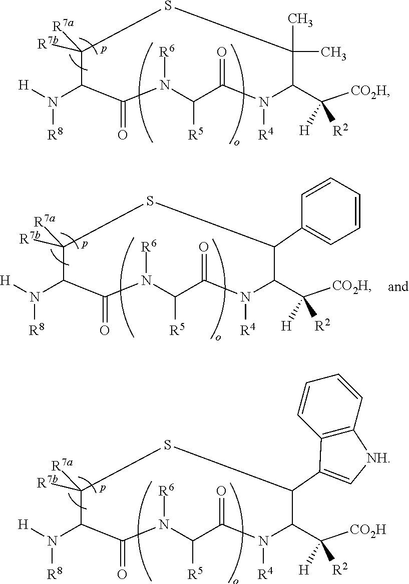

In various aspects, the thioether or selenoether compound has a structure represented by a formula:

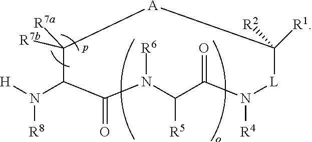

In various aspects, the thioether compound has a structure represented by a formula:

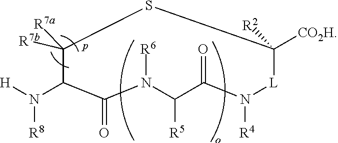

In various aspects, the thioether compound has a structure represented by a formula selected from:

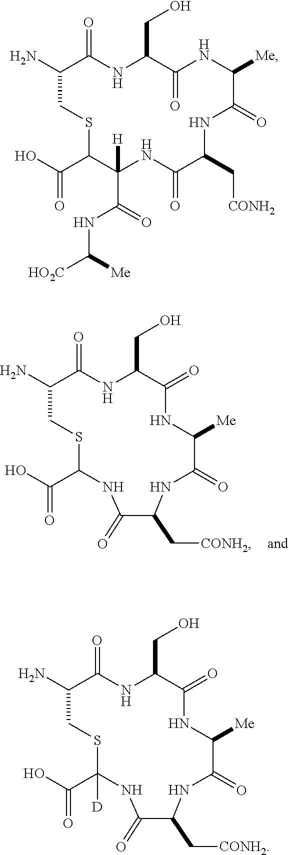 [SMILES: C[C@H](NC(=O)[C@H]1NC(=O)[C@H](CC(N)=O)NC(=O)[C@H](C)NC(=O)[C@H](CO)NC(=O)[C@@H](N)CSC1C(=O)O)C(=O)O ; C[C@@H]1NC(=O)[C@H](CO)NC(=O)[C@@H](N)CSC(C(=O)O)NC(=O)[C@H](CC(N)=O)NC1=O ; "and" ; [2H]C1(C(=O)O)NC(=O)[C@H](CC(N)=O)NC(=O)[C@H](C)NC(=O)[C@H](CO)NC(=O)[C@@H](N)CS1]

In various aspects, the thioether compound has a structure represented by a formula selected from:

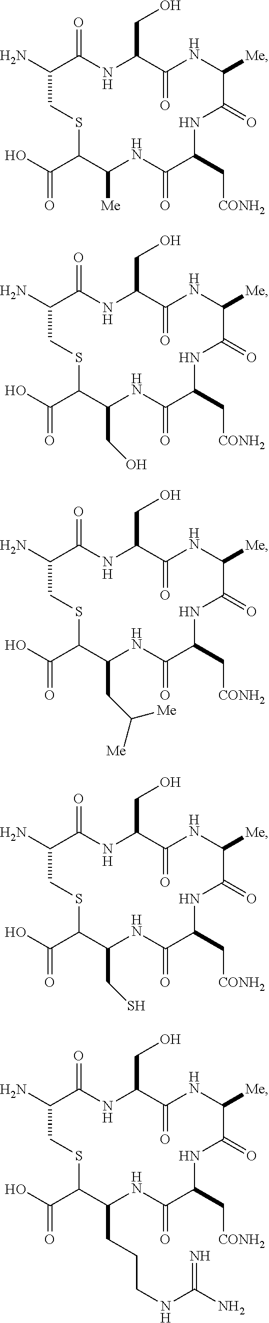

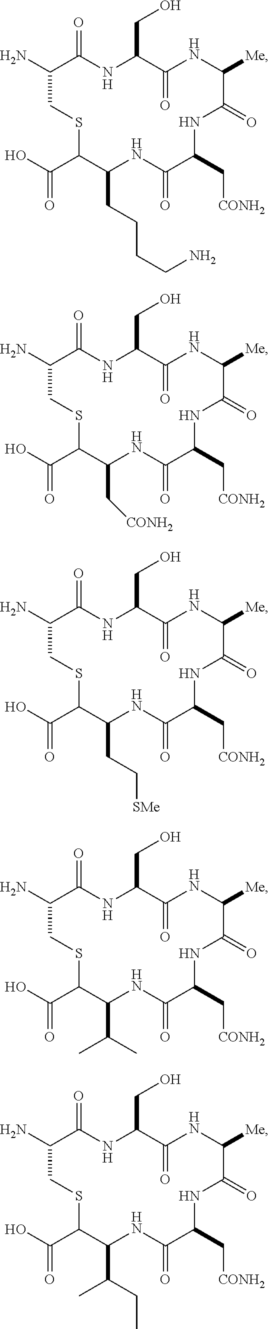

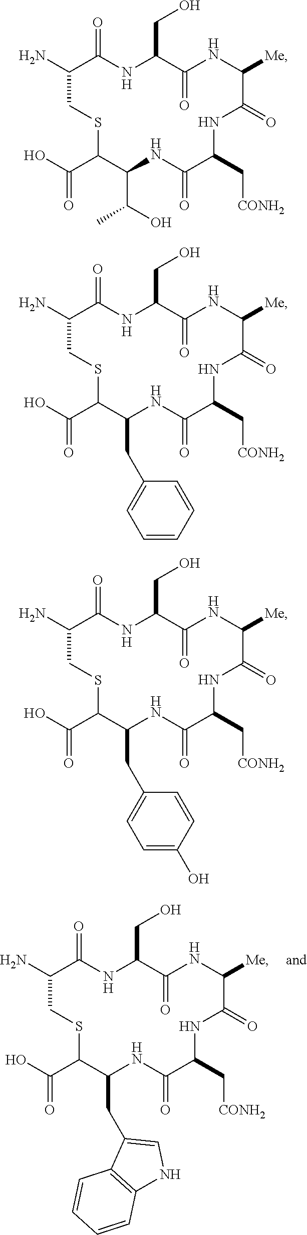

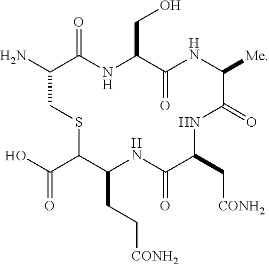 [SMILES: C[C@@H]1NC(=O)[C@H](CO)NC(=O)[C@@H](N)CSC(C(=O)O)[C@H](CCC(N)=O)NC(=O)[C@H](CC(N)=O)NC1=O]

In various aspects, the thioether compound has a structure represented by a formula selected from:

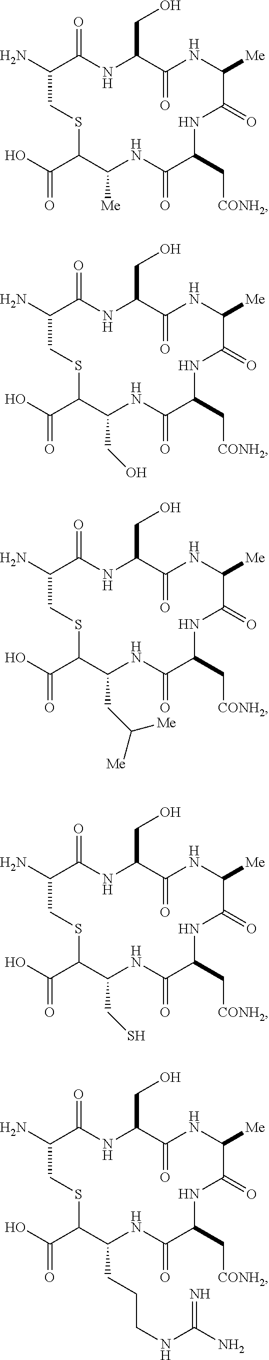

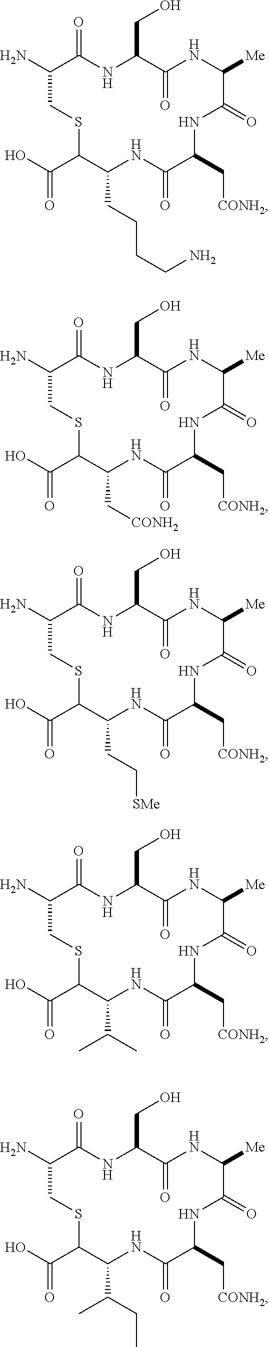

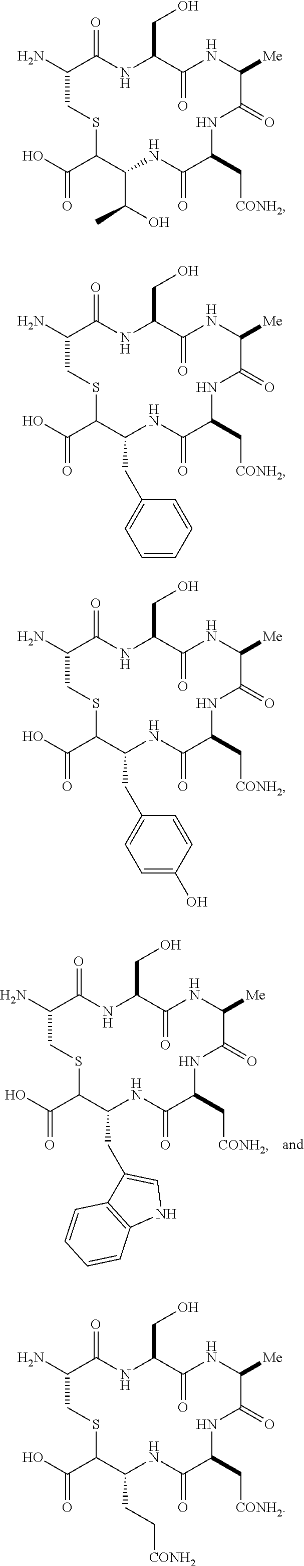

In various aspects, the thioether compound has a structure represented by a formula selected from:

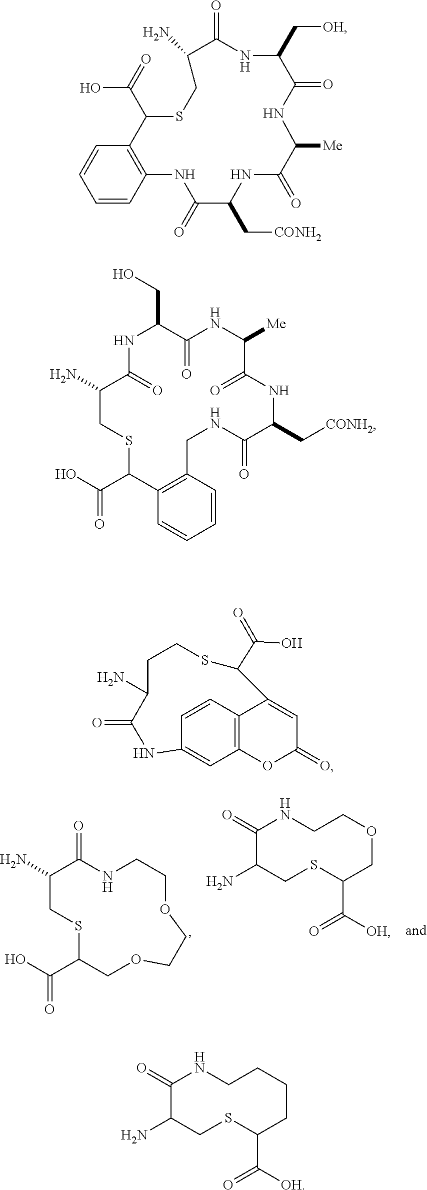 [SMILES: C[C@@H]1NC(=O)[C@H](CO)NC(=O)[C@@H](N)CSC(C(=O)O)c2ccccc2NC(=O)[C@H](CC(N)=O)NC1=O ; C[C@@H]1NC(=O)[C@H](CO)NC(=O)[C@@H](N)CSC(C(=O)O)c2ccccc2CNC(=O)[C@H](CC(N)=O)NC1=O ; NC1CCSC(C(=O)O)c2cc(=O)oc3cc(ccc23)NC1=O ; N[C@H]1CSC(C(=O)O)COCCOCCNC1=O ; NC1CSC(C(=O)O)COCCNC1=O ; NC1CSC(C(=O)O)CCCCNC1=O]

In various aspects, the thioether compound is a sactipeptide. In a further aspect, the sactipeptide has a structure represented by a formula selected from:

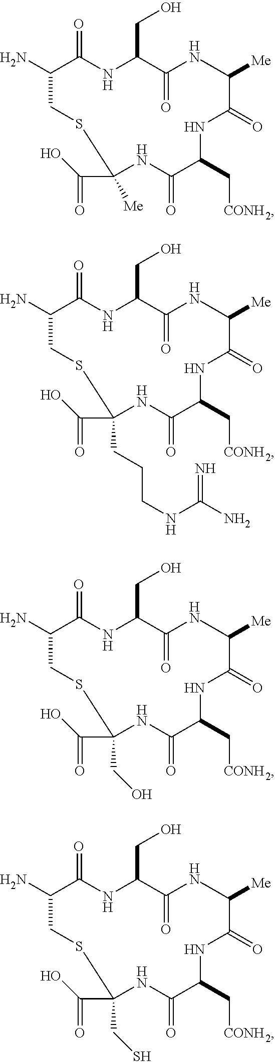

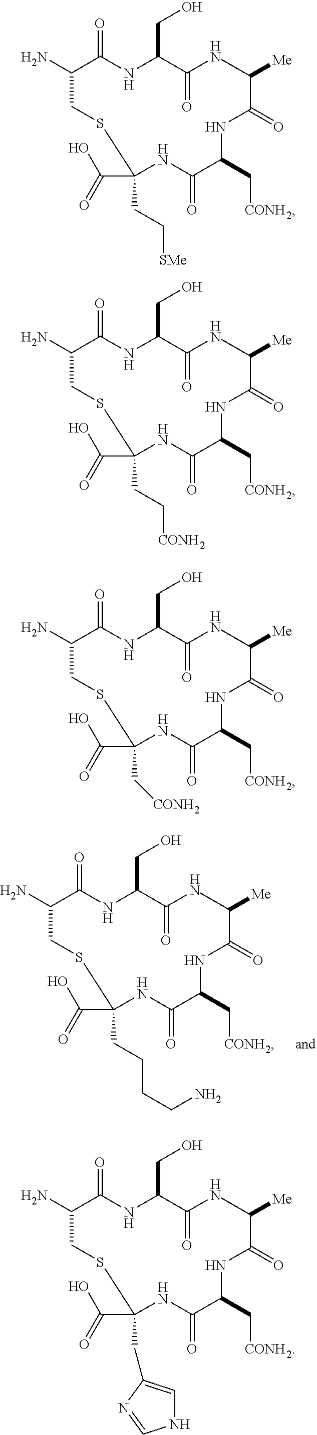

In various aspects, the thioether compound is a ranthipeptide. In a further aspect, the ranthipeptide has a structure represented by a formula selected from:

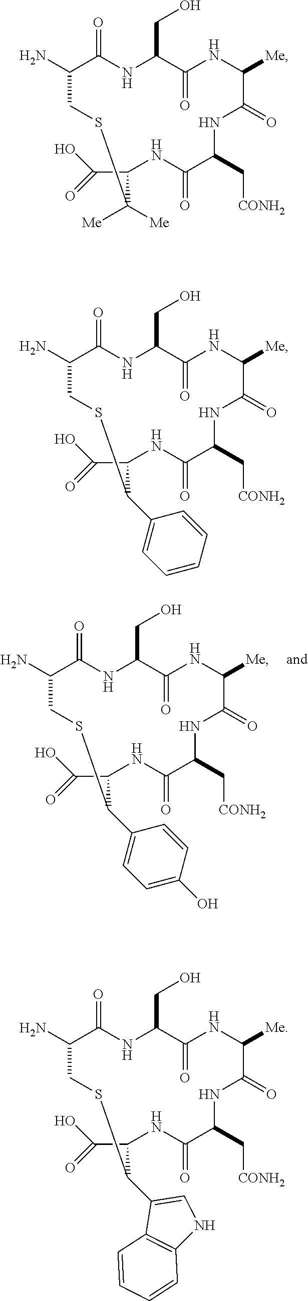

### G. ANALOGS OF PEPTIDE THERAPEUTICS

In one aspect, disclosed are thioether and selenoether compounds prepared from vectors or molecular displays, such as, for example, phage, ribosome, and cell-surface display libraries, wherein the thioether or selenoether compound is an analog of a peptide therapeutic. Exemplary peptide therapeutics include, but are not limited to, octreotide, setmalanotide, romidepsin, bremelanotide, pramlintide, oxytocin, setmelanotide, or cyclosporin.

Thus, in one aspect, disclosed are thioether compounds having a structure selected from:

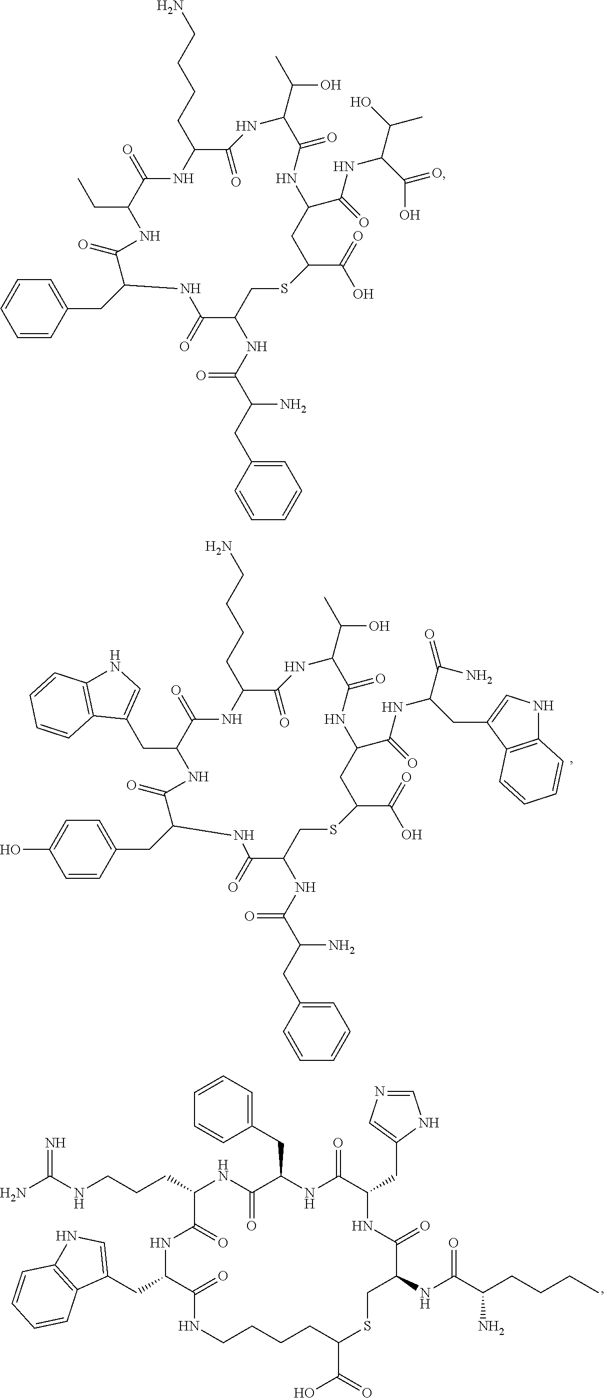

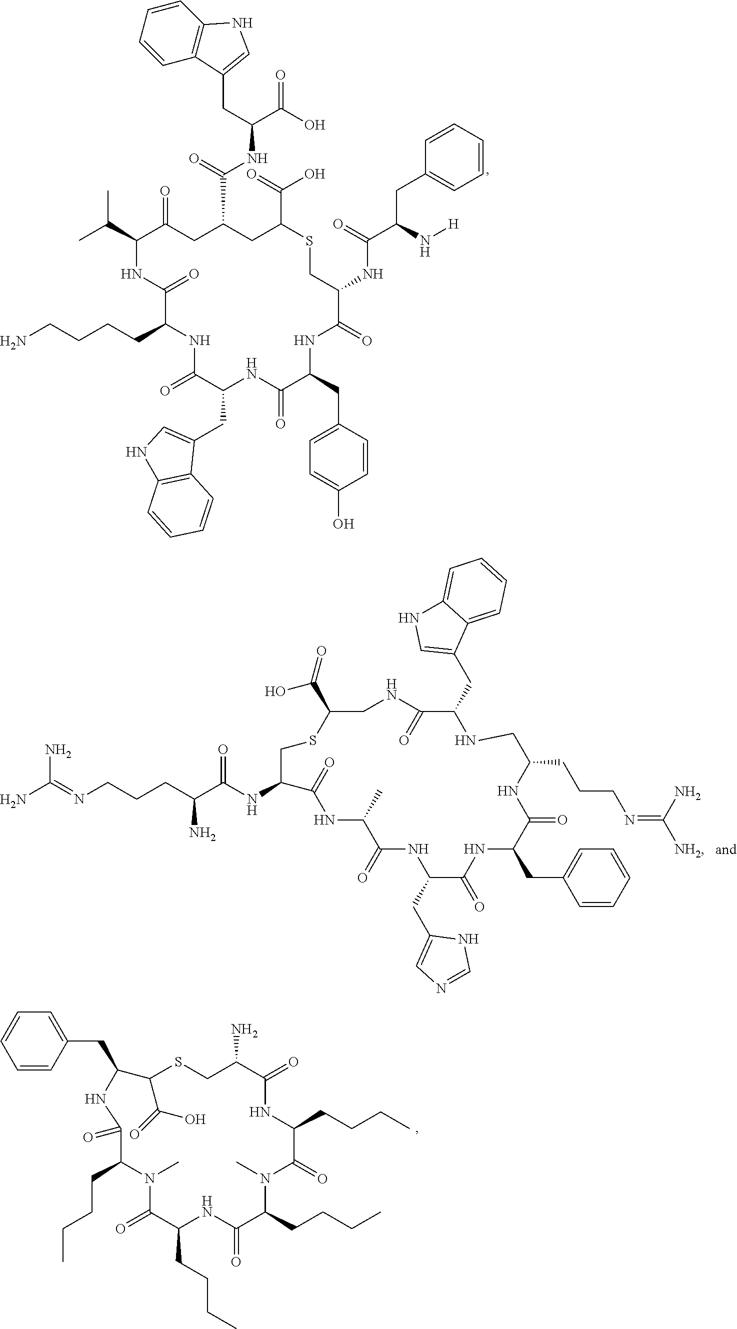 [SMILES: CC(C)[C@@H]1NC(=O)[C@H](CCCCN)NC(=O)[C@@H](Cc2c[nH]c3ccccc23)NC(=O)[C@H](Cc2ccc(O)cc2)NC(=O)[C@@H](NC(=O)[C@H](N)Cc2ccccc2)CSC(C(=O)O)C[C@@H](C(=O)N[C@@H](Cc2c[nH]c3ccccc23)C(=O)O)CC1=O ; C[C@H]1NC(=O)[C@@H](NC(=O)[C@@H](N)CCCN=C(N)N)CS[C@@H](C(=O)O)CNC(=O)[C@H](Cc2c[nH]c3ccccc23)NC[C@H](CCCN=C(N)N)NC(=O)[C@@H](Cc2ccccc2)NC(=O)[C@H](Cc2cnc[nH]2)NC1=O ; CCCC[C@@H]1NC(=O)[C@@H](N)CSC(C(=O)O)[C@H](Cc2ccccc2)NC(=O)[C@H](CCCC)N(C)C(=O)[C@H](CCCC)NC(=O)[C@H](CCCC)N(C)C1=O]

or a pharmaceutically acceptable salt thereof.

Also disclosed are thioether compounds selected from:

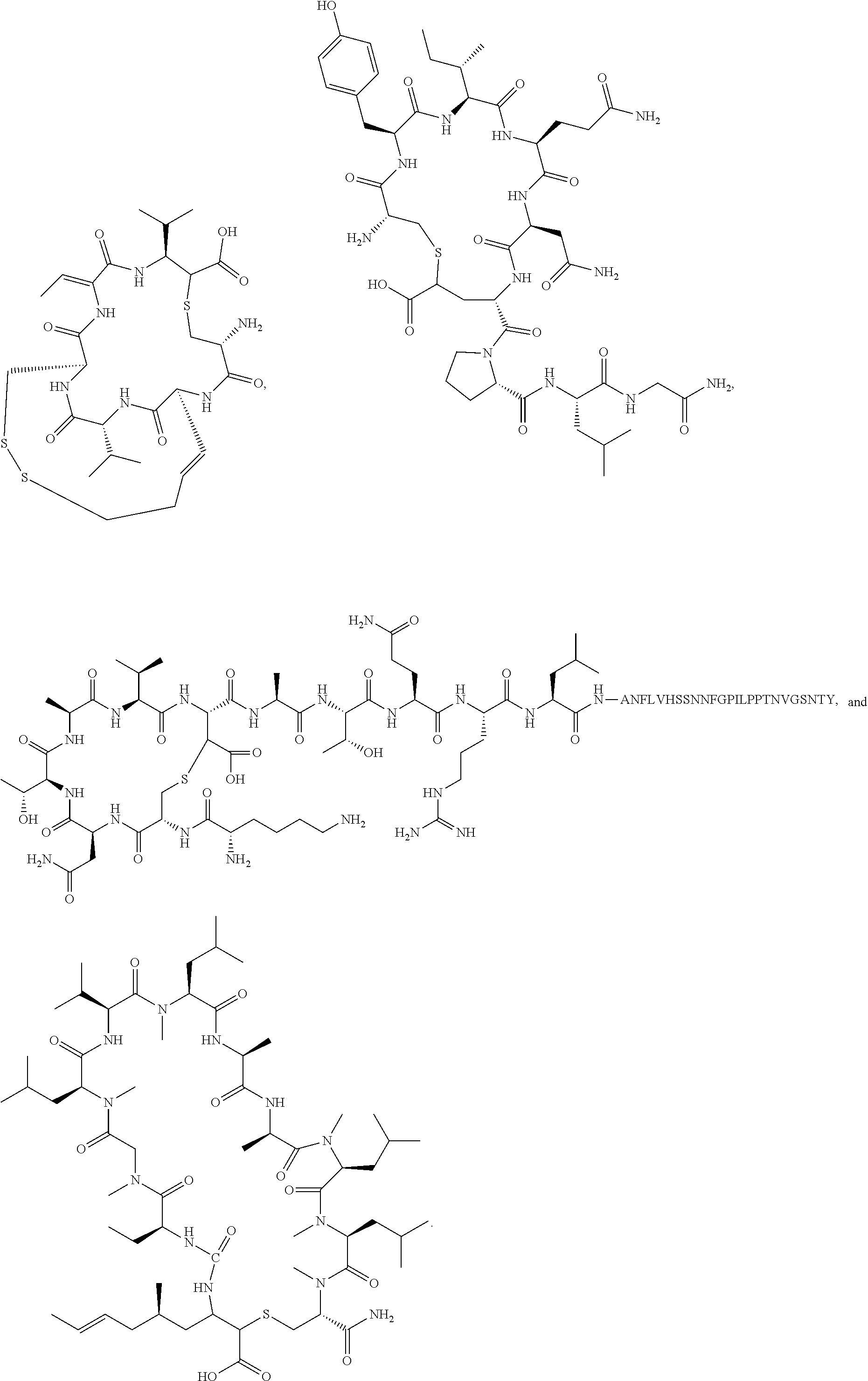

or a pharmaceutically acceptable salt thereof.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A vector comprising a nucleic acid sequence capable of encoding a compound having a structure represented by a formula selected from: 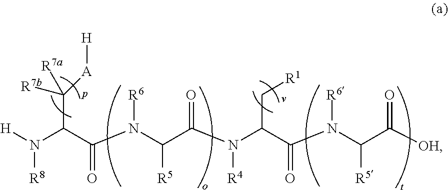
wherein o is 0, 1, 2, 3, 4, 5, 6, 7, 8, or 9;
wherein p is 1 or 2;
wherein t is an integer from 0 to 500;
wherein v is 1, 2, 3, 4, or 5;
wherein A is S or Se;
wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from: 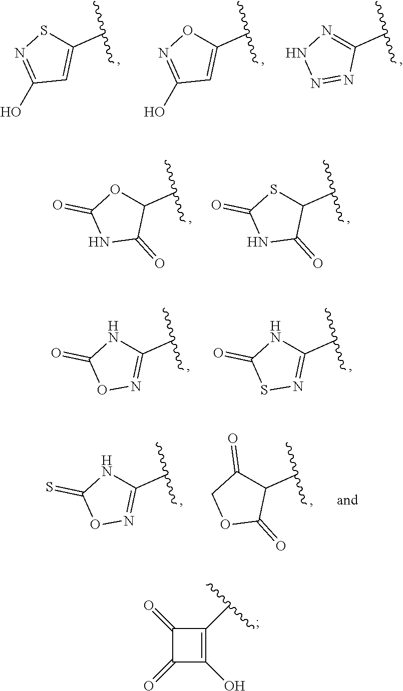
wherein R⁴ is selected from hydrogen and methyl;
wherein each occurrence of R⁵ and R^{5′}, when present, is independently a residue of a side chain of amino acid;
wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle;
wherein each of R^{7a} and R^{7b}, when present, is independently selected from hydrogen and C1-C4 alkyl; and
wherein R⁸ is selected from hydrogen and methyl; and 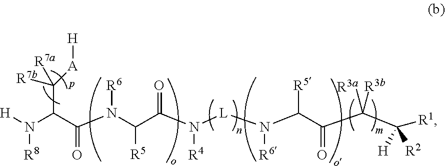
wherein m is 0, 1, 2, 3, or 4;
wherein n is 0 or 1;
wherein each of o and o′ is independently 0, 1, 2, 3, 4, 5, 6, 7, 8, or 9;
wherein p is 1 or 2;
wherein A is S or Se;
wherein L, when present, is selected from C2-C4 alkyl, —(C1-C4 alkyl)(OCH₂CH₂)_{q}, and a structure selected from: 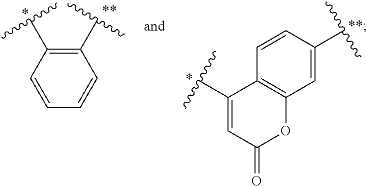
wherein q is 1, 2, 3, or 4;
wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from: 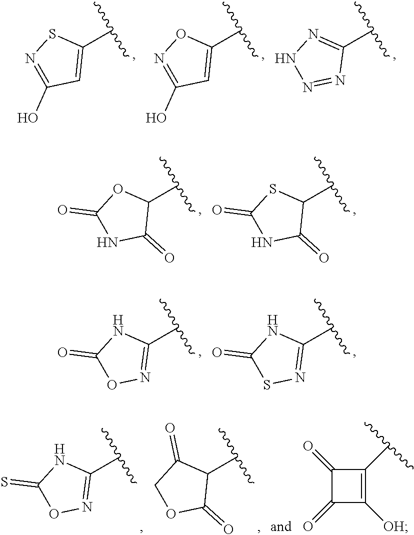
wherein R² is a residue of a side chain of amino acid, provided that the amino acid is not isoleucine or threonine;
wherein each of R^{3a} and R^{3b}, when present, is independently selected from C2-C5 alkynyl, C1-C5 azido, and a residue of a side chain of an amino acid;
wherein R⁴ is selected from hydrogen and methyl;
wherein each occurrence of R⁵ and R^{5′}, when present, is independently a residue of a side chain of amino acid;
wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle;
wherein each of R^{7a} and R⁷, when present, is independently selected from hydrogen and C1-C4 alkyl,
provided that the compound is not PapA.

2. The vector of claim 1, wherein R¹ is —CO₂H or a structure:

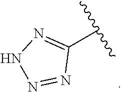 [SMILES: *c1nn[nH]n1]

3. The vector of claim 1, wherein R¹ is —CO₂H.

4. The vector of claim 1, wherein the compound has a structure represented by a formula:

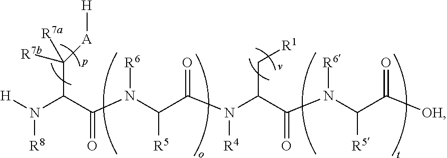

5. The vector of claim 4, wherein the compound has a structure represented by a formula:

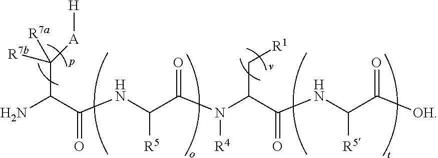

6. The vector of claim 4, wherein the compound has a structure represented by a formula:

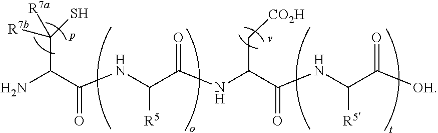

7. The vector of claim 1, wherein the compound has a structure represented by a formula:

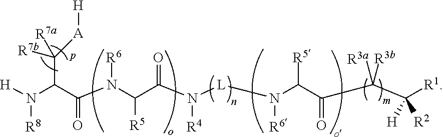

8. The vector of claim 7, wherein the compound has a structure represented by a formula:

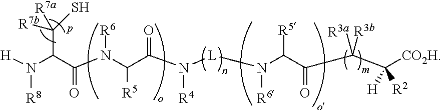

9. The vector of claim 1, further comprising a nucleic acid sequence capable of encoding a phage coat protein.

10. The vector of claim 1, wherein the vector comprises a nucleic acid sequence capable of encoding a leader sequence and one or more of the compounds.

11. A method of making a phage display library, the method comprising:
a) introducing the vector of claim 1 and a vector comprising a phage genome into a host cell; and
b) culturing the host cells under conditions suitable to form phage particles.

12. A method of screening a phage display library, the method comprising:
a) introducing the vector of claim 1 and a vector comprising a phage genome into a host cell;
b) culturing the host cells under conditions suitable to form phage particles;
c) contacting the phage particles with PapB under conditions to allow the compound to form a crosslink, thereby forming a phage particle comprising a cross-linked compound;
d) contacting the phage particles comprising the cross-linked compound with a target molecule to allow binding of the phage particle to the target molecule; and
e) separating phage particles that bind to the target molecule from those that do not bind.

13. A phage comprising a compound, wherein the compound has a structure represented by a formula selected from: 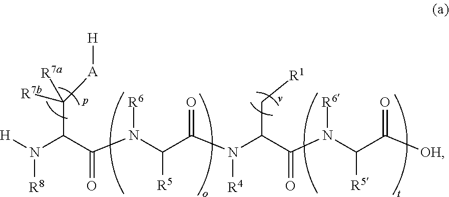
wherein o is 0, 1, 2, 3, 4, 5, 6, 7, 8, or 9;
wherein p is 1 or 2;
wherein t is an integer from 0 to 500;
wherein v is 1, 2, 3, 4, or 5;
wherein A is S or Se;
wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from: 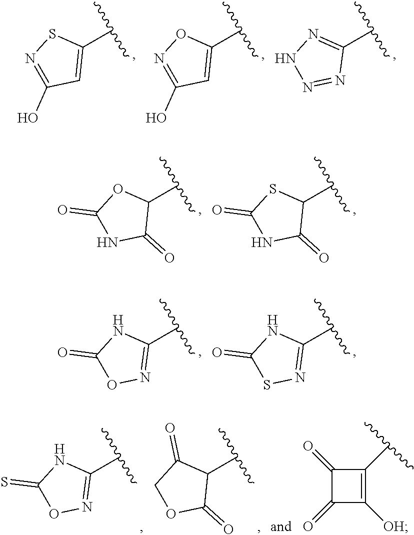
wherein R⁴ is selected from hydrogen and methyl;
wherein each occurrence of R⁵ and R^{5′}, when present, is independently a residue of a side chain of amino acid;
wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle;
wherein each of R^{7a} and R^{7b}, when present, is independently selected from hydrogen and C1-C4 alkyl; and
wherein R⁸ is selected from hydrogen and methyl; and 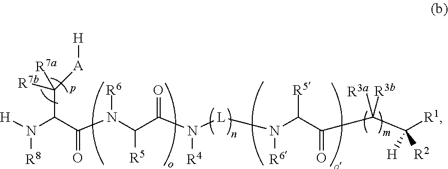
wherein m is 0, 1, 2, 3, or 4;
wherein n is 0 or 1;
wherein each of o and o′ is independently 0, 1, 2, 3, 4, 5, 6, 7, 8, or 9;
wherein p is 1 or 2;
wherein A is S or Se;
wherein L, when present, is selected from C2-C4 alkyl, —(C1-C4 alkyl)(OCH₂CH₂)_{q}, and a structure selected from: 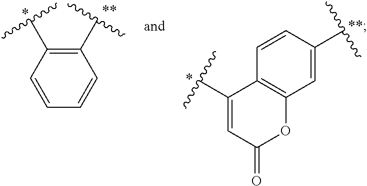
wherein q is 1, 2, 3, or 4;
wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from: 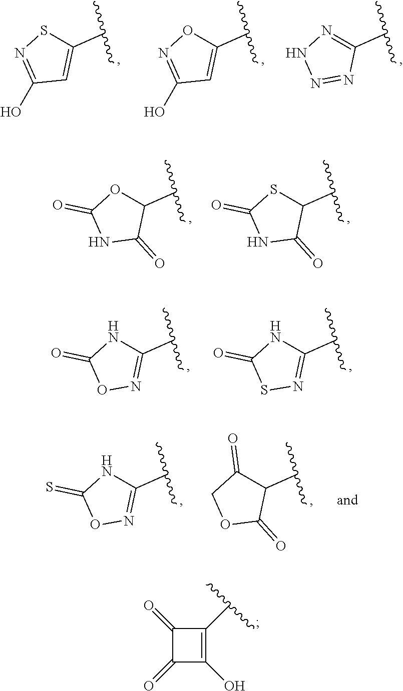
wherein R² is a residue of a side chain of amino acid, provided that the amino acid is not isoleucine or threonine;
wherein each of R^{3a} and R^{3b}, when present, is independently selected from C2-C5 alkynyl, C1-C5 azido, and a residue of a side chain of an amino acid;
wherein R⁴ is selected from hydrogen and methyl;
wherein each occurrence of R⁵ and R^{5′}, when present, is independently a residue of a side chain of amino acid;
wherein each occurrence of R⁶ and R^{6′}, when present, is independently selected from hydrogen and methyl, or wherein R⁶ or R^{6′} is covalently bonded to R⁵ or R^{5′} respectively, and, together with the intermediate atoms, comprise an unsubstituted 5-membered heterocycle;
wherein each of R^{7a} and R⁷, when present, is independently selected from hydrogen and C1-C4 alkyl,
provided that the compound is not PapA.

14. A phage comprising a thioether or selenoether compound, wherein the thioether or selenoether compound has a structure represented by a formula selected from: 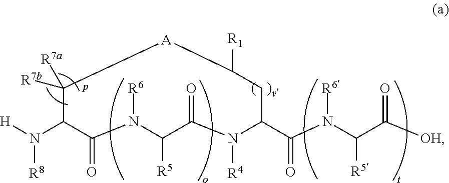
wherein o is 0, 1, 2, 3, 4, 5, 6, 7, 8, or 9;
wherein p is 1 or 2;
wherein t is an integer from 0 to 500;
wherein v′ is 0, 1, 2, 3, or 4;
wherein A is S or Se;
wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from: 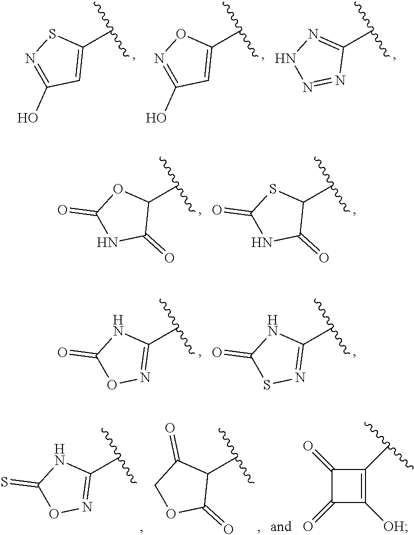
wherein R⁴ is selected from hydrogen and methyl;
wherein each occurrence of R⁵ and R^{5′}, when present, is independently a residue of a side chain of amino acid;
wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle;
wherein each of R^{7a} and R^{7b}, when present, is independently selected from hydrogen and C1-C4 alkyl; and
wherein R⁸ is selected from hydrogen and methyl; and 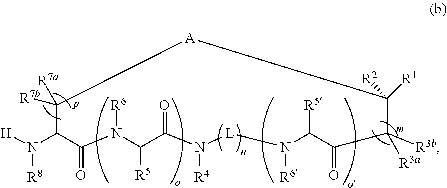
wherein m is 0, 1, 2, 3, or 4;
wherein n is 0 or 1;
wherein each of o and o′ is independently 0, 1, 2, 3, 4, 5, 6, 7, 8, or 9;
wherein p is 1 or 2;
wherein A is S or Se;
wherein L, when present, is selected from C2-C4 alkyl, —(C1-C4 alkyl)(OCH₂CH₂)_{q}, and a structure selected from: 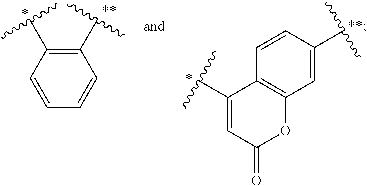
wherein q is 1, 2, 3, or 4;
wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from: 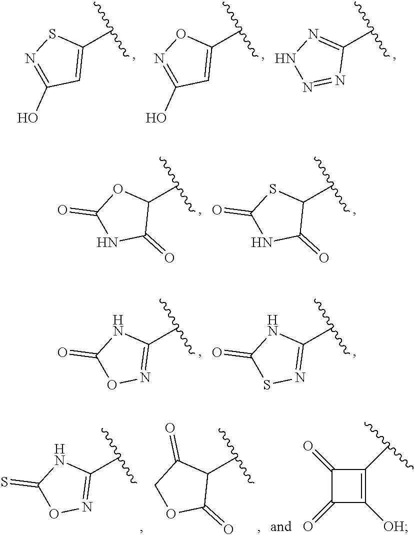
wherein R² is a residue of a side chain of amino acid, provided that the amino acid is not isoleucine or threonine;
wherein each of R^{3a} and R^{3b}, when present, is independently selected from C2-C5 alkynyl, C1-C5 azido, and a residue of a side chain of an amino acid;
wherein R⁴ is selected from hydrogen and methyl;
wherein each occurrence of R⁵ and R^{5′}, when present, is independently a residue of a side chain of amino acid;
wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle;
wherein each of R^{7a} and R^{7b}, when present, is independently selected from hydrogen and C1-C4 alkyl,
provided that the compound is not PapA.

15. A phage display library comprising two or more virus particles, each of the two or more virus particles comprising a compound, wherein each compound has a structure represented by a formula selected from: 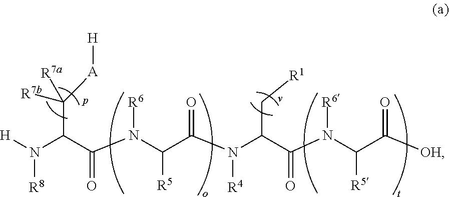
wherein o is 0, 1, 2, 3, 4, 5, 6, 7, 8, or 9;
wherein p is 1 or 2;
wherein t is an integer from 0 to 500;
wherein v is 1, 2, 3, 4, or 5;
wherein A is S or Se;
wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from: 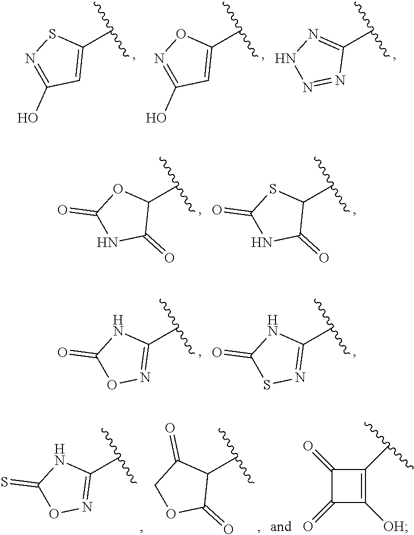
wherein R⁴ is selected from hydrogen and methyl;
wherein each occurrence of R⁵ and R^{5′}, when present, is independently a residue of a side chain of amino acid;
wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle;
wherein each of R^{7a} and R⁷, when present, is independently selected from hydrogen and C1-C4 alkyl; and
wherein R⁸ is selected from hydrogen and methyl; and 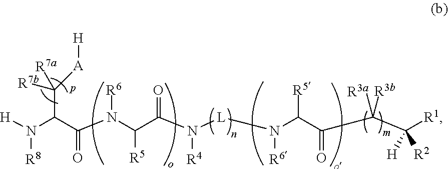
wherein m is 0, 1, 2, 3, or 4;
wherein n is 0 or 1;
wherein each of o and o′ is independently 0, 1, 2, 3, 4, 5, 6, 7, 8, or 9;
wherein p is 1 or 2;
wherein A is S or Se;
wherein L, when present, is selected from C2-C4 alkyl, —(C1-C4 alkyl)(OCH₂CH₂)_{q}, and a structure selected from: 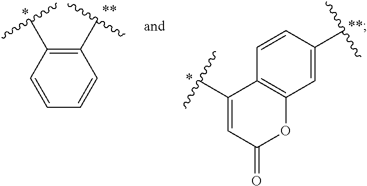
wherein q is 1, 2, 3, or 4;
wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from: 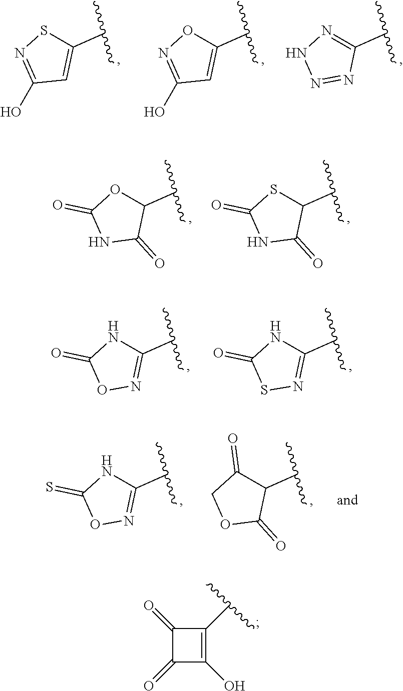
wherein R² is a residue of a side chain of amino acid, provided that the amino acid is not isoleucine or threonine;
wherein each of R^{3a} and R^{3b}, when present, is independently selected from C2-C5 alkynyl, C1-C5 azido, and a residue of a side chain of an amino acid;
wherein R⁴ is selected from hydrogen and methyl;
wherein each occurrence of R⁵ and R^{5′}, when present, is independently a residue of a side chain of amino acid;
wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle;
wherein each of R^{7a} and R^{7b}, when present, is independently selected from hydrogen and C1-C4 alkyl,
provided that the compound is not PapA.

16. A phage display library comprising two or more virus particles, each of the two or more virus particles comprising a thioether or selenoether compound, wherein each thioether or selenoether compound has a structure represented by a formula selected from: 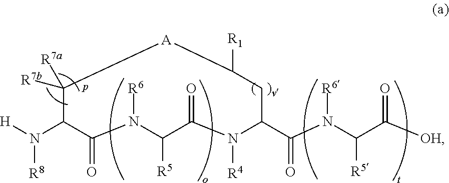
wherein o is 0, 1, 2, 3, 4, 5, 6, 7, 8, or 9;
wherein p is 1 or 2;
wherein t is an integer from 0 to 500;
wherein v′ is 0, 1, 2, or 3;
wherein A is S or Se;
wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from: 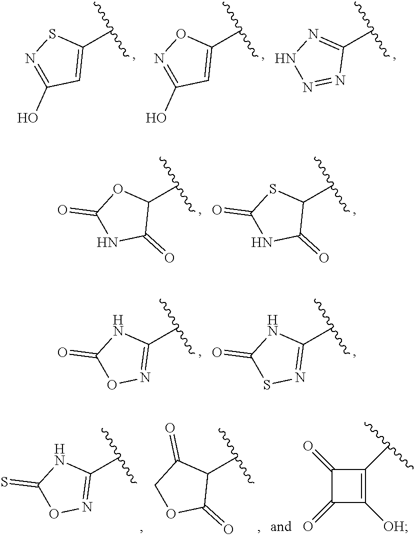
wherein R⁴ is selected from hydrogen and methyl;
wherein each occurrence of R⁵ and R^{5′}, when present, is independently a residue of a side chain of amino acid;
wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle;
wherein each of R^{7a} and R^{7b}, when present, is independently selected from hydrogen and C1-C4 alkyl; and
wherein R¹ is selected from hydrogen and methyl; and 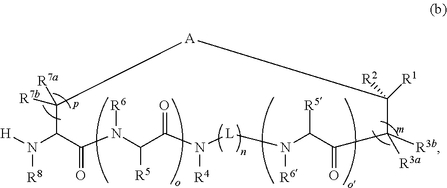
wherein m is 0, 1, 2, 3, or 4;
wherein n is 0 or 1;
wherein each of o and o′ is independently 0, 1, 2, 3, 4, 5, 6, 7, 8, or 9;
wherein p is 1 or 2;
wherein A is S or Se;
wherein L, when present, is selected from C2-C4 alkyl, —(C1-C4 alkyl)(OCH₂CH₂)_{q}, and a structure selected from: 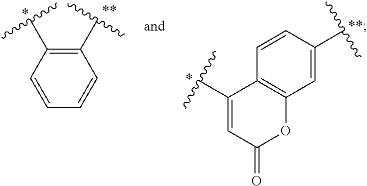
wherein q is 1, 2, 3, or 4;
wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from: 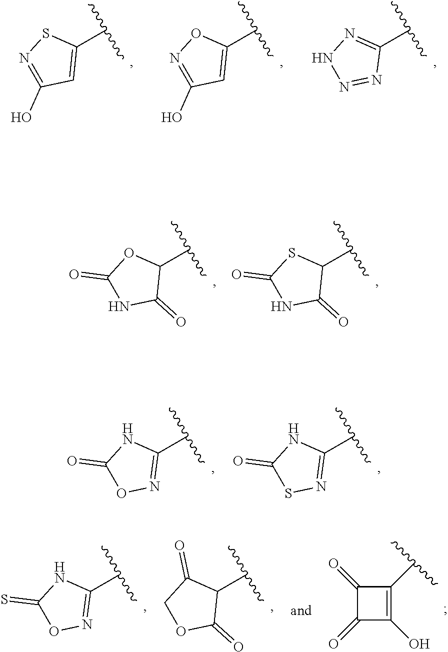
wherein R² is a residue of a side chain of amino acid, provided that the amino acid is not isoleucine or threonine;
wherein each of R^{3a} and R^{3b}, when present, is independently selected from C2-C5 alkynyl, C1-C5 azido, and a residue of a side chain of an amino acid;
wherein R⁴ is selected from hydrogen and methyl;
wherein each occurrence of R⁵ and R^{5′}, when present, is independently a residue of a side chain of amino acid;
wherein each occurrence of R⁶ and R^{6′}, when present, is independently selected from hydrogen and methyl, or wherein R⁶ or R^{6′} is covalently bonded to R⁵ or R^{5′} respectively, and, together with the intermediate atoms, comprise an unsubstituted 5-membered heterocycle;
wherein each of R^{7a} and R⁷, when present, is independently selected from hydrogen and C1-C4 alkyl,
provided that the thioether compound is not PapA.

17. A ribosome display library comprising a plurality of complexes, wherein each complex comprises:
a) a mRNA molecule, wherein the mRNA molecule comprises a nucleotide sequence capable of encoding one or more of the compounds disclosed herein and lacking an in-frame stop codon; and
b) one or more of the compounds or thioether compounds disclosed herein.

18. A method of screening using a ribosome display library, the method comprising:
a) obtaining a plurality of mRNA molecules, each mRNA molecule comprising a nucleotide sequence capable of encoding one or more of the compounds disclosed herein and lacking an in-frame stop codon;
b) incubating the plurality of mRNA molecules under conditions for ribosome translation of the mRNA molecules to produce encoded compounds, whereby complexes each comprising the mRNA molecule that encodes a compound and the encoded compound are formed;
c) contacting the complexes with PapB under conditions to allow the compound to form a crosslink, thereby forming a complex comprising a cross-linked compound;
d) contacting the complexes with a ligand that binds the compound;
e) selecting one or more complexes each displaying a compound able to bind the ligand;
f) isolating the mRNA molecule from the complex bound to the ligand; and
g) reverse transcribing the mRNA molecule to cDNA.

19. A cell-surface display library comprising a host cell, wherein the host cell comprises one or more of the compounds or thioether compounds disclosed herein on the cell surface.

20. A method for making a yeast cell surface display, the method comprising transforming yeast cells with a vector comprising a nucleic acid sequence capable of encoding one or more of the compounds disclosed herein fused to a yeast cell wall protein.
